(19) United States
(12) Patent Application Publication
(10) Pub. No.: US 2025/0263398 A1
(43) Pub. Date: Aug. 21, 2025

(54) PROCESS

(71) Applicant: Assembly Biosciences, Inc., South San Francisco, CA (US)

(72) Inventors: Mark JOHNSON, South San Francisco, CA (US); Michael WALLACE, South San Francisco, CA (US); Roman LAGOUTTE, Basel (CH); Rodolphe BEAUD, Basel (CH)

(73) Assignee: Assembly Biosciences, Inc., South San Francisco, CA (US)

(21) Appl. No.: 18/702,974

(22) PCT Filed: Oct. 19, 2022
(86) PCT No.: PCT/IB2022/060056

§ 371 Date: Apr. 19, 2024

Related U.S. Application Data
(60) Provisional application No. 63/257,697, filed on Oct. 20, 2021.

(51) Int. Cl. C07D 403/08; C07C 49/297; C07F 5/02; C07F 9/09; C07F 9/6503

(52) U.S. Cl. CPC C07D 403/08; C07C 49/297; C07F 5/025; C07F 9/09; C07F 9/65031

(57) ABSTRACT

The present disclosure generally relates to methods of synthesizing compounds useful as modulators of hepatitis B virus core protein assembly as well as novel synthetic intermediates. The methods disclosed may be used in the manufacture of compounds which may have allosteric effector properties against hepatitis B virus (HBV) core protein (Cp), a protein found as a dimer, a multimer, and as the protein shell of the HBV core. As one example, provided herein is a process for preparing compounds which may be useful for treating viral infections, such as hepatitis B.

### FIELD OF THE INVENTION

The present disclosure generally relates to methods of synthesizing compounds useful as modulators of hepatitis B virus core protein assembly as well as novel synthetic intermediates. The methods disclosed may be used in the manufacture of compounds which may have allosteric effector properties against hepatitis B virus (HBV) core protein (Cp), a protein found as a dimer, a multimer, and as the protein shell of the HBV core. As one example, provided herein is a process for preparing compounds which may be useful for treating viral infections, such as hepatitis B.

### BACKGROUND

Hepatitis B (HBV) causes viral hepatitis that can further lead to chronic liver disease and increase the risk of liver cirrhosis and liver cancer (hepatocellular carcinoma). Worldwide, about 2 billion people have been infected with HBV, around 360 million people are chronically infected, and every year HBV infection causes more than one half million deaths. HBV can be spread by body fluids: from mother to child, by sex, and via blood products. Children born to HBV-positive mothers may also be infected, unless vaccinated at birth.

The hepatitis virus particle is composed of a lipid envelope studded with surface protein (HBsAg) that surrounds the viral core. The core is composed of a protein shell, or capsid, built of 120 core protein (Cp) dimers, which in turn contains the relaxed circular DNA (rcDNA) viral genome as well as viral and host proteins. In an infected cell, the genome is found as a covalently closed circular DNA (cccDNA) in the host cell nucleus. The cccDNA is the template for viral RNAs and thus viral proteins. In the cytoplasm, Cp assembles around a complex of full-length viral RNA (the so-called pregenomic RNA or pgRNA and viral polymerase (P). After assembly, P reverse transcribes the pgRNA to rcDNA within the confines of the capsid to generate the DNA-filled viral core.

At present, chronic HBV is primarily treated with nucleos(t)ide analogs (e.g., entecavir) that suppress the virus while the patient remains on treatment, but do not eliminate the infection, even after many years of treatment. Once a patient starts taking nucleos(t)ide analogs, most must continue taking them or risk the possibility of a life-threatening immune response due to viral rebound. Further, nucleotide therapy may lead to the emergence of antiviral drug resistance.

The only FDA approved alternative to nucleos(t)ide analogs is treatment with interferon α or pegylated interferon α. Unfortunately, the adverse event incidence and profile of interferon α can result in poor tolerability, and many patients are unable to complete therapy. Moreover, only a small percentage of patients are considered appropriate for interferon therapy, as only a small subset of patients is likely to have a sustained clinical response to a course of interferon therapy. As a result, interferon-based therapies are used in only a small percentage of all diagnosed patients who elect treatment.

Thus, current HBV treatments can range from palliative to watchful waiting. Nucleotide analogs suppress virus production, treating the symptom, but leave the infection intact. Interferon α has severe side effects and less tolerability among patients and is successful as a finite treatment strategy in only a small minority of patients. There is a clear on-going need for more effective treatments for HBV infections.

The present disclosure relates to alternative and novel synthetic methods for the compounds disclosed in WO 2021/216656 (PCT/US2021/028323), which is hereby incorporated by reference in its entirety.

### SUMMARY OF THE INVENTION

One embodiment of the present disclosure is a method for synthesizing Compound I, or a salt thereof:

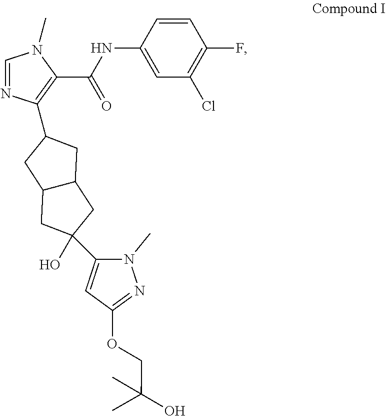 [SMILES: Cn1cnc(C2CC3CC(O)(c4cc(OCC(C)(C)O)nn4C)CC3C2)c1C(=O)Nc1ccc(F)c(Cl)c1]

comprising the steps of:
Forming a vinyl triflate (Compound 2)

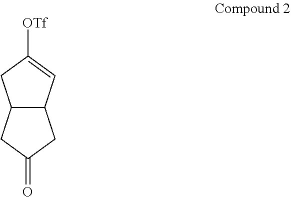 [SMILES: O=C1CC2C=C(OS(=O)(=O)C(F)(F)F)CC2C1]

Forming a vinyl-boronate (Compound 3)

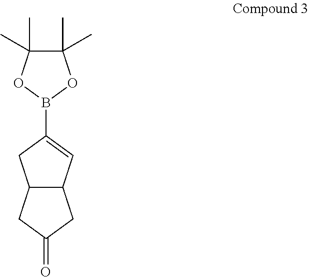

Alkynylating Compound 3 to form Compound 3.1

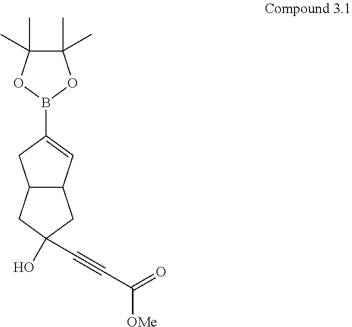 [SMILES: COC(=O)C#CC1(O)CC2C=C(B3OC(C)(C)C(C)(C)O3)CC2C1]

Forming an hydroxypyrazole (Compound 3.2)

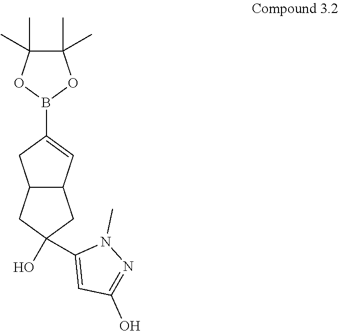 [SMILES: Cn1nc(O)cc1C1(O)CC2C=C(B3OC(C)(C)C(C)(C)O3)CC2C1]

Alkylating Compound 3.2 to form Compound 3.3

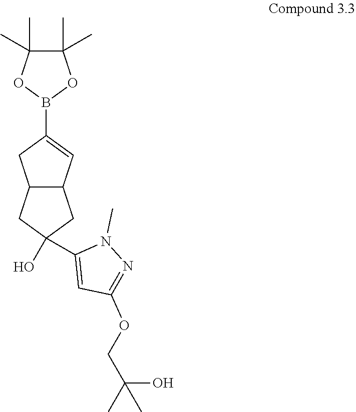 [SMILES: Cn1nc(OCC(C)(C)O)cc1C1(O)CC2C=C(B3OC(C)(C)C(C)(C)O3)CC2C1]

Cross-coupling Compound 3.3 to form a vinyl imidazole (Compound 3.4)

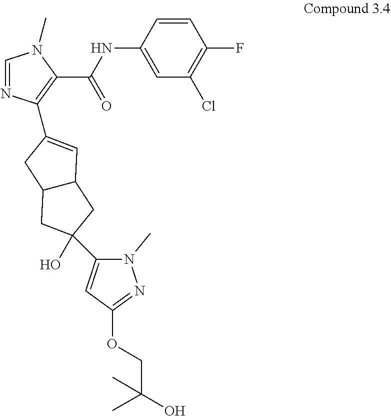 [SMILES: Cn1cnc(C2=CC3CC(O)(c4cc(OCC(C)(C)O)nn4C)CC3C2)c1C(=O)Nc1ccc(F)c(Cl)c1]

Hydrogenating Compound 3.4 to form Compound I

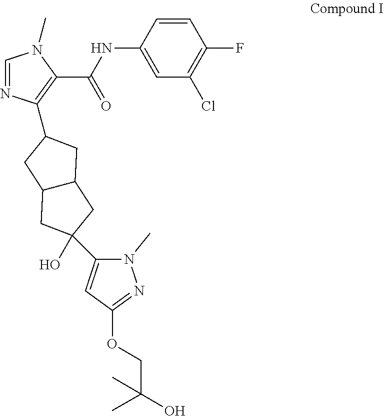 [SMILES: Cn1cnc(C2CC3CC(O)(c4cc(OCC(C)(C)O)nn4C)CC3C2)c1C(=O)Nc1ccc(F)c(Cl)c1]

or a salt thereof.

One embodiment of the present disclosure is a method for synthesizing Compound I:

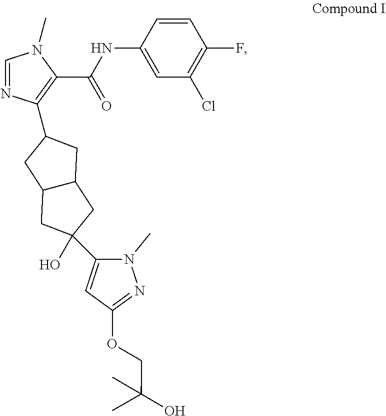 [SMILES: Cn1cnc(C2CC3CC(O)(c4cc(OCC(C)(C)O)nn4C)CC3C2)c1C(=O)Nc1ccc(F)c(Cl)c1]

or a salt thereof; comprising the step of converting (such as hydrogenating) Compound 3.4 to Compound I or a salt thereof,

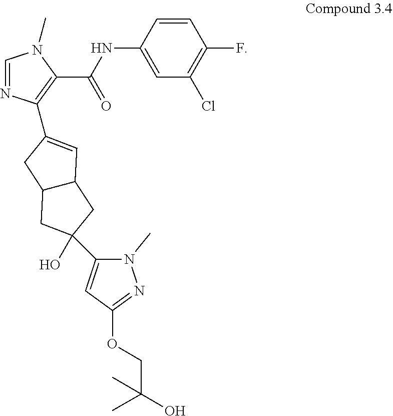 [SMILES: Cn1cnc(C2=CC3CC(O)(c4cc(OCC(C)(C)O)nn4C)CC3C2)c1C(=O)Nc1ccc(F)c(Cl)c1]

One embodiment of the present disclosure is a method for synthesizing Compound I, or a salt thereof, comprising the steps of:
i. converting Compound 3.3 to Compound 3.4; and
ii. converting Compound 3.4 to Compound I, or a salt thereof,

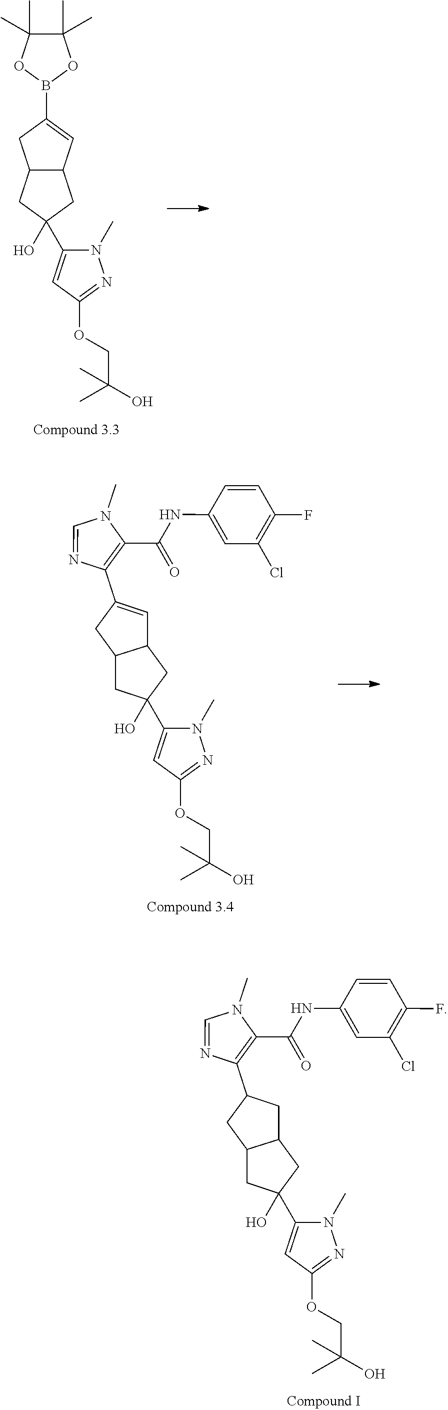 [SMILES: Cn1nc(OCC(C)(C)O)cc1C1(O)CC2C=C(B3OC(C)(C)C(C)(C)O3)CC2C1 ; Cn1cnc(C2=CC3CC(O)(c4cc(OCC(C)(C)O)nn4C)CC3C2)c1C(=O)Nc1ccc(F)c(Cl)c1 ; Cn1cnc(C2CC3CC(O)(c4cc(OCC(C)(C)O)nn4C)CC3C2)c1C(=O)Nc1ccc(F)c(Cl)c1]

One embodiment of the present disclosure is a method for synthesizing Compound I, or a salt thereof, comprising the steps of:
i. converting Compound 3.2 to Compound 3.3;
ii. converting Compound 3.3 to Compound 3.4; and
iii. converting Compound 3.4 to Compound I, or a salt thereof,

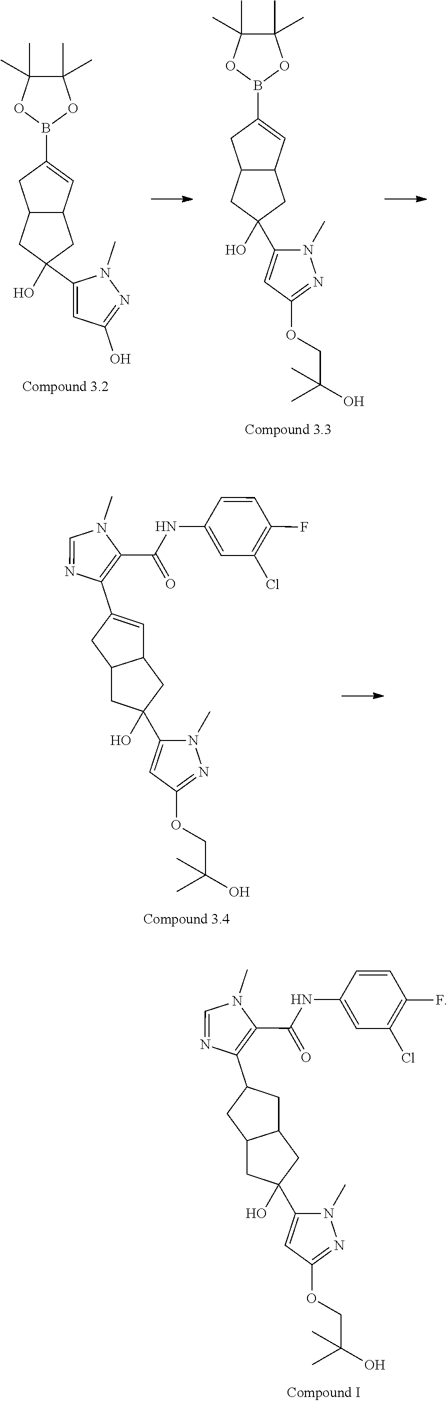 [SMILES: Cn1nc(O)cc1C1(O)CC2C=C(B3OC(C)(C)C(C)(C)O3)CC2C1 ; Cn1nc(OCC(C)(C)O)cc1C1(O)CC2C=C(B3OC(C)(C)C(C)(C)O3)CC2C1 ; Cn1cnc(C2=CC3CC(O)(c4cc(OCC(C)(C)O)nn4C)CC3C2)c1C(=O)Nc1ccc(F)c(Cl)c1 ; Cn1cnc(C2CC3CC(O)(c4cc(OCC(C)(C)O)nn4C)CC3C2)c1C(=O)Nc1ccc(F)c(Cl)c1]

One embodiment of the present disclosure is a method for synthesizing Compound I, or a salt thereof, comprising the steps of:
i. converting Compound 3.1 to Compound 3.2;
ii. converting Compound 3.2 to Compound 3.3;
iii. converting Compound 3.3 to Compound 3.4; and
iv. converting Compound 3.4 to Compound I, or a salt thereof,

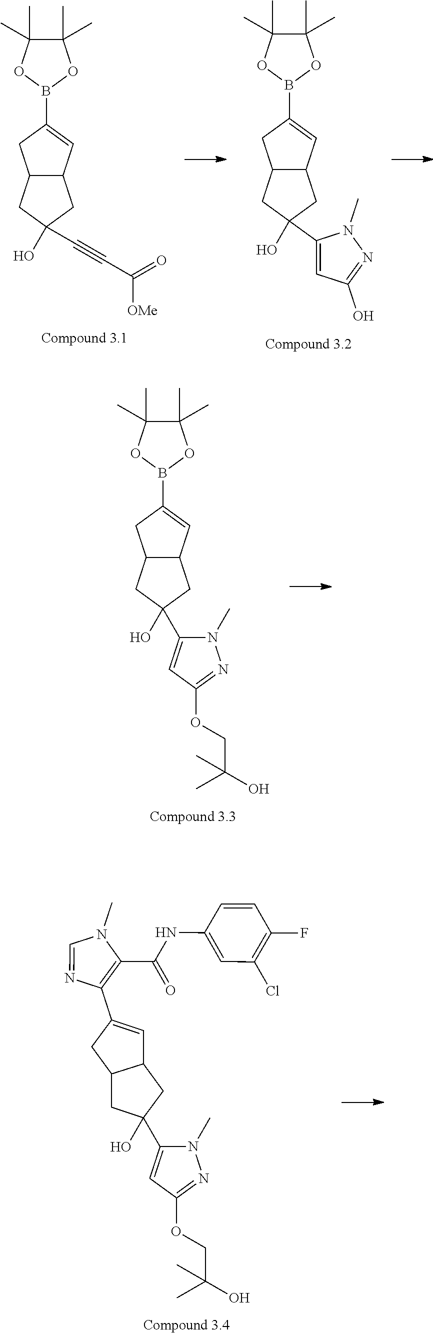 [SMILES: COC(=O)C#CC1(O)CC2C=C(B3OC(C)(C)C(C)(C)O3)CC2C1 ; Cn1nc(O)cc1C1(O)CC2C=C(B3OC(C)(C)C(C)(C)O3)CC2C1 ; Cn1nc(OCC(C)(C)O)cc1C1(O)CC2C=C(B3OC(C)(C)C(C)(C)O3)CC2C1 ; Cn1cnc(C2=CC3CC(O)(c4cc(OCC(C)(C)O)nn4C)CC3C2)c1C(=O)Nc1ccc(F)c(Cl)c1]

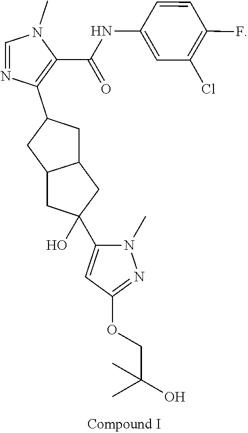 [SMILES: Cn1cnc(C2CC3CC(O)(c4cc(OCC(C)(C)O)nn4C)CC3C2)c1C(=O)Nc1ccc(F)c(Cl)c1]

One embodiment of the present disclosure is a method for synthesizing Compound I, or a salt thereof, comprising the steps of:
i. converting Compound 3 to Compound 3.1;
ii. converting Compound 3.1 to Compound 3.2;
iii. converting Compound 3.2 to Compound 3.3;
iv. converting Compound 3.3 to Compound 3.4; and
v. converting Compound 3.4 to Compound I, or a salt thereof,

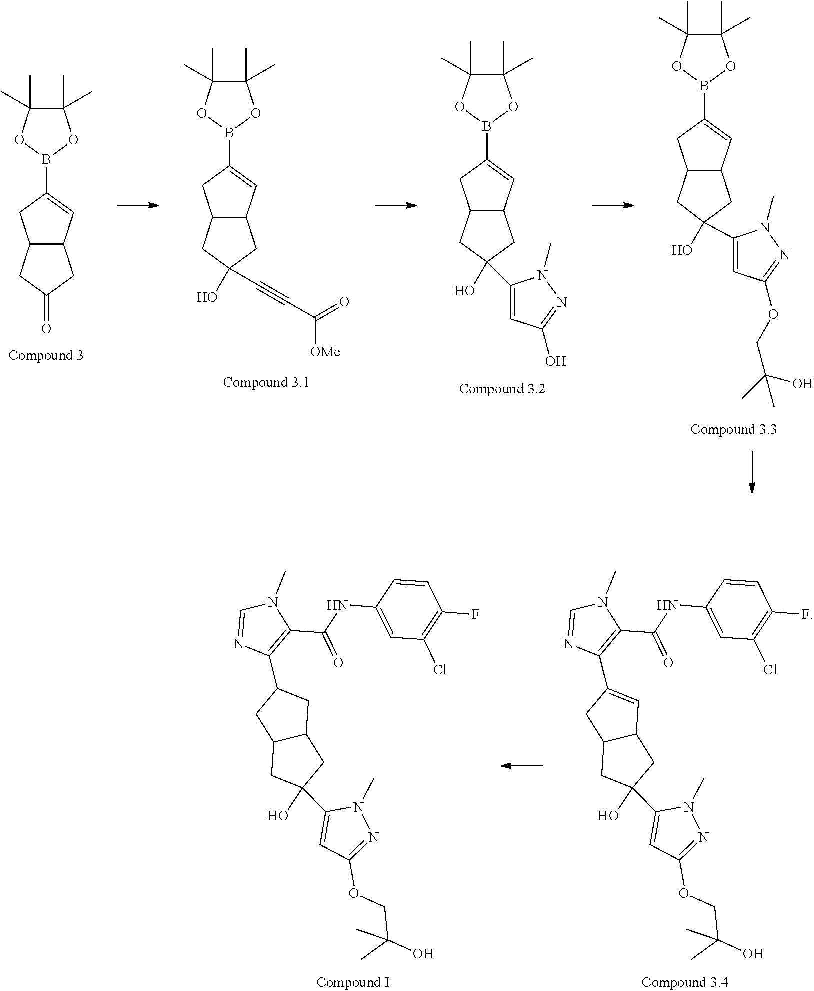

In one aspect, the Compound I is a mixture of diastereomers. In one aspect, the Compound I is stereochemically pure.

In one aspect, Compound I is the diastereomer Compound I(a):

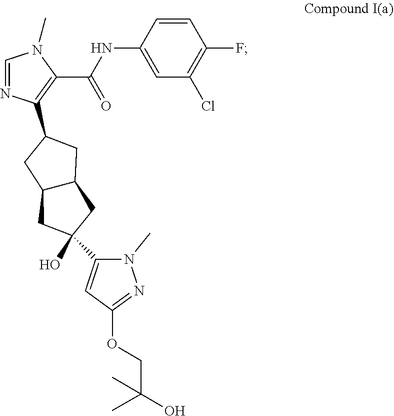 [SMILES: Cn1cnc([C@@H]2C[C@@H]3C[C@@](O)(c4cc(OCC(C)(C)O)nn4C)C[C@@H]3C2)c1C(=O)Nc1ccc(F)c(Cl)c1]

or a salt thereof.

In one aspect, there is provided the intermediate Compound 3.1:

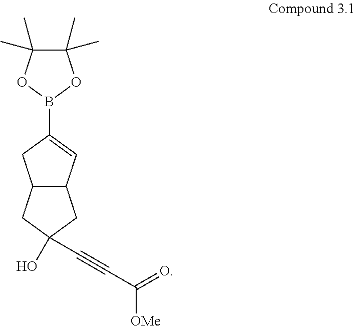 [SMILES: COC(=O)C#CC1(O)CC2C=C(B3OC(C)(C)C(C)(C)O3)CC2C1]

In one embodiment, there is provided the intermediate Compound 3.1a:

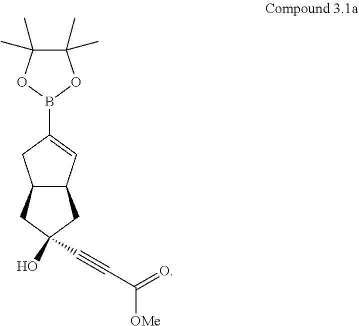 [SMILES: COC(=O)C#C[C@]1(O)C[C@H]2CC(B3OC(C)(C)C(C)(C)O3)=C[C@H]2C1]

In one aspect, there is provided the intermediate Compound 3.2:

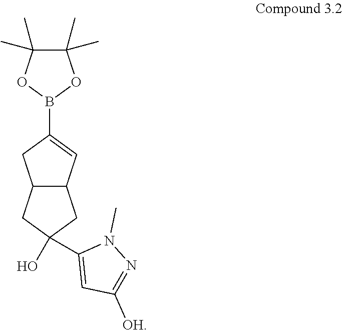 [SMILES: Cn1nc(O)cc1C1(O)CC2C=C(B3OC(C)(C)C(C)(C)O3)CC2C1]

In one embodiment, there is provided the intermediate Compound 3.2a:

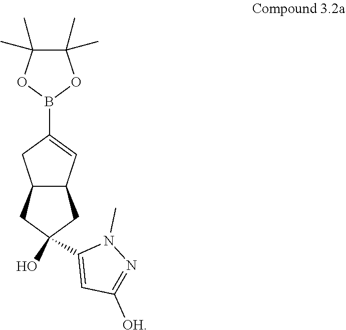 [SMILES: Cn1nc(O)cc1[C@]1(O)C[C@H]2CC(B3OC(C)(C)C(C)(C)O3)=C[C@H]2C1]

In one aspect, there is provided the intermediate Compound 3.3:

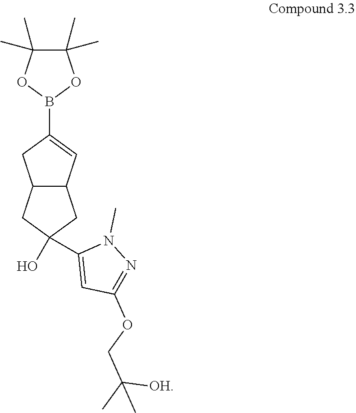 [SMILES: Cn1nc(OCC(C)(C)O)cc1C1(O)CC2C=C(B3OC(C)(C)C(C)(C)O3)CC2C1]

In one embodiment, there is provided the intermediate Compound 3.3a:

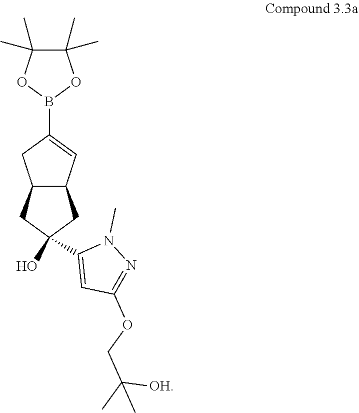 [SMILES: Cn1nc(OCC(C)(C)O)cc1[C@]1(O)C[C@H]2CC(B3OC(C)(C)C(C)(C)O3)=C[C@H]2C1]

In one aspect, there is provided the intermediate Compound 3.4:

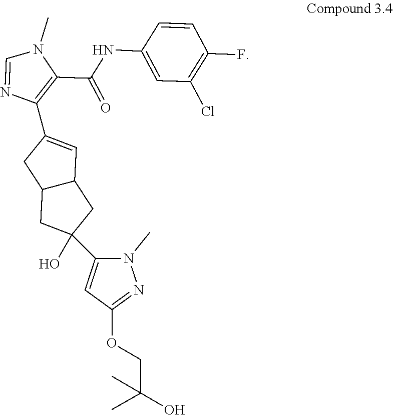 [SMILES: Cn1cnc(C2=CC3CC(O)(c4cc(OCC(C)(C)O)nn4C)CC3C2)c1C(=O)Nc1ccc(F)c(Cl)c1]

In one embodiment, there is provided the intermediate Compound 3.4a:

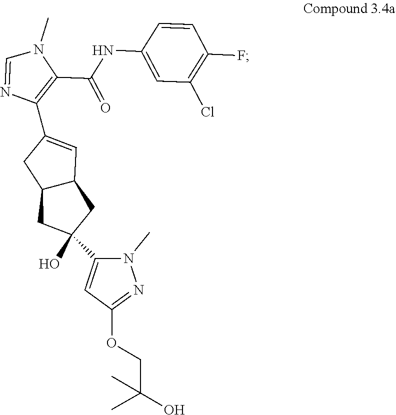 [SMILES: Cn1cnc(C2=C[C@H]3C[C@](O)(c4cc(OCC(C)(C)O)nn4C)C[C@H]3C2)c1C(=O)Nc1ccc(F)c(Cl)c1]

### DETAILED DESCRIPTION OF THE DISCLOSURE

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

Unless otherwise stated, the following terms used in the specification and claims have the following meanings set out below.

As used herein, “Compound I” refers to N-(3-Chloro-4-fluorophenyl)-4-(5-hydroxy-5-(3-(2-hydroxy-2-methylpropoxy)-1-methyl-1H-pyrazol-5-yl)octahydropentalen-2-yl)-1-methyl-1H-imidazole-5-carboxamide:

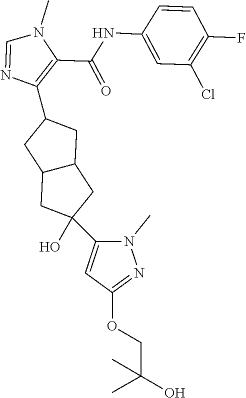 [SMILES: Cn1cnc(C2CC3CC(O)(c4cc(OCC(C)(C)O)nn4C)CC3C2)c1C(=O)Nc1ccc(F)c(Cl)c1]

As used herein, “Compound I(a)” refers to N-(3-chloro-4-fluorophenyl)-4-((2s,3aR,5r,6aS)-5-hydroxy-5(3-(2-hydroxy-2-methylpropoxy)-1-methyl-1H-pyrazol-5-yl)octahydropentalen-2-yl)-1-methyl-1H-imidazole-5-carboxamide:

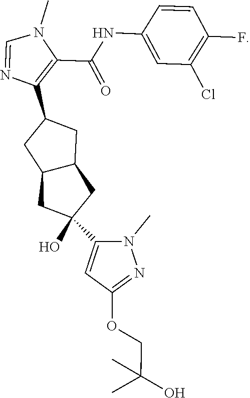 [SMILES: Cn1cnc([C@@H]2C[C@@H]3C[C@@](O)(c4cc(OCC(C)(C)O)nn4C)C[C@@H]3C2)c1C(=O)Nc1ccc(F)c(Cl)c1]

Unless the context requires otherwise, throughout this specification and claims, the words “comprise,” “comprising” and the like are to be construed in an open, inclusive sense; the words “a” “an” and the like are to be considered as meaning at least one and are not limited to just one; and the term “about” is to be construed as meaning plus or minus 10%. Terms not specifically defined herein should be given the meanings that would be given to them by one of skill in the art in light of the disclosure and the context.

In certain cases, depicted substituents may contribute to optical or stereoisomerism. Compounds having the same molecular formula but differing in the nature or sequence of bonding of their atoms or in the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers”. A single diastereoisomer compound may form one aspect of the present disclosure.

When a compound has an asymmetric center, for example when it is bonded to four different groups, a pair of enantiomers is possible. An enantiomer can be characterized by the absolute configuration of its asymmetric center and is designated (R) or (S) according to the rules of Cahn and Prelog (Cahn et al., 1966, Angew. Chem. 78: 413-447, Angew. Chem., Int. Ed. Engl. 5: 385-414 (errata: Angew. Chem., Int. Ed. Engl. 5:511); Prelog and Helmchen, 1982, Angew. Chem. 94: 614-631, Angew. Chem. Internat. Ed. Eng. 21: 567-583; Mata and Lobo, 1993, Tetrahedron: Asymmetry 4: 657-668) or can be characterized by the manner in which the molecule rotates the plane of polarized light and is designated dextrorotatory or levorotatory (namely, as (+)- or (−)-isomers, respectively). A chiral compound can exist as either an individual enantiomer or as a mixture thereof. A mixture containing equal proportions of enantiomers is called a “racemic mixture”.

The compounds of the disclosure may exist as stereoisomers. The term “stereoisomers” when used herein consist of all enantiomers or diastereomers. As noted, these compounds may be designated by the symbols “(+),” “(−),” “R” or “S,” depending on the configuration of substituents around the stereogenic carbon atom. The present disclosure encompasses various stereoisomers of these compounds and mixtures thereof. Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol [symbol] denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic ring may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of anew stereocenter or during the transformation of a pre-existing one, are well known in the art. Stereoselective syntheses encompass both enantiomeric and diastereoselective transformations and may involve the use of chiral auxiliaries. For examples, see Carreira and Kvaerno, Classics in Stereoselective Synthesis, Wiley-VCH: Weinheim, 2009.

The terms “individual,” “patient,” or “subject” are used interchangeably and include any animal, including mammals, preferably mice, rats, other rodents, rabbits, dogs, cats, swine, cattle, sheep, horses, or primates, and most preferably humans. The compounds or pharmaceutical compositions of the disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, dogs, primates, and the like). The mammal treated in the methods of the disclosure is desirably a mammal in which treatment of HBV infection is desired.

The term “modulation” includes antagonism (e.g., inhibition), agonism, partial antagonism and/or partial agonism.

The term “pharmaceutically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, fillers, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers, diluents or excipients.

The term “salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various cations. Examples of such salts include alkali metal or alkaline earth metal salts, particularly calcium, magnesium, sodium, lithium, zinc, potassium, and iron salts. Compounds included in the present compositions that include a basic or acidic moiety may also form salts with various amino acids. The compounds of the disclosure may contain both acidic and basic groups; for example, one amino and one carboxylic acid group. In such a case, the compound can exist as an acid addition salt, a zwitterion, or a base salt.

The term “therapeutically effective amount” or “effective amount” as used herein refers to the amount of the subject compound that will elicit the biological or medical response of a tissue, system or animal, (e.g., mammal or human) that is being sought by the researcher, veterinarian, medical doctor or other clinician. The compounds or pharmaceutical compositions of the disclosure are administered in therapeutically effective amounts to treat a disease. Alternatively, a therapeutically effective amount of a compound is the quantity required to achieve a desired therapeutic and/or prophylactic effect. The “therapeutically effective amount” will vary depending on the compound, the disease and its severity and the age, weight, etc., of the mammal to be treated.

The term “treating” includes any effect, e.g., lessening, reducing, modulating, or eliminating, via disruption of HBV core protein assembly, that results in the improvement of the disease. “Disruption” includes inhibition of HBV viral assembly and infection. The compounds disclosed herein can exist in solvated forms with pharmaceutically acceptable solvents such as water, ethanol, and the like, and it is intended that the disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the disclosure can generally be prepared by following procedures analogous to those disclosed in the examples herein by substituting an isotopically labeled reagent for a non-isotopically labeled reagent.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood or liver). Prodrugs are well known in the art (for example, see Rautio, Kumpulainen, et al., Nature Reviews Drug Discovery 2008, 7, 255).

In certain embodiments of the present disclosure, the compounds disclosed herein are “stereochemically pure.” A stereochemically pure compound has a level of stereochemical purity that would be recognized as “pure” by those of skill in the art. Of course, this level of purity may be less than 100%. In certain embodiments, “stereochemically pure” designates a compound that is substantially free, i.e. at least about 85% or more, of alternate isomers. In particular embodiments, the compound is at least about 85%, about 90%, about 91%, about 92%, about 93%, about 94%, about 95%, about 96%, about 97%, about 98%, about 99%, about 99.5% or about 99.9% free of other isomers.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(+)” in nomenclature, but the skilled artisan will recognize that a structure may denote a chiral center implicitly.

In one aspect, Compound I is a mixture of diastereomers. In one embodiment, Compound I(a) is formed as the predominant stereoisomer according to the routes described herein. The term “stereochemically pure” in relation to Compound I(a) means that Compound I(a) is dominated by one diastereomer, for example there is less than about 20% by weight of other diastereomers present (e.g., Compounds I(b), I(c) and/or I(d)), such as less than about 15%, less than about 10%, less than about 5%, less than about 2%, less than about 1%, or less than about 0.5% by weight.

In an embodiment, Compound I is substantially Compound I(a), such as Compound I comprises Compound I(a) and less than 10% by HPLC area of the other diastereoisomers (Compounds I(b), I(c) and I(d)). In an embodiment, Compound I comprises Compound I(a) and less than 5% (such as less than 3%, less than 2%, or less than 1%) by HPLC area of the other diastereoisomers.

Suitably, Compound I essentially consists of a single diastereomer. Suitably, Compound I consists of a single diastereomer. In an embodiment, the single diastereomer is Compound I(a), or a salt thereof. In an embodiment, Compound I(a), or a salt thereof, is stereochemically pure.

At various places in the present specification, values are disclosed in groups or in ranges. It is specifically intended that the description include all individual sub-combination of the members of such groups and ranges and any combination of the various endpoints of such groups or ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as,” “including,” or “for example,” is intended merely to illustrate better the present teachings and does not pose a limitation on the scope of the invention unless claimed.

### Synthesis

Generally, the compounds of the invention can be prepared, isolated or obtained by any method apparent to those of skill in the art. Exemplary methods of preparation are illustrated by the following schemes and description.

Example 55 of WO 2021/216656 (PCT/US2021/028323), herein incorporated by reference, provides one embodiment to prepare Compound I: N-(3-Chloro-4-fluorophenyl)-4-(5-hydroxy-5-(3-(2-hydroxy-2-methylpropoxy)-1-methyl-1H-pyrazol-5-yl)octahydropentalen-2-yl)-1-methyl-1H-imidazole-5-carboxamide:
Example 55, PCT '323

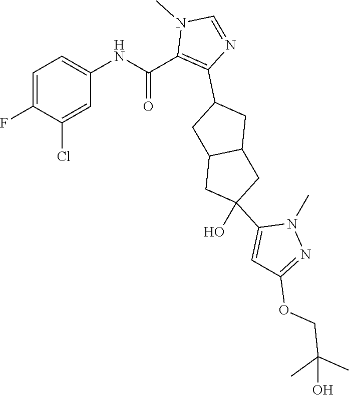 [SMILES: Cn1cnc(C2CC3CC(O)(c4cc(OCC(C)(C)O)nn4C)CC3C2)c1C(=O)Nc1ccc(F)c(Cl)c1]

N-(3-Chloro-4-fluorophenyl)-4-(5-hydroxy-5-(3-(2-hydroxy-2-methylpropoxy)-1-methyl-1H-pyrazol-5-yl)octahydropentalen-2-yl)-1-methyl-1H-imidazole-5-carboxamide.

MeMgBr (3 M in DEE, 0.59 mL, 1.78 mmol) was added slowly to a stirred solution of ethyl 2-((5-(5-(5-((3-chloro-4-fluorophenyl)carbamoyl)-1-methyl-1H-imidazol-4-yl)-2-hydroxy-octahydropentalen-2-yl)-1-methyl-1H-pyrazol-3-yl)oxy)acetate (0.5 g, 0.89 mmol) in dry THF (5 mL) at 0° C. in an inert atmosphere. The reaction mixture was stirred at RT for 2 h. The progress of the reaction was monitored by TLC. After completion, the reaction mixture was quenched with ice cold water and extracted with ethyl acetate. The organic layer was collected; washed with brine; dried over anhydrous sodium sulphate and concentrated under reduced pressure. The crude compound was purified by CombiFlash® column chromatography followed by prep. HPLC to N-(3-chloro-4-fluorophenyl)-4-(5-hydroxy-5-(3-(2-hydroxy-2-methylpropoxy)-1-methyl-1H-pyrazol-5-yl)octahydropentalen-2-yl)-1-methyl-1H-imidazole-5-carboxamide (0.501 g, 61%) as an off white solid. TLC: 5% MeOH in DCM (R_{f}: 0.4); ¹H NMR (400 MHz, DMSO-d₆): δ 10.22 (s, 1H), 7.96 (dd, J=6.8 Hz, 2.4 Hz, 1H), 7.65 (s, 1H), 7.59-7.52 (m, 1H), 7.40 (t, J=9.6 Hz, 1H), 5.52 (s, 1H), 5.23 (s, 1H), 4.53 (s, 1H), 3.75-3.70 (m, 5H), 3.67 (s, 3H), 3.26-3.20 (m, 1H), 2.50-2.44 (m, 2H), 2.20-2.06 (m, 4H), 1.90-1.80 (m, 4H), 1.13 (s, 6H) ppm. MS calcd. for C₂₇H₃₃ClFN₅O₄: 545.2; Found: 546.3 [M+1]⁺.

The product of Example 55 may also be referred to herein as Compound I:

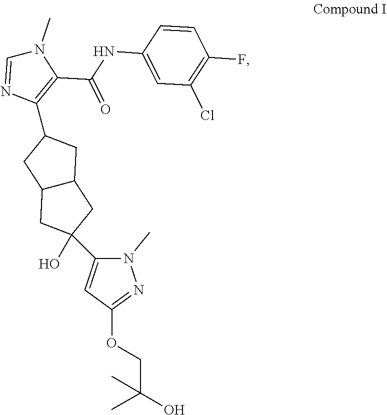 [SMILES: Cn1cnc(C2CC3CC(O)(c4cc(OCC(C)(C)O)nn4C)CC3C2)c1C(=O)Nc1ccc(F)c(Cl)c1]

N-(3-chloro-4-fluorophenyl)-4-(5-hydroxy-5-(3-(2-hydroxy-2-methylpropoxy)-1-methyl-1H-pyrazol-5-yl)octahydropentalen-2-yl)-1-methyl-1H-imidazole-5-carboxamide (Compound I). Following alternative naming conventions may provide different chemical names.

As will be appreciated by those skilled in the art, Compound I is a mixture of diastereomers. Therefore, stereoisomerically dominant or stereoisomerically purified compounds, as encompassed within the phrase “stereochemically pure” as used hereinabove, may form one aspect of the present disclosure. The diastereomers of Compound I include:

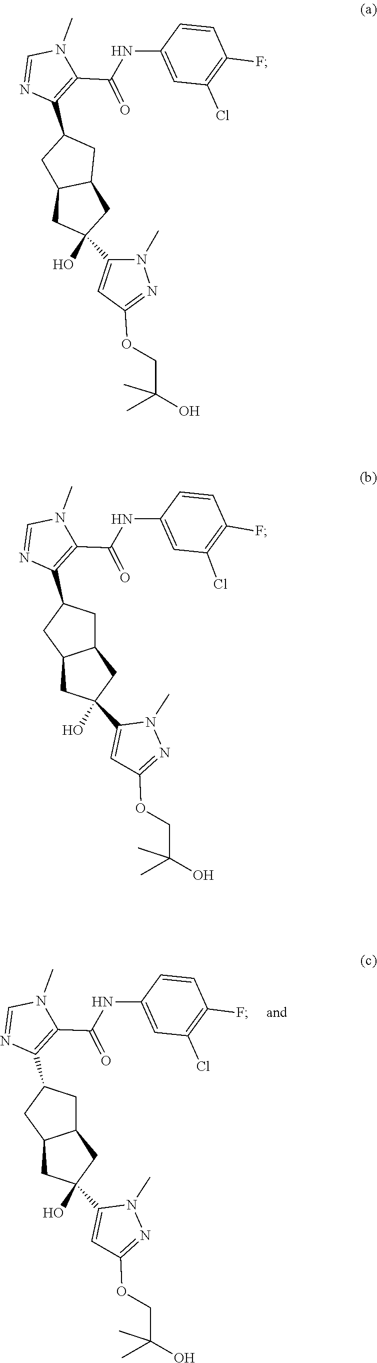 [SMILES: Cn1cnc([C@@H]2C[C@@H]3C[C@@](O)(c4cc(OCC(C)(C)O)nn4C)C[C@@H]3C2)c1C(=O)Nc1ccc(F)c(Cl)c1 ; Cn1cnc([C@@H]2C[C@@H]3C[C@](O)(c4cc(OCC(C)(C)O)nn4C)C[C@@H]3C2)c1C(=O)Nc1ccc(F)c(Cl)c1 ; Cn1cnc([C@H]2C[C@@H]3C[C@@](O)(c4cc(OCC(C)(C)O)nn4C)C[C@@H]3C2)c1C(=O)Nc1ccc(F)c(Cl)c1 ; "and"]

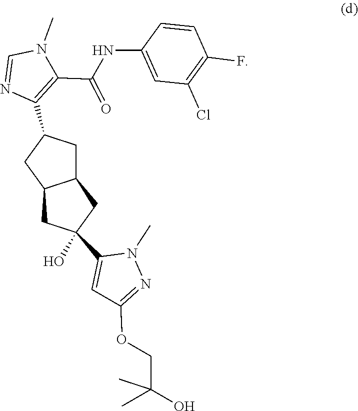 [SMILES: Cn1cnc([C@H]2C[C@@H]3C[C@](O)(c4cc(OCC(C)(C)O)nn4C)C[C@@H]3C2)c1C(=O)Nc1ccc(F)c(Cl)c1]

As noted herein, the present disclosure relates to alternative and novel synthetic methods to prepare Compound I, as well as each of Ia, Ib, Ic, and Id.

Suitably, the diastereomer of Compound I is Compound 1(a).

### Synthesis Methods

### Formation of Compound II

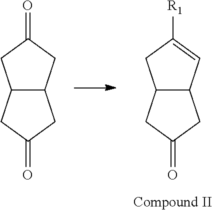

Compound II, where R₁ is a suitable leaving group such as triflate, mesylate or tosylate, may be formed by reacting Compound 1 (tetrahydropentalene-2,5(1H,3H)-dione) with a suitable strong base (such as butyl lithium or lithium hexamethyldisilazane), followed by addition of a suitable leaving group reagent (such as trifluoromethanesulfonic anhydride, methanesulfonic anhydride, or toluenesulfonic anhydride). Suitably, R₁ is triflate (Compound 2).

### Formation of Compound III

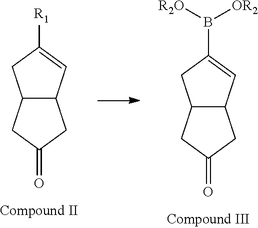

Compound III, wherein each R₂ is independently hydrogen, alkyl or phenyl, or both are Joined to form a cyclic boronic ester (such as pinacol, neopentyl or catechol boronic esters), may be formed by reacting Compound II with a suitable boron reagent (such as bis(pinacolato)diboron) in the presence of a palladium catalyst (such as Pd(dppf)Cl₂) and a suitable base (such as potassium carbonate). Suitably, both R₂ are joined to form a pinacol boronic ester (Compound 3).

### Formation of Compound IV

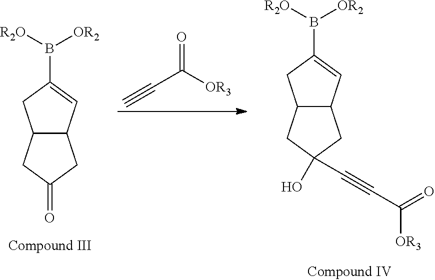

Compound IV, wherein R₃ is C₁₋₄alkyl (such as methyl or ethyl), may be formed by reacting Compound III with an organometallic formed from the reaction of C₁₋₄alkyl propiolate with a suitable strong base (such as butyl lithium or lithium hexamethyldisilazane). Suitably, in Compound IV both R₂ are joined to form a pinacol boronic ester and R₃ is methyl (Compound 3.1).

### Formation of Compound V

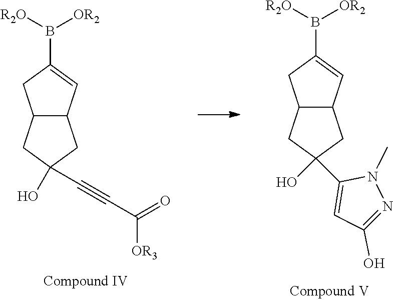

Compound V may be formed by reacting Compound IV with methyl hydrazine or a salt of methyl hydrazine (such as methylhydrazine sulfate) in a suitable solvent (such as toluene) and with heating (such as at >60° C., >70° C., >80° C., or about 90° C.). If a salt of methyl hydrazine is used, then a suitable base (such as triethylamine, or DIPEA) is also required in the reaction to break the salt. Suitably, in Compound V both R₂ are joined to form a pinacol boronic ester (Compound 3.2).

### Formation of Compound VI

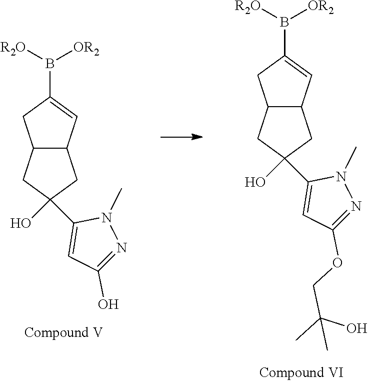

Compound VI may be formed by reacting Compound V with a suitable base (such as potassium carbonate) followed by addition of a suitable alkylating agent (such as isobutylene oxide). Suitably, in Compound VI both R₂ are joined to form a pinacol boronic ester (Compound 3.3).

### Formation of Compound 3.4

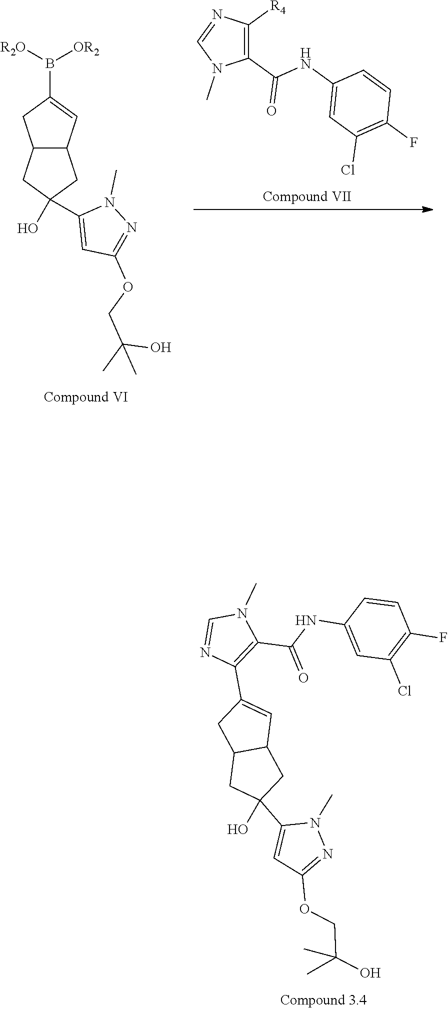

Compound 3.4 may be formed by carrying out a cross-coupling reaction between Compound VI and Compound VII, wherein R₄ is a suitable leaving group (such as bromo, iodo, or triflate). The cross-coupling reaction proceeds in the presence of a suitable base (such as potassium carbonate, cesium carbonate of potassium acetate) and a suitable palladium catalyst (such as Pd(PPh₃)₄, Pd(dppf)Cl₂, or cataCXium® A Pd G3—mesylate[(di(1-adamantyl)-n-butylphosphine)-2-(2′-amino-1,1′-biphenyl)]palladium(II)) with heating (such as at >50° C., >60° C., >70° C., or about 80° C.) in a suitable solvent (such as dioxane, DMA, NMP and/or water).

Compound VII may be synthesized by reaction of 3-chloro-4-fluoroaniline with a suitable imidazole acid using standard amide coupling methodology (e.g. HATU, EDCI, T3P coupling reagents or via acid chloride).

### Formation of Compound I

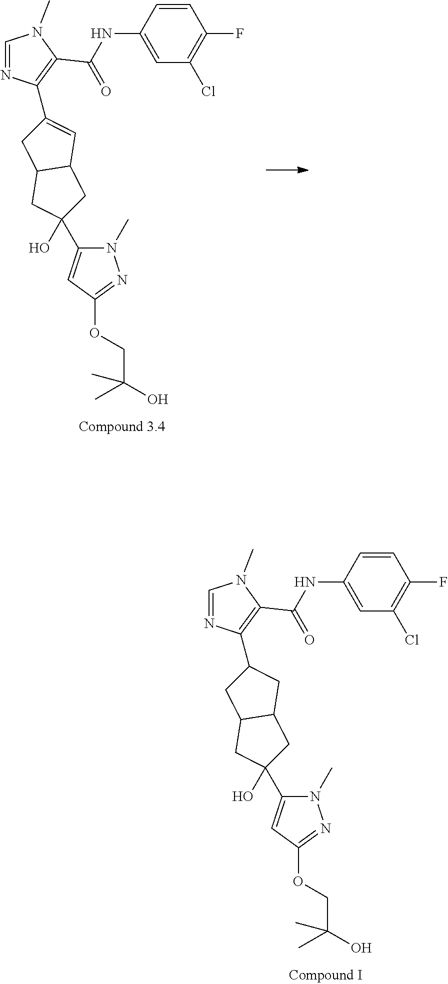 [SMILES: Cn1cnc(C2=CC3CC(O)(c4cc(OCC(C)(C)O)nn4C)CC3C2)c1C(=O)Nc1ccc(F)c(Cl)c1 ; Cn1cnc(C2CC3CC(O)(c4cc(OCC(C)(C)O)nn4C)CC3C2)c1C(=O)Nc1ccc(F)c(Cl)c1]

Compound I may be formed by hydrogenating Compound 3.4 over a suitable supported metal catalyst (such as palladium on carbon, or platinum on carbon) in a suitable solvent (such as ethanol, acetonitrile, ethyl acetate, acetone or THF). The reaction is typically carried out at −5 to 30° C., such as at about −5 to 0° C.

In an embodiment there is provided a method of synthesizing Compound I, or a salt thereof, comprising the step of converting (such as hydrogenating) Compound 3.4 to Compound I or a salt thereof:

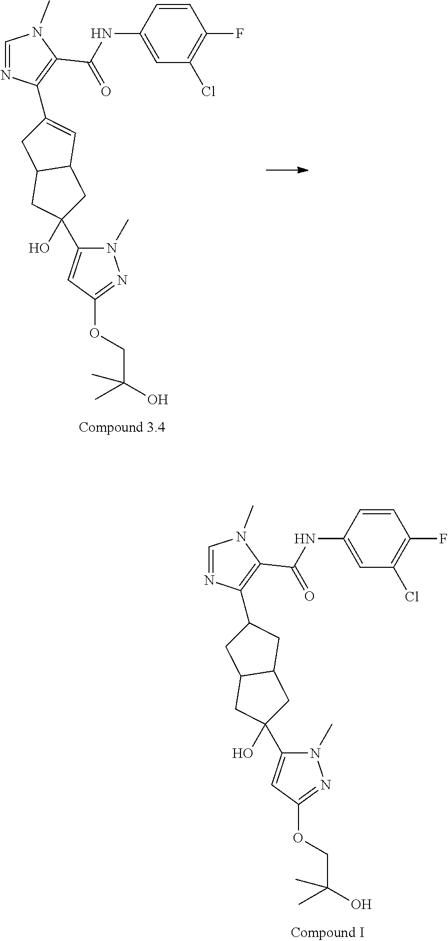 [SMILES: Cn1cnc(C2=CC3CC(O)(c4cc(OCC(C)(C)O)nn4C)CC3C2)c1C(=O)Nc1ccc(F)c(Cl)c1 ; Cn1cnc(C2CC3CC(O)(c4cc(OCC(C)(C)O)nn4C)CC3C2)c1C(=O)Nc1ccc(F)c(Cl)c1]

In an embodiment there is provided a method of synthesizing Compound Ia, or a salt thereof, comprising converting (such as hydrogenating) Compound 3.4a to Compound Ia, or a salt thereof:

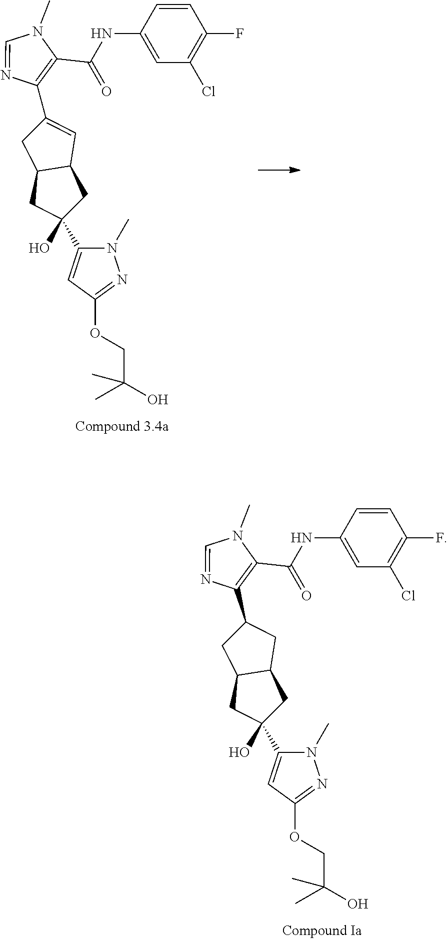 [SMILES: Cn1cnc(C2=C[C@H]3C[C@](O)(c4cc(OCC(C)(C)O)nn4C)C[C@H]3C2)c1C(=O)Nc1ccc(F)c(Cl)c1 ; Cn1cnc([C@@H]2C[C@@H]3C[C@@](O)(c4cc(OCC(C)(C)O)nn4C)C[C@@H]3C2)c1C(=O)Nc1ccc(F)c(Cl)c1]

In the hydrogenation reaction, suitably the catalyst is palladium on carbon, such as 10% palladium on carbon. Suitably the solvent in THF.

In an embodiment there is provided a method of synthesizing Compound Ia, or a salt thereof, comprising the steps of:
i. converting Compound VI to Compound 3.4a; and
ii. converting Compound 3.4a to Compound Ia, or a salt thereof,

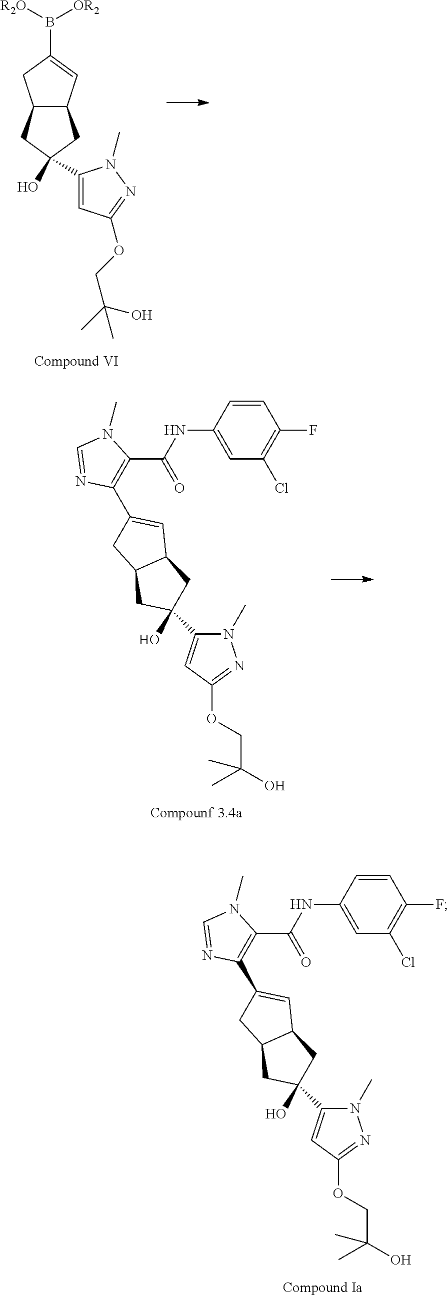

wherein each R₂ is independently hydrogen, alkyl or phenyl, or both are joined to form a cyclic boronic ester (such as pinacol, neopentyl or catechol boronic esters).

One embodiment of the present disclosure is a method for synthesizing Compound I, or a salt thereof, comprising the steps of:
i. converting Compound 3.3 to Compound 3.4; and
ii. converting Compound 3.4 to Compound I, or a salt thereof,

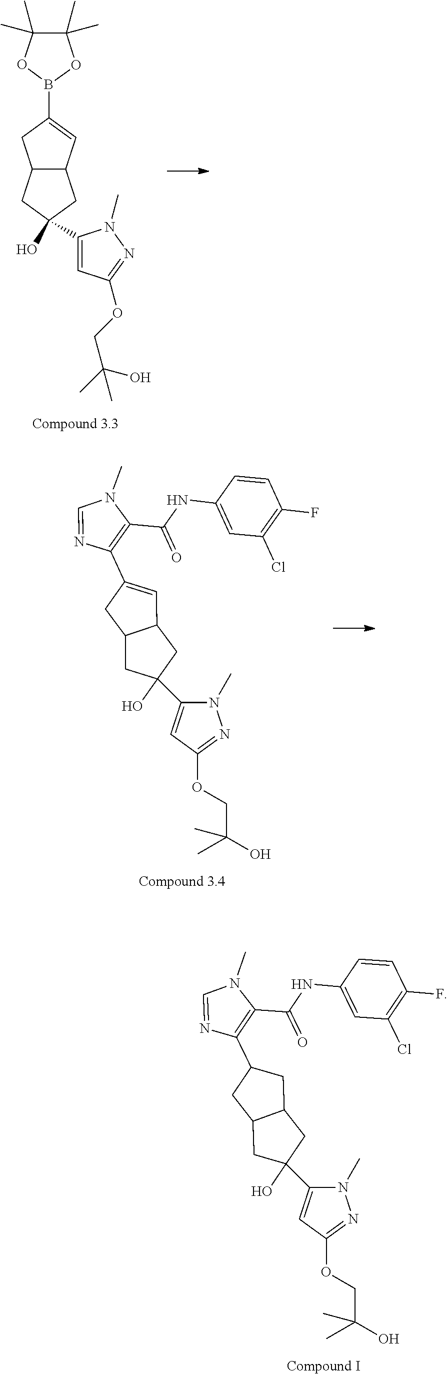 [SMILES: Cn1nc(OCC(C)(C)O)cc1[C@@]1(O)CC2C=C(B3OC(C)(C)C(C)(C)O3)CC2C1 ; Cn1cnc(C2=CC3CC(O)(c4cc(OCC(C)(C)O)nn4C)CC3C2)c1C(=O)Nc1ccc(F)c(Cl)c1 ; Cn1cnc(C2CC3CC(O)(c4cc(OCC(C)(C)O)nn4C)CC3C2)c1C(=O)Nc1ccc(F)c(Cl)c1]

In an embodiment there is provided a method of synthesizing Compound Ia, or a salt thereof, comprising the steps of:
i. converting Compound 3.3a to Compound 3.4a; and
ii. converting Compound 3.4a to Compound Ia, or a salt thereof,

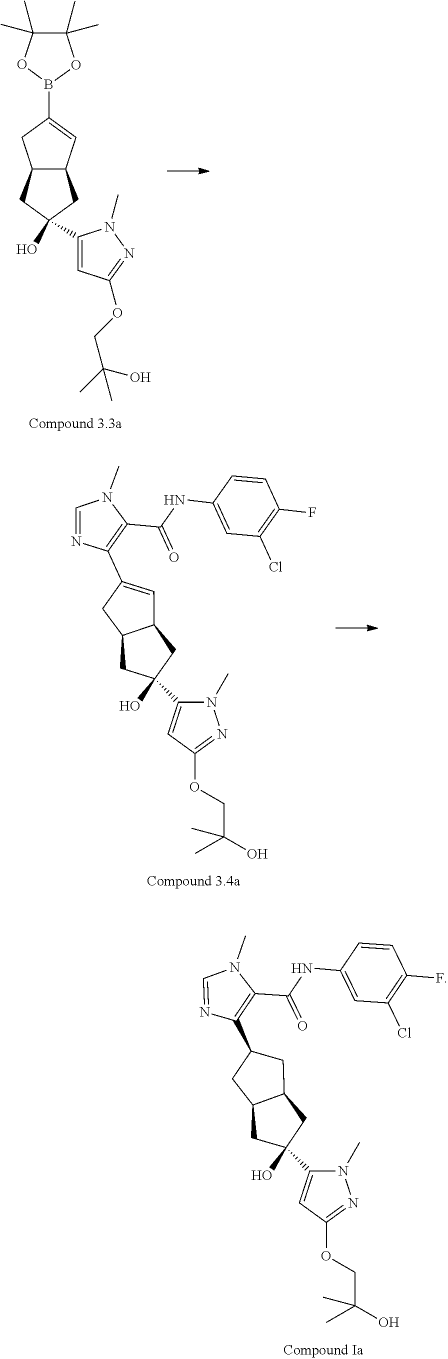 [SMILES: Cn1nc(OCC(C)(C)O)cc1[C@]1(O)C[C@H]2CC(B3OC(C)(C)C(C)(C)O3)=C[C@H]2C1 ; Cn1cnc(C2=C[C@H]3C[C@](O)(c4cc(OCC(C)(C)O)nn4C)C[C@H]3C2)c1C(=O)Nc1ccc(F)c(Cl)c1 ; Cn1cnc([C@@H]2C[C@@H]3C[C@@](O)(c4cc(OCC(C)(C)O)nn4C)C[C@@H]3C2)c1C(=O)Nc1ccc(F)c(Cl)c1]

In an embodiment there is provided a method of synthesizing Compound Ia, or a salt thereof, comprising the steps of:
i. converting Compound V to Compound VI;
ii. converting Compound VI to Compound 3.4a; and
iii. converting Compound 3.4a to Compound Ia, or a salt thereof,

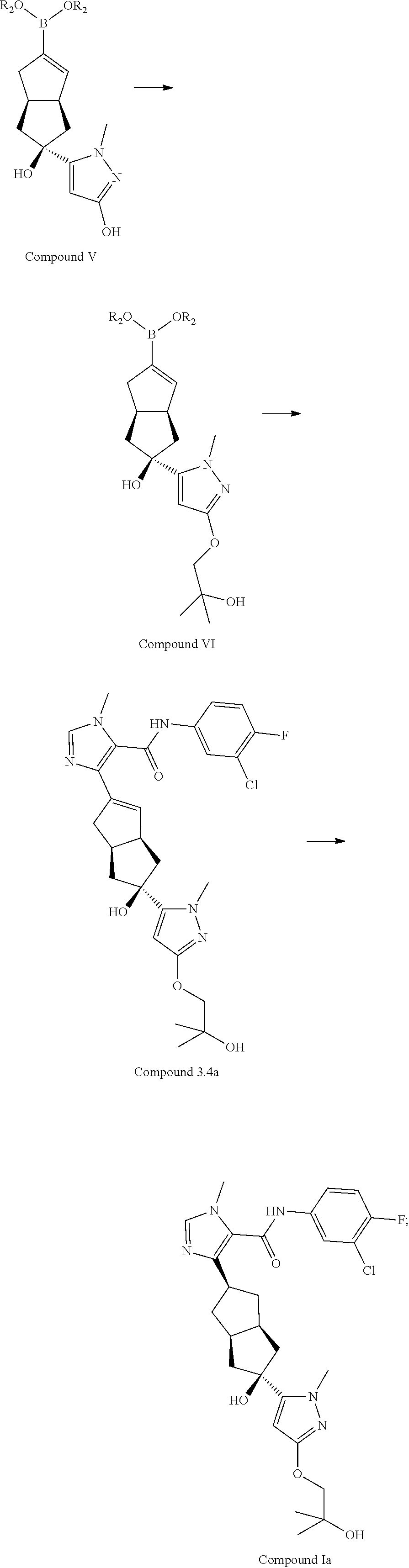

wherein each R₂ is independently hydrogen, alkyl or phenyl, or both are joined to form a cyclic boronic ester (such as pinacol, neopentyl or catechol boronic esters).

One embodiment of the present disclosure is a method for synthesizing Compound I, or a salt thereof, comprising the steps of:
i. converting Compound 3.2 to Compound 3.3;
ii. converting Compound 3.3 to Compound 3.4; and
iii. converting Compound 3.4 to Compound I, or a salt thereof,

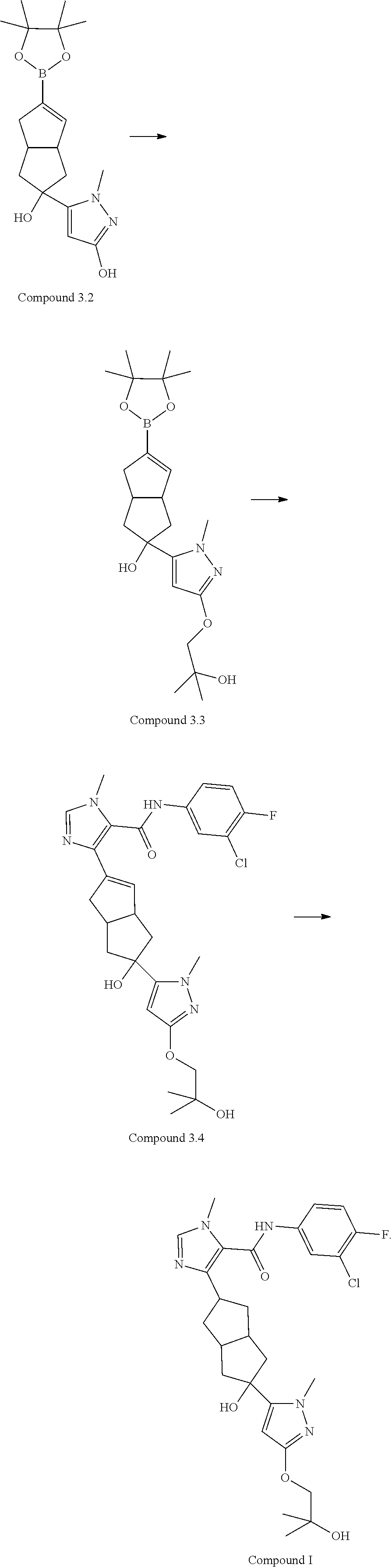 [SMILES: Cn1nc(O)cc1C1(O)CC2C=C(B3OC(C)(C)C(C)(C)O3)CC2C1 ; Cn1nc(OCC(C)(C)O)cc1C1(O)CC2C=C(B3OC(C)(C)C(C)(C)O3)CC2C1 ; Cn1cnc(C2=CC3CC(O)(c4cc(OCC(C)(C)O)nn4C)CC3C2)c1C(=O)Nc1ccc(F)c(Cl)c1 ; Cn1cnc(C2CC3CC(O)(c4cc(OCC(C)(C)O)nn4C)CC3C2)c1C(=O)Nc1ccc(F)c(Cl)c1]

In an embodiment there is provided a method of synthesizing Compound Ia, or a salt thereof, comprising the steps of:
i. converting Compound 3.2a to Compound 3.3a;
ii. converting Compound 3.3a to Compound 3.4a; and
iii. converting Compound 3.4a to Compound Ia, or a salt thereof,

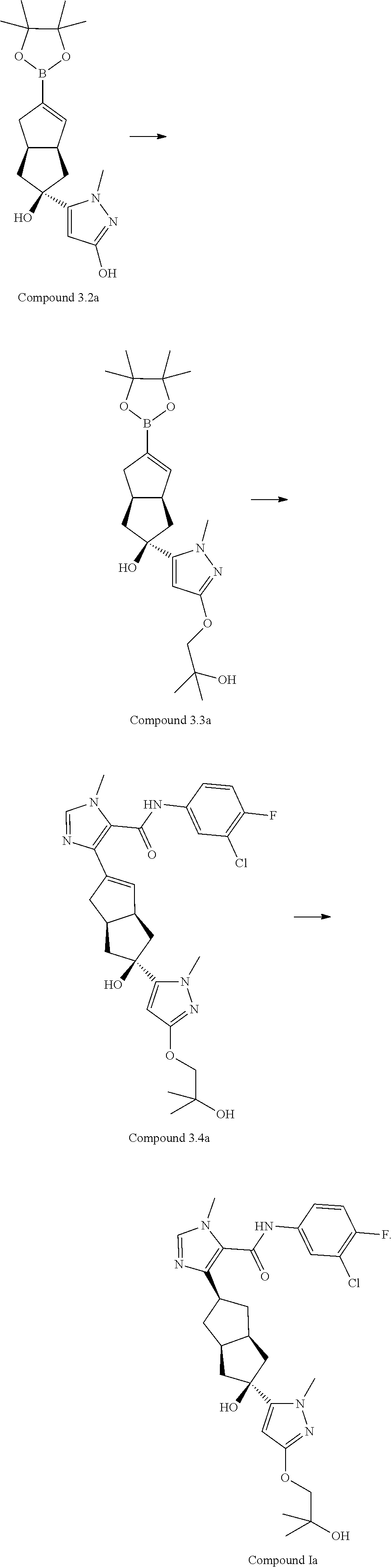 [SMILES: Cn1nc(O)cc1[C@]1(O)C[C@H]2CC(B3OC(C)(C)C(C)(C)O3)=C[C@H]2C1 ; Cn1nc(OCC(C)(C)O)cc1[C@]1(O)C[C@H]2CC(B3OC(C)(C)C(C)(C)O3)=C[C@H]2C1 ; Cn1cnc(C2=C[C@H]3C[C@](O)(c4cc(OCC(C)(C)O)nn4C)C[C@H]3C2)c1C(=O)Nc1ccc(F)c(Cl)c1 ; Cn1cnc([C@@H]2C[C@@H]3C[C@@](O)(c4cc(OCC(C)(C)O)nn4C)C[C@@H]3C2)c1C(=O)Nc1ccc(F)c(Cl)c1]

In an embodiment there is provided a method of synthesizing Compound Ia, or a salt thereof, comprising the steps of:
i. converting Compound IV to Compound V;
ii. converting Compound V to Compound VI;
iii. converting Compound VI to Compound 3.4a; and
iv. converting Compound 3.4a to Compound Ia, or a salt thereof,

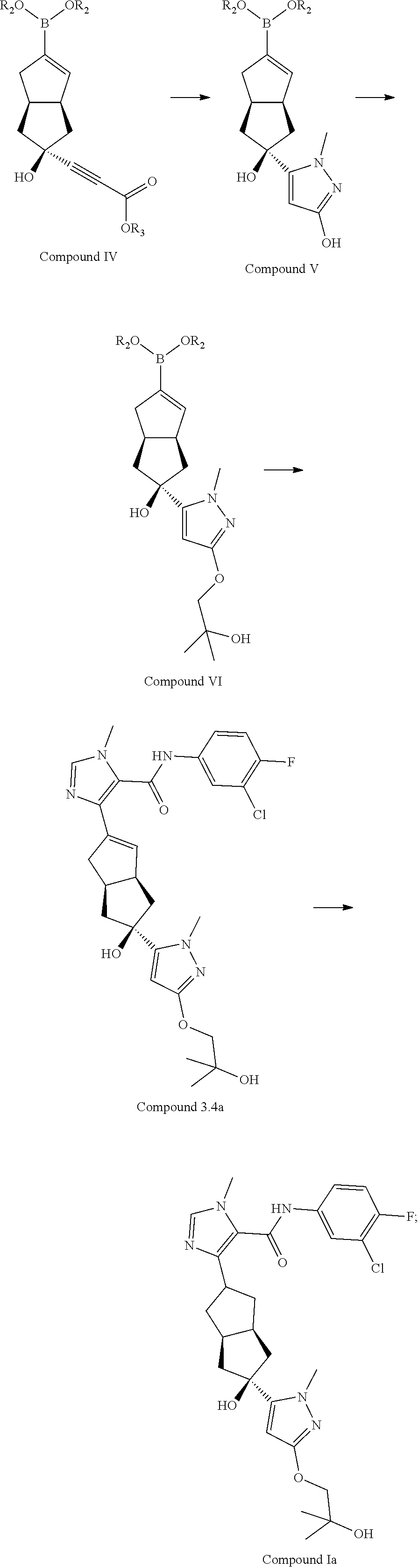

wherein each R₂ is independently hydrogen, alkyl or phenyl, or both are joined to form a cyclic boronic ester (such as pinacol, neopentyl or catechol boronic esters); and R₃ is C₁₋₄alkyl (such as methyl or ethyl).

One embodiment of the present disclosure is a method for synthesizing Compound I, or a salt thereof, comprising the steps of:
i. converting Compound 3.1 to Compound 3.2;
ii. converting Compound 3.2 to Compound 3.3;
iii. converting Compound 3.3 to Compound 3.4; and
iv. converting Compound 3.4 to Compound I, or a salt thereof.

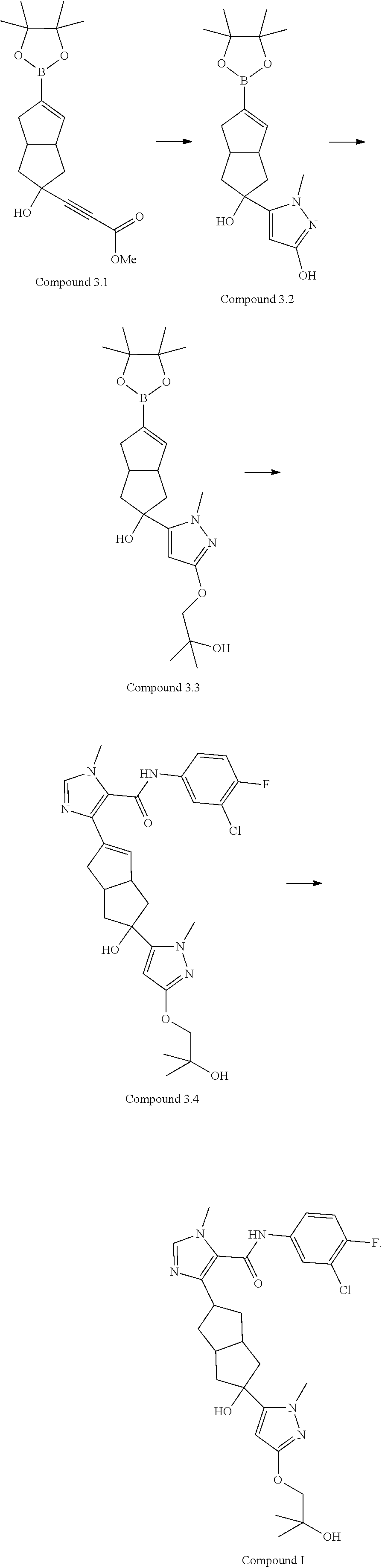 [SMILES: COC(=O)C#CC1(O)CC2C=C(B3OC(C)(C)C(C)(C)O3)CC2C1 ; Cn1nc(O)cc1C1(O)CC2C=C(B3OC(C)(C)C(C)(C)O3)CC2C1 ; Cn1nc(OCC(C)(C)O)cc1C1(O)CC2C=C(B3OC(C)(C)C(C)(C)O3)CC2C1 ; Cn1cnc(C2=CC3CC(O)(c4cc(OCC(C)(C)O)nn4C)CC3C2)c1C(=O)Nc1ccc(F)c(Cl)c1 ; Cn1cnc(C2CC3CC(O)(c4cc(OCC(C)(C)O)nn4C)CC3C2)c1C(=O)Nc1ccc(F)c(Cl)c1]

In an embodiment there is provided a method of synthesizing Compound Ia, or a salt thereof, comprising the steps of:
i. converting Compound 3.1a to Compound 3.2a;
ii. converting Compound 3.2a to Compound 3.3a;
iii. converting Compound 3.3a to Compound 3.4a; and
iv. converting Compound 3.4a to Compound Ia, or a salt thereof,

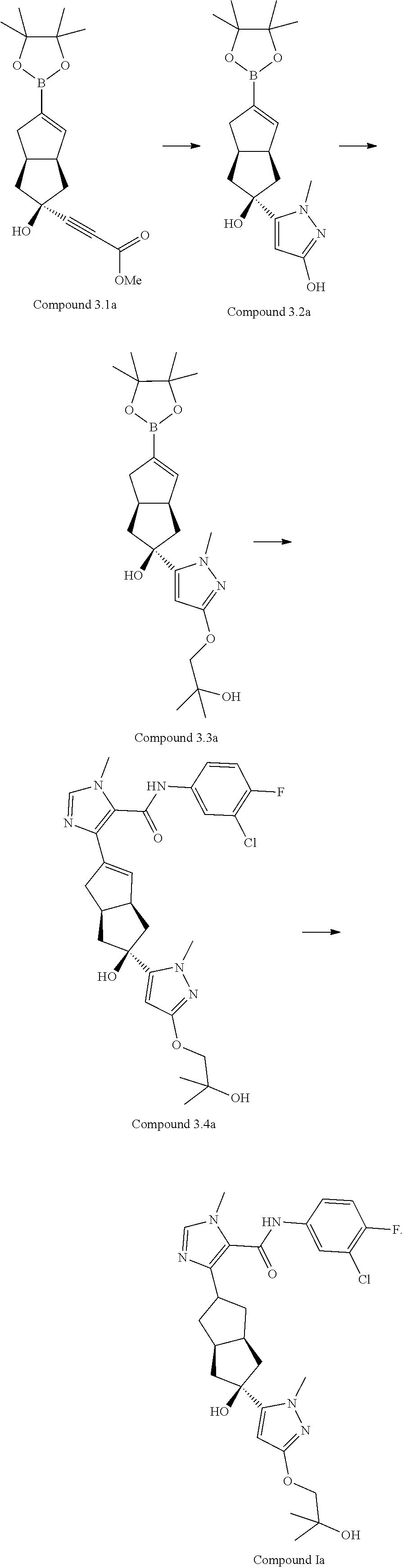 [SMILES: COC(=O)C#C[C@]1(O)C[C@H]2CC(B3OC(C)(C)C(C)(C)O3)=C[C@H]2C1 ; Cn1nc(O)cc1[C@]1(O)C[C@H]2CC(B3OC(C)(C)C(C)(C)O3)=C[C@H]2C1 ; Cn1nc(OCC(C)(C)O)cc1[C@]1(O)C[C@H]2CC(B3OC(C)(C)C(C)(C)O3)=C[C@H]2C1 ; Cn1cnc(C2=C[C@H]3C[C@](O)(c4cc(OCC(C)(C)O)nn4C)C[C@H]3C2)c1C(=O)Nc1ccc(F)c(Cl)c1 ; Cn1cnc(C2C[C@@H]3C[C@@](O)(c4cc(OCC(C)(C)O)nn4C)C[C@@H]3C2)c1C(=O)Nc1ccc(F)c(Cl)c1]

In an embodiment there is provided a method of synthesizing Compound a, or a salt thereof, comprising the steps of:
i. converting Compound III to Compound IV;
ii. converting Compound IV to Compound V;
iii. converting Compound V to Compound VI;
iv. converting Compound VI to Compound 3.4a; and
v. converting Compound 3.4a to Compound Ia, or a salt thereof,

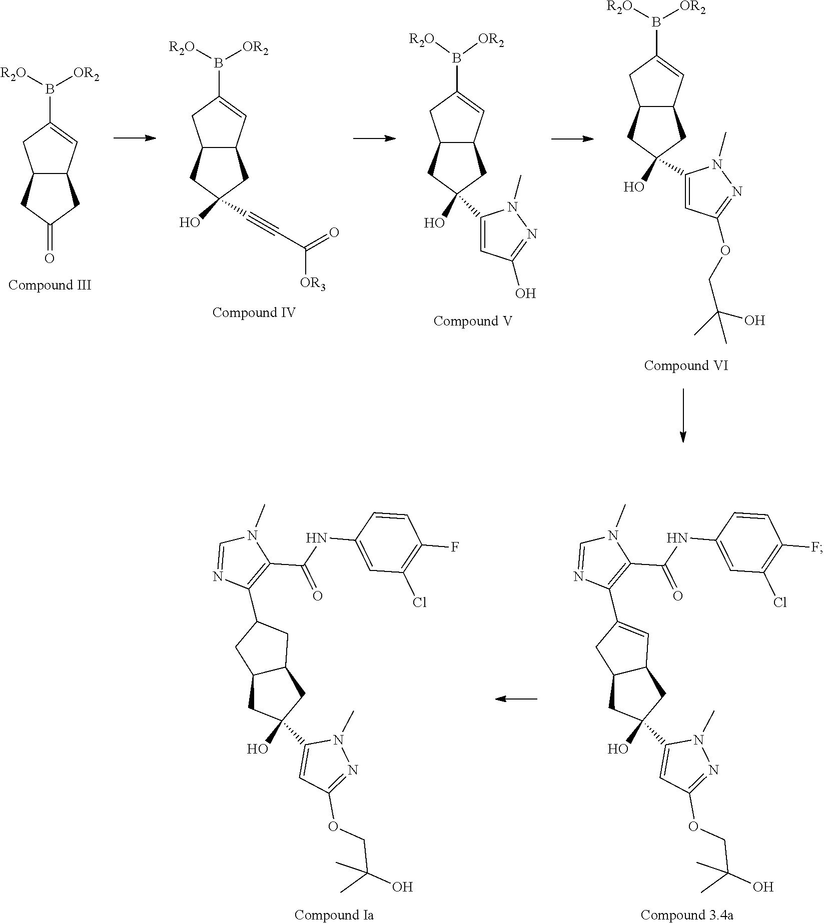

wherein each R₂ is independently hydrogen, alkyl or phenyl, or both are joined to form a cyclic boronic ester (such as pinacol, neopentyl or catechol boronic esters); and R₃ is C₁₋₄alkyl (such as methyl or ethyl).

One embodiment of the present disclosure is a method for synthesizing Compound I, or a salt thereof, comprising the steps of:
i. converting Compound 3 to Compound 3.1;
ii. converting Compound 3.1 to Compound 3.2;
iii. converting Compound 3.2 to Compound 3.3;
iv. converting Compound 3.3 to Compound 3.4; and
v. converting Compound 3.4 to Compound I, or a salt thereof,

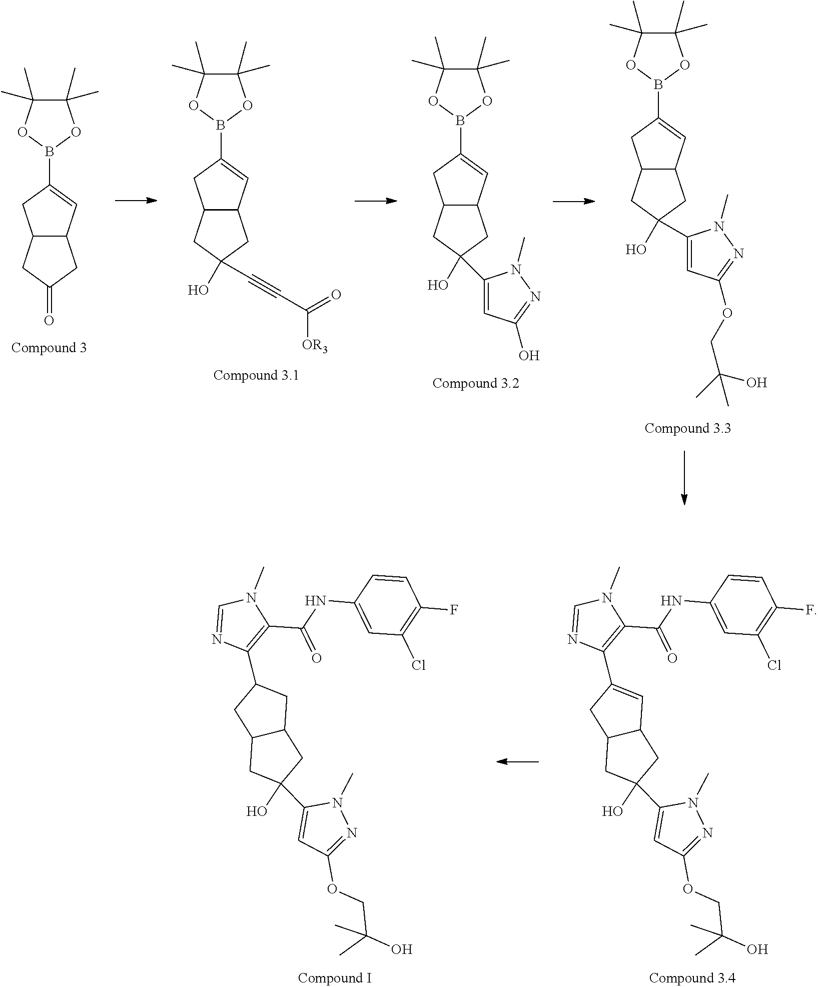

In an embodiment there is provided a method of synthesizing Compound Ia, or a salt thereof, comprising the steps of:
i. converting Compound 3a to Compound 3.1a;
ii. converting Compound 3.1a to Compound 3.2a;
iii. converting Compound 3.2a to Compound 3.3a;
iv. converting Compound 3.3a to Compound 3.4a; and
v. converting Compound 3.4a to Compound Ia, or a salt thereof,

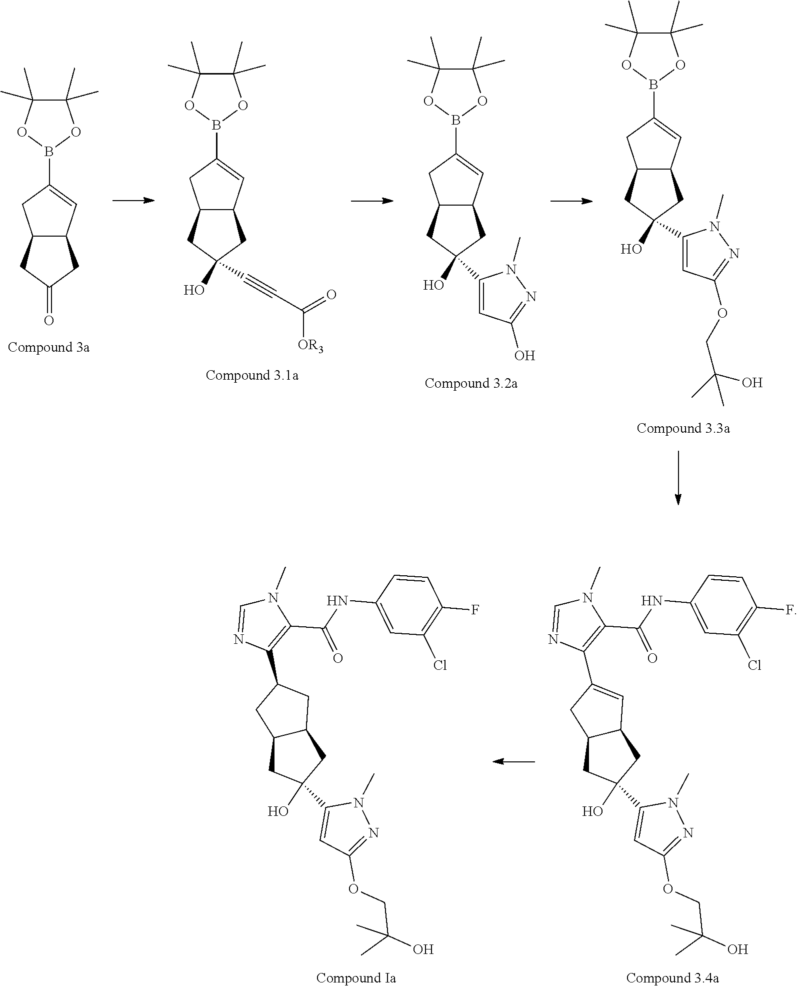

### Alternative Route to Intermediate Compound 3.4

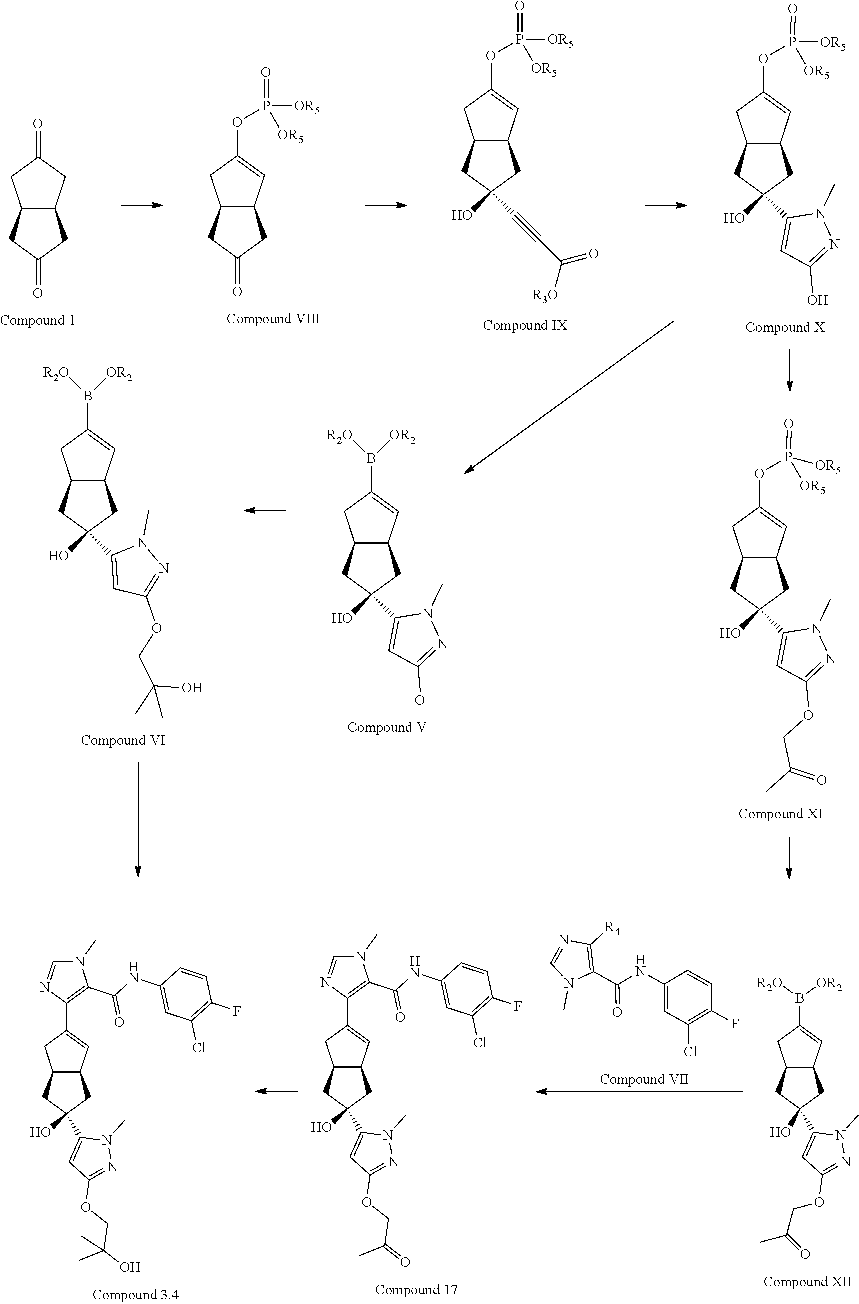

The above alternative route intersects with the process described herein at either Compound V or Compound 3.4. Examples 4-6 below are embodiments of this alternative route, which starts from Compound 1 (tetrahydropentalene-2,5(1H,3H)-dione).

Compound VIII, where R₅ is C₁₋₄alkyl or phenyl (such as phenyl), may be formed by reacting Compound 1 with a suitable strong base (such as butyl lithium or lithium hexamethyldisilazane), followed by addition of X—P(O)(OR₅)₂ where R₅ is C₁₋₄alkyl or phenyl and X is a suitable leaving group such as chloro or bromo. Suitably, in Compound VIII R₅ are both phenyl (Compound 12), or R₅ are both ethyl (Compound 12.1).

Compound IX wherein R₃ is C₁₋₄alkyl (such as methyl or ethyl), may be formed by reacting Compound VIII with an organometallic formed from the reaction of C₁₋₄alkyl propiolate with a suitable strong base (such as butyl lithium or lithium hexamethyldisilazane). Suitably, in Compound IX R₅ are both phenyl and R₃ is methyl (Compound 13).

Compound X may be formed by reacting Compound IX with methyl hydrazine or a salt of methyl hydrazine (such as methylhydrazine sulfate) in a suitable solvent (such as toluene) and with heating (such as at >50° C., >60° C., >70° C., or about 80° C.). If a salt of methyl hydrazine is used, then a suitable base (such as triethylamine, or DIPEA) is also required in the reaction to break the salt. Suitably, in Compound X R₅ are both phenyl (Compound 14).

Compound V, wherein each R₂ is independently hydrogen, alkyl or phenyl, or both are joined to form a cyclic boronic ester (such as pinacol, neopentyl or catechol boronic esters), may be formed by reacting Compound X with a suitable boron reagent (such as bis(pinacolato)diboron) in the presence of a palladium catalyst (such as Pd(XPhos)allylCl) and a suitable base (such as potassium pivalate). Suitably, in Compound V both R₂ are joined to form a pinacol boronic ester (Compound 3.2). Compound V may then be converted to Compound VI and Compound VI may be converted to Compound 3.4 by the methods described hereinabove.

Compound XI may be formed by reacting Compound X with a suitable base (such as potassium carbonate) followed by addition of a suitable alkylating agent (such as chloroacetone). Suitably, in Compound XI R₅ are both phenyl (Compound 15).

Compound XII, wherein each R₂ is independently hydrogen, alkyl or phenyl, or both are joined to form a cyclic boronic ester (such as pinacol, neopentyl or catechol boronic esters), may be formed by reacting Compound XI with a suitable boron reagent (such as bis(pinacolato)diboron) in the presence of a palladium catalyst (such as Pd(XPhos)allylCl) and a suitable base (such as potassium pivalate). Suitably, in Compound XII both R₂ are joined to form a pinacol boronic ester (Compound 16).

Compound 17 may be formed by carrying out a cross-coupling reaction between Compound XII and Compound VII, wherein R₄ is a suitable leaving group (such as bromo, iodo, or triflate). The cross-coupling reaction proceeds in the presence of a suitable base (such as potassium carbonate, cesium carbonate of potassium acetate) and a suitable palladium catalyst (such as Pd(PPh₃)₄, Pd(dppf)Cl₂, or cataCXium® A Pd G3—mesylate[(di(1-adamantyl)-n-butylphosphine)-2-(2′-amino-1,1′-biphenyl)]-palladium(II)) with heating (such as at >50° C., >60° C., >70° C., or about 80° C.) in a suitable solvent (such as dioxane, DMA, NMP and/or water).

Compound 3.4 may be formed by reacting Compound 17 with a suitable methyl anion species (such as methyl magnesium bromide) in a suitable solvent (such as THF).

In one aspect of the present disclosure there is provided a method for synthesizing Compound Ia, or a salt thereof, comprising the steps of:
i. converting Compound 17 to Compound 3.4a; and
ii. converting Compound 3.4a to Compound Ia, or a salt thereof,

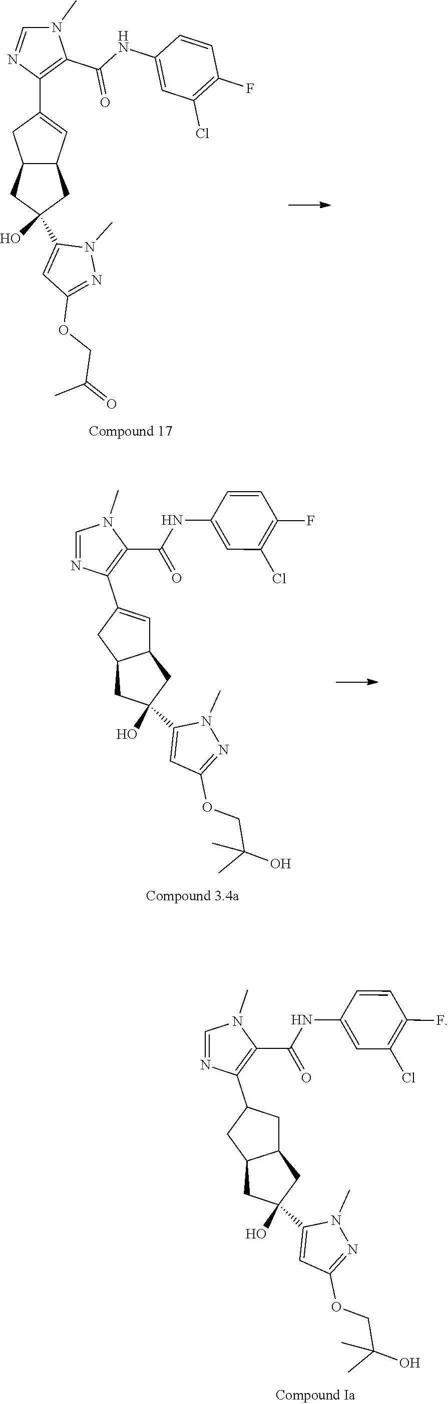 [SMILES: CC(=O)COc1cc([C@]2(O)C[C@H]3CC(c4ncn(C)c4C(=O)Nc4ccc(F)c(Cl)c4)=C[C@H]3C2)n(C)n1 ; Cn1cnc(C2=C[C@H]3C[C@](O)(c4cc(OCC(C)(C)O)nn4C)C[C@H]3C2)c1C(=O)Nc1ccc(F)c(Cl)c1 ; Cn1cnc(C2C[C@@H]3C[C@@](O)(c4cc(OCC(C)(C)O)nn4C)C[C@@H]3C2)c1C(=O)Nc1ccc(F)c(Cl)c1]

One embodiment of the present disclosure is a method for synthesizing Compound Ia, or a salt thereof, comprising the steps of:
i. converting Compound 16 to Compound 17;
ii. converting Compound 17 to Compound 3.4a; and
iii. converting Compound 3.4a to Compound Ia, or a salt thereof,

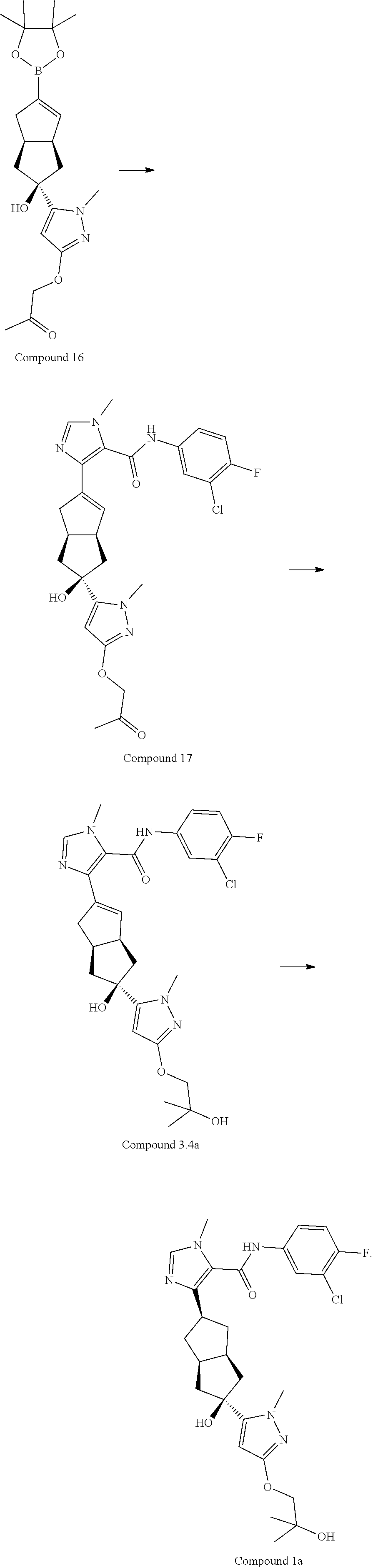

One embodiment of the present disclosure is a method for synthesizing Compound Ia, or a salt thereof, comprising the steps of:
i. converting Compound 15 to Compound 16;
ii. converting Compound 16 to Compound 17;
iii. converting Compound 17 to Compound 3.4a; and
iv. converting Compound 3.4a to Compound Ia, or a salt thereof,

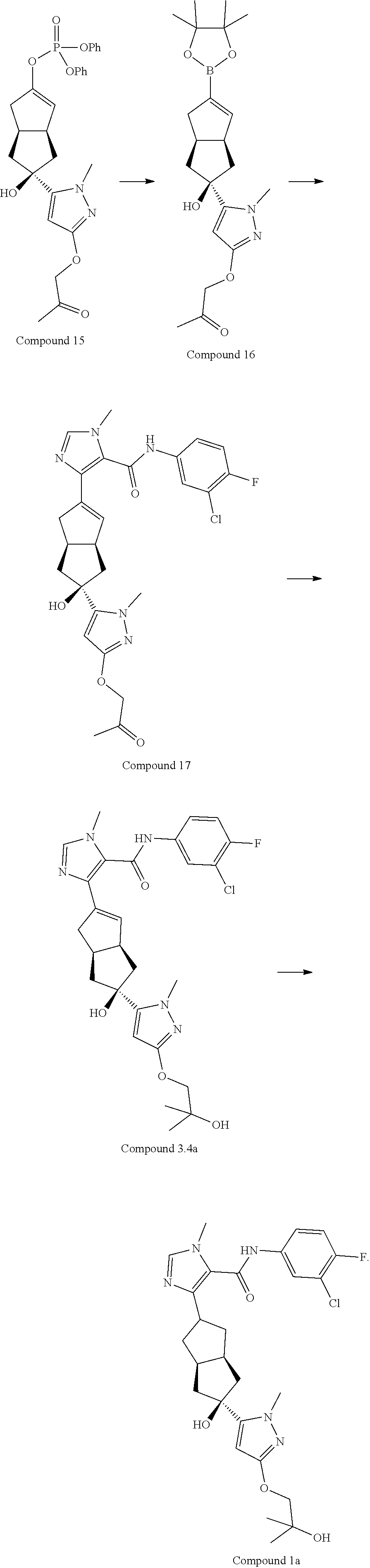

One embodiment of the present disclosure is a method for synthesizing Compound I, or a salt thereof, comprising the steps of:
i. converting Compound 14 to Compound 15;
ii. converting Compound 15 to Compound 16;
iii. converting Compound 16 to Compound 17;
iv. converting Compound 17 to Compound 3.4a; and
v. converting Compound 3.4a to Compound Ia, or a salt thereof,

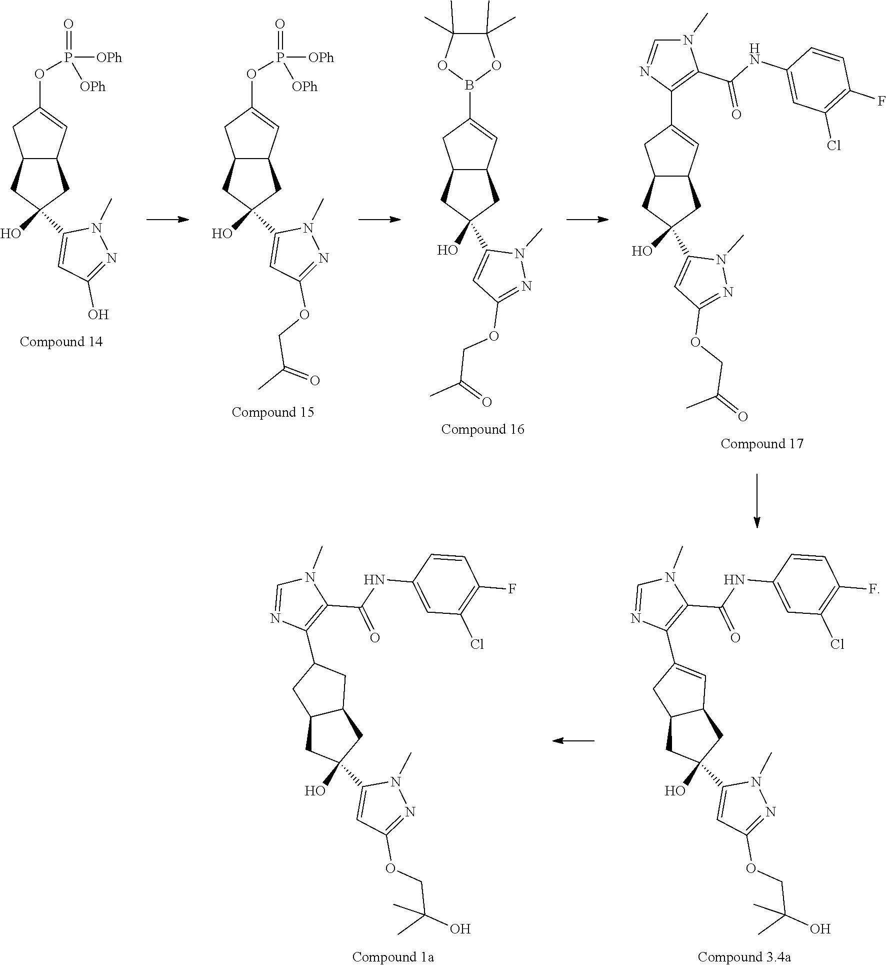

In one aspect of the present disclosure there is provided a method for synthesizing Compound Ia, or a salt thereof, comprising the steps of:
i. converting Compound 14 to Compound 3.2a;
ii. converting Compound 3.2a to Compound 3.3a;
iii. converting Compound 3.3a to Compound 3.4a; and
iv. converting Compound 3.4a to Compound Ia, or a salt thereof,

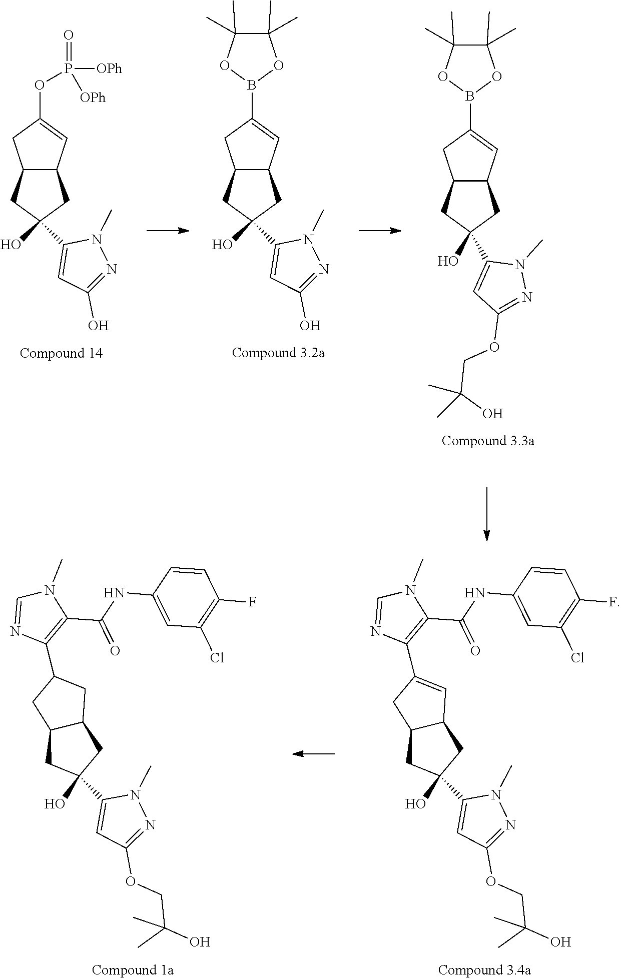

In one aspect of the present disclosure there is provided a method for synthesizing Compound 14, comprising the step of converting Compound 13 to Compound 14;

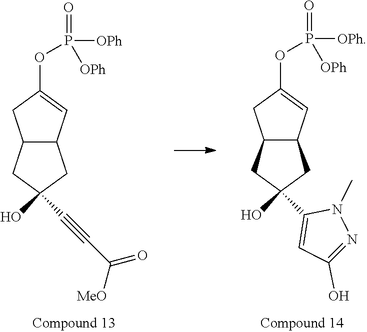 [SMILES: COC(=O)C#C[C@@]1(O)CC2C=C(OP(=O)(Oc3ccccc3)Oc3ccccc3)CC2C1 ; Cn1nc(O)cc1[C@]1(O)C[C@H]2CC(OP(=O)(Oc3ccccc3)Oc3ccccc3)=C[C@H]2C1]

In one embodiment there is provided a method for synthesizing Compound 14, comprising the steps of:
i. converting Compound 12 to Compound 13; and
ii. converting Compound 13 to Compound 14;

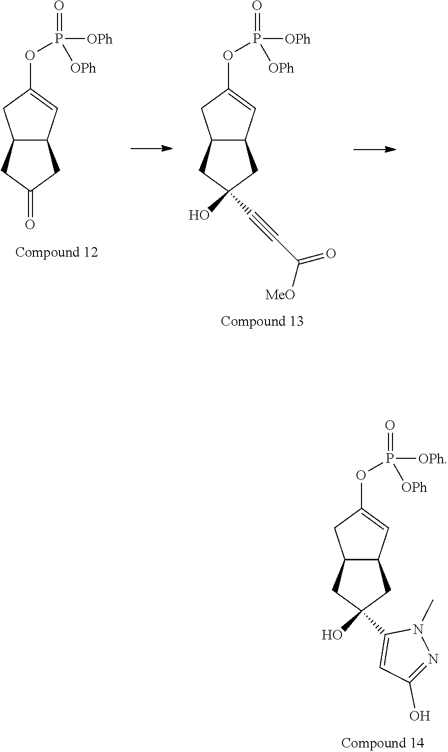 [SMILES: O=C1C[C@H]2CC(OP(=O)(Oc3ccccc3)Oc3ccccc3)=C[C@H]2C1 ; COC(=O)C#C[C@]1(O)C[C@H]2CC(OP(=O)(Oc3ccccc3)Oc3ccccc3)=C[C@H]2C1 ; Cn1nc(O)cc1[C@]1(O)C[C@H]2CC(OP(=O)(Oc3ccccc3)Oc3ccccc3)=C[C@H]2C1]

In one embodiment there is provided a method for synthesizing Compound 14, comprising the steps of:
i. converting Compound 1 to Compound 12;
ii. converting Compound 12 to Compound 13; and
iii. converting Compound 13 to Compound 14;

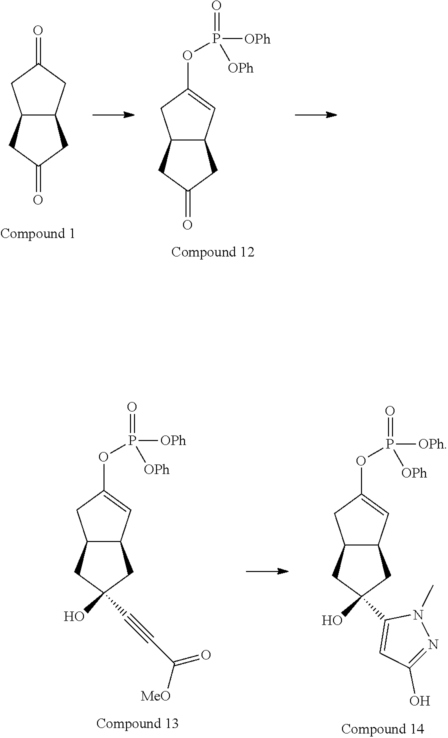 [SMILES: O=C1C[C@H]2CC(=O)C[C@H]2C1 ; O=C1C[C@H]2CC(OP(=O)(Oc3ccccc3)Oc3ccccc3)=C[C@H]2C1 ; COC(=O)C#C[C@]1(O)C[C@H]2CC(OP(=O)(Oc3ccccc3)Oc3ccccc3)=C[C@H]2C1 ; Cn1nc(O)cc1[C@]1(O)C[C@H]2CC(OP(=O)(Oc3ccccc3)Oc3ccccc3)=C[C@H]2C1]

### Intermediates

In another aspect, the present invention relates to intermediates which are useful in the preparation of Compound I.

In one embodiment of this aspect, there is provided the intermediate Compound II:

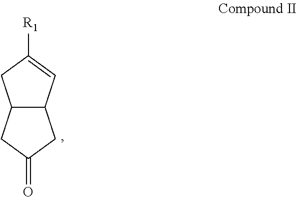

wherein R₁ is chloro, bromo, iodo, triflate, mesylate or tosylate.

In one embodiment, there is provided the intermediate Compound III:

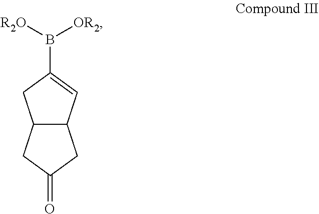

wherein each R₂ is independently hydrogen, alkyl or phenyl, or both R₂ are joined to form a cyclic boronic ester.

In one embodiment, there is provided the intermediate Compound IV:

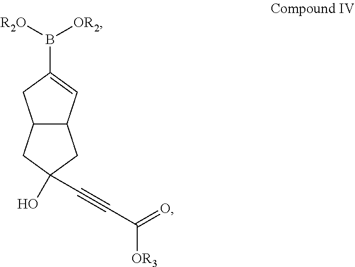

wherein each R₂ is independently hydrogen, alkyl or phenyl, or both R₂ are joined to form a cyclic boronic ester, and R₃ is C₁₋₄alkyl. In an embodiment, both R₂ are joined to form a cyclic boronic ester, and R₃ is methyl. In an embodiment, both R₂ are joined to form a pinacol boronic ester, and R₃ is methyl. Suitably, Compound IV is methyl 3-(2-hydroxy-5-(4,4,5,5-tetramethyl-1,3,2-dioxaborolan-2-yl)-1,2,3,3a,4,6a-hexahydropentalen-2-yl)propiolate.

In one embodiment, there is provided Compound 3.1, or Compound 3.1a:

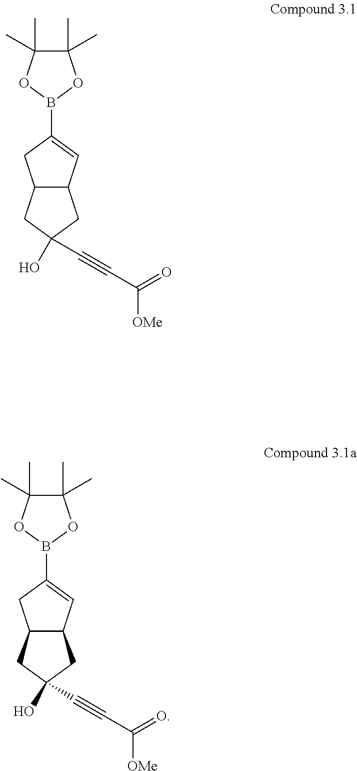 [SMILES: COC(=O)C#CC1(O)CC2C=C(B3OC(C)(C)C(C)(C)O3)CC2C1 ; COC(=O)C#C[C@]1(O)C[C@H]2CC(B3OC(C)(C)C(C)(C)O3)=C[C@H]2C1]

In one embodiment, there is provided the intermediate Compound V:

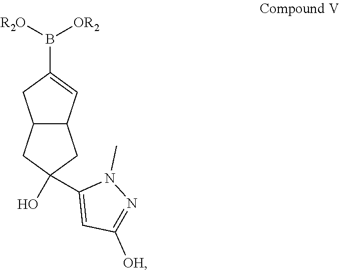

wherein each R₂ is independently hydrogen, alkyl or phenyl, or both R₂ are joined to form a cyclic boronic ester. In an embodiment, both R₂ are joined to form a cyclic boronic ester. In an embodiment, both R₂ are joined to form a pinacol boronic ester. Suitably, Compound V is 5-(2-hydroxy-5-(4,4,5,5-tetramethyl-1,3,2-dioxaborolan-2-yl)-1,2,3,3a,4,6a-hexahydropentalen-2-yl)-1-methyl-1H-pyrazol-3-ol.

In one embodiment, there is provided Compound 3.2, or Compound 3.2a:

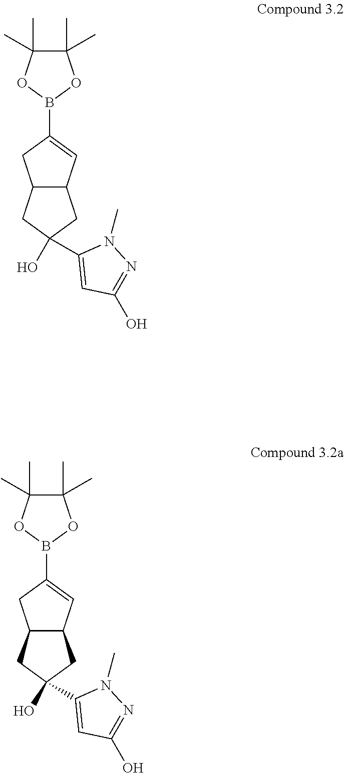 [SMILES: Cn1nc(O)cc1C1(O)CC2C=C(B3OC(C)(C)C(C)(C)O3)CC2C1 ; Cn1nc(O)cc1[C@]1(O)C[C@H]2CC(B3OC(C)(C)C(C)(C)O3)=C[C@H]2C1]

In one embodiment, there is provided the intermediate Compound VI:

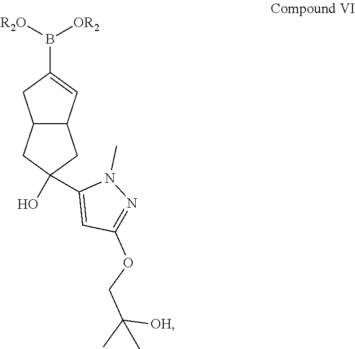

wherein each R₂ is independently hydrogen, alkyl or phenyl, or both R₂ are joined to form a cyclic boronic ester. In an embodiment, both R₂ are joined to form a cyclic boronic ester. In an embodiment, both R₂ are joined to form a pinacol boronic ester. Suitably, Compound VI is 2-(3-(2-hydroxy-2-methylpropoxy)-1-methyl-1H-pyrazol-5-yl)-5-(4,4,5,5-tetramethyl-1,3,2-dioxaboralan-2-yl)-1,2,3,3a,4,6a-hexahydropentalen-2-ol.

In one embodiment, there is provided Compound 3.3, or Compound 3.3a:

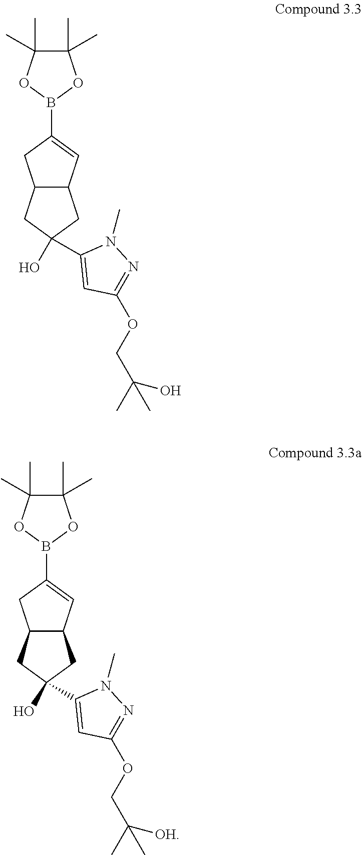 [SMILES: Cn1nc(OCC(C)(C)O)cc1C1(O)CC2C=C(B3OC(C)(C)C(C)(C)O3)CC2C1 ; Cn1nc(OCC(C)(C)O)cc1[C@]1(O)C[C@H]2CC(B3OC(C)(C)C(C)(C)O3)=C[C@H]2C1]

In one embodiment, there is provided the intermediate Compound 3.4:

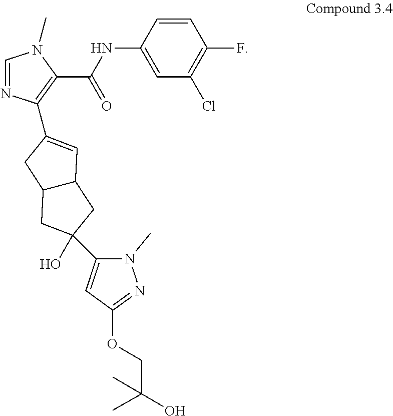 [SMILES: Cn1cnc(C2=CC3CC(O)(c4cc(OCC(C)(C)O)nn4C)CC3C2)c1C(=O)Nc1ccc(F)c(Cl)c1]

Compound 3.4 is N-(4-chloro-3-fluorophenyl)-4-(5-hydroxy-5-(3-(2-hydroxy-2-methylpropoxy)-1-methyl-1H-pyrazol-5-yl)-1,3a,4,5,6,6a-hexahydropentalen-2-yl)-1-methyl-1H-imidazole-5-carboxamide.

In one embodiment, there is provided the intermediate Compound 3.4a:

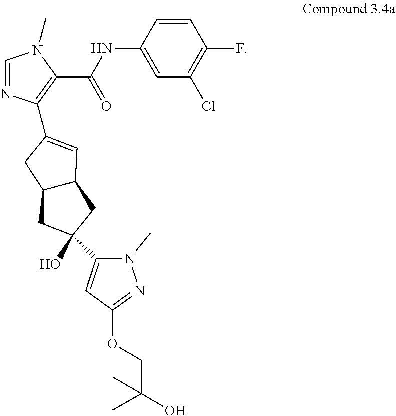 [SMILES: Cn1cnc(C2=C[C@H]3C[C@](O)(c4cc(OCC(C)(C)O)nn4C)C[C@H]3C2)c1C(=O)Nc1ccc(F)c(Cl)c1]

Compound 3.4a is N-(3-chloro-4-fluorophenyl)-4-((3aS,5S,6aR)-5-hydroxy-5-(3-(2-hydroxy-2-methylpropoxy)-1-methyl-1H-pyrazol-5-yl)-1,3a,4,5,6,6a-hexahydropentalen-2-yl)-1-methyl-1H-imidazole-5-carboxamide.

In one embodiment, there is provided the intermediate Compound VIII:

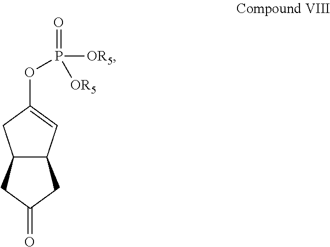

wherein R₅ is C₁₋₄alkyl or phenyl. In an embodiment, R₅ is ethyl or phenyl. In an embodiment, R₅ is phenyl. Suitably, Compound VIII is 5-oxo-1,3a,4,5,6,6a-hexahydropentalen-2-yl diphenyl phosphate.

In one embodiment, there is provided the intermediate Compound IX:

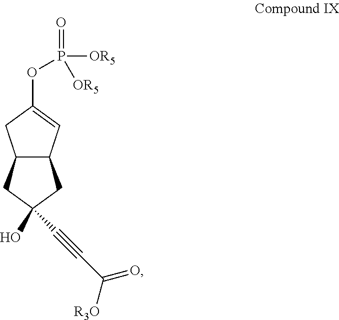

wherein R₅ is C₁₋₄alkyl or phenyl, and R₃ is C₁₋₄alkyl. In an embodiment, R₅ is ethyl or phenyl, and R₃ is methyl or ethyl. In an embodiment, R₅ is phenyl and R₃ is methyl. Suitably, Compound IX is methyl 3-(5-((diphenoxyphosphoryl)oxy)-2-hydroxy-1,2,3,3a,4,6a-hexahydropentalen-2-yl)propiolate.

In one embodiment, there is provided the intermediate Compound X:

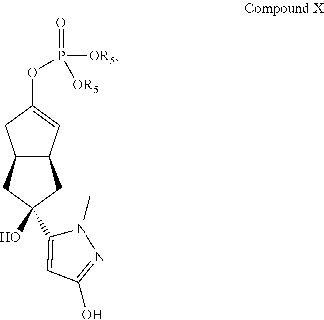

wherein R₅ is C₁₋₄alkyl or phenyl. In an embodiment, R₅ is ethyl or phenyl. In an embodiment, R₅ is phenyl. Suitably, Compound X is 5-hydroxy-5-(3-hydroxy-1-methyl-1H-pyrazol-5-yl)-1,3a,4,5,6,6a-hexahydropentalen-2-yl diphenyl phosphate.

In one embodiment, there is provided the intermediate Compound XI:

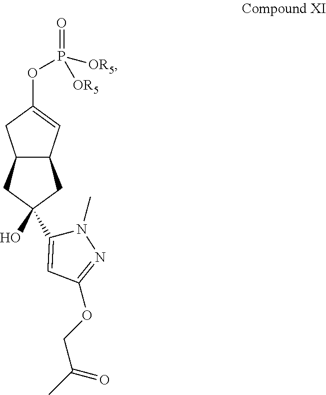

wherein R₅ is C₁₋₄alkyl or phenyl. In an embodiment, R₅ is ethyl or phenyl. In an embodiment, R₅ is phenyl. Suitably, Compound XI is 5-hydroxy-5-(1-methyl-3-(2-oxopropoxy)-1H-pyrazol-5-yl)-1,3a,4,5,6,6a-hexahydropentalen-2-yl diphenyl phosphate.

In one embodiment, there is provided the intermediate Compound XII:

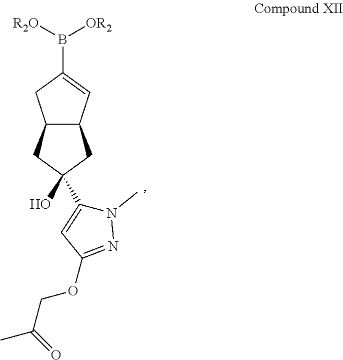

wherein each R₂ is independently hydrogen, alkyl or phenyl, or both are joined to form a cyclic boronic ester. In an embodiment, R₂ are both joined to form a cyclic pinacol, neopentyl or catechol boronic esters. In an embodiment, R₂ are both joined to form a cyclic pinacol boronic ester. Suitably, Compound XII is 1-((5-(2-hydroxy-5-(4,4,5,5-tetramethyl-1,3,2-dioxaborolan-2-yl)-1,2,3,3a,4,6a-hexahydropentalen-2-yl)-1-methyl-1H-pyrazol-3-yl)oxy)propan-2-one.

In one embodiment, there is provided the intermediate Compound 17:

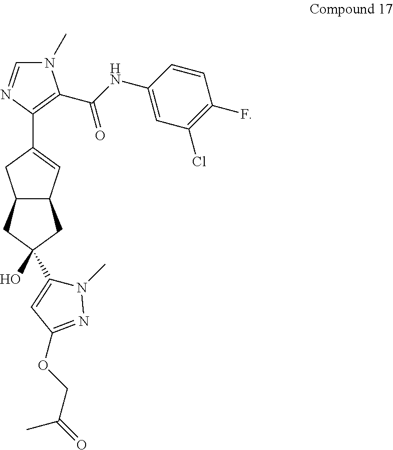 [SMILES: CC(=O)COc1cc([C@]2(O)C[C@H]3CC(c4ncn(C)c4C(=O)Nc4ccc(F)c(Cl)c4)=C[C@H]3C2)n(C)n1]

Compound 17 is N-(4-chloro-3-fluorophenyl)-4-(5-hydroxy-5-(1-methyl-3-(2-oxopropoxy)-1H-pyrazol-5-yl)-1,3a,4,5,6,6a-hexahydropentalen-2-yl)-1-methyl-1H-imidazole-5-carboxamide.

### EXAMPLES

As previously noted, the present disclosure relates to alternative and novel synthetic methods for the compounds disclosed in PCT/US2021/028323 (PCT '323), which is hereby incorporated by reference in its entirety.

One route to obtain Compound I uses the guidance of PCT '323 as a template:

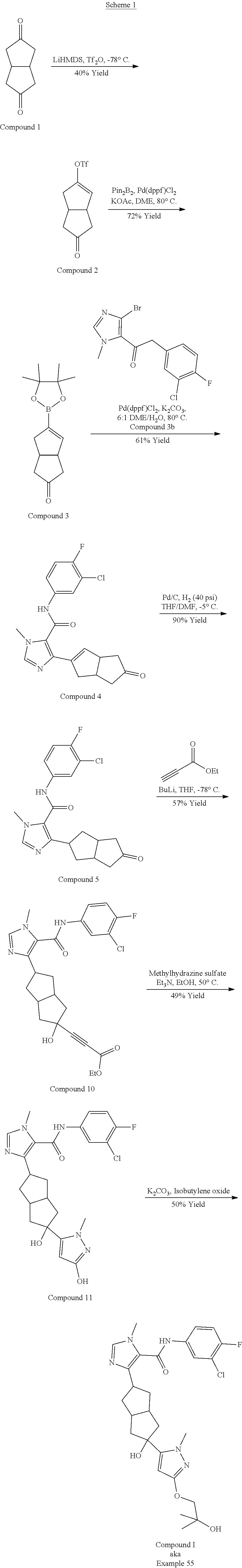

As shown in Scheme 1, one route offers the introduction of the starting material referred to as Compound 3b (N-(4-chloro-3-fluorophenyl)-4-bromo-1-methyl-1H-imidazole-5-carboxamide) early in the overall route, namely at the third step in the process.

The present disclosure includes an alternative route that delays the introduction of the starting material Compound 3b until later in the synthetic scheme, thereby saving on costs and reducing waste.

Therefore, one embodiment of the present disclosure provides the following route shown in Scheme 2:

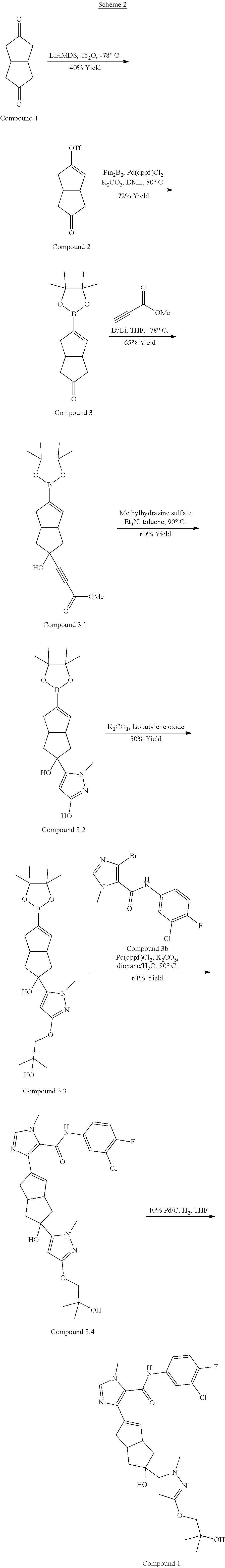

As shown in Scheme 2, the Compound 3b starting material is introduced at step 6 of this embodiment of the present disclosure, and just prior to the penultimate step to produce the Product, Compound I.

### Detailed Synthesis

### Example 1

One embodiment of the present disclosure provides the following synthesis of Compound I according to the route shown in Scheme 2:

### Step a) Formation of Compound 2

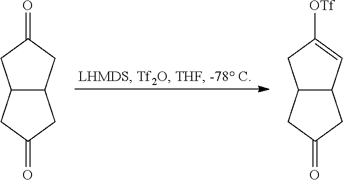 [SMILES: O=C1CC2CC(=O)CC2C1 ; O=C1CC2C=C(OS(=O)(=O)C(F)(F)F)CC2C1]

Tetrahydropentalene-2,5(1H,3H)-dione—Compound 1 is dissolved in THF and cooled to −78° C. To the solution is added lithium hexamethyldisilazane, followed by trifluoromethanesulfonic anhydride. The mixture is stirred at −78° C. until the reaction is complete. The mixture is quenched with water. The process temperature is raised to approximately 0° C. and then concentrated. The concentrated solution is diluted with ethyl acetate and the diluted solution is concentrated. The concentrated solution is washed with aqueous sodium chloride. The organic layer is concentrated. The crude product is taken up in heptane and purified by silica gel chromatography.

### Step b) Formation of Compound 3

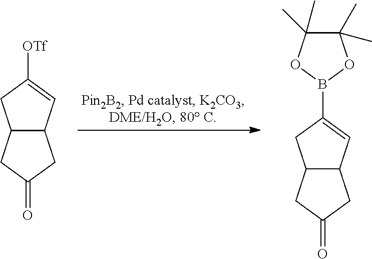

Compound 2 is dissolved in a mixture of dimethoxyethane and water. To the vinyl-triflate solution is added potassium carbonate followed by a palladium catalyst. The process temperature is increased to approximately 80° C. and stirred at that temperature until the reaction is complete. The process temperature is decreased to approximately 40° C. and the mixture is concentrated. To the mixture is added 2-methyltetrahydrofuran and water. The layers are separated, the organic layer is diluted with heptane, and the solution is filtered over silica gel. The filtrate is concentrated, and the remaining crude product is diluted with ethyl acetate, stirred at approximately 0° C. until the pure product precipitates from solution. The desired product is collected by filtration.

### Step c) Formation of Compound 3.1

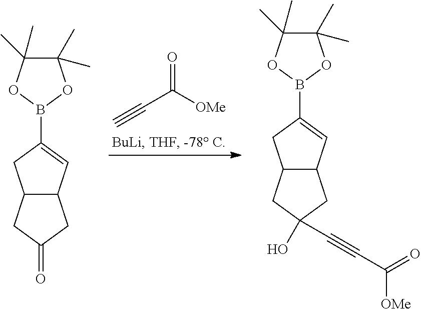

Methyl propiolate is dissolved in THF and cooled to −78° C. To the cooled alkyne solution is added Compound 3 in THF. The reaction mixture is stirred at −78° C. until complete. The mixture is quenched with aqueous ammonium chloride and the mixture is extracted with ethyl acetate. The organic layer is concentrated to dryness and the crude product is purified by silica gel chromatography. The crude product is purified by silica gel chromatography.

### Step d) Formation of Compound 3.2

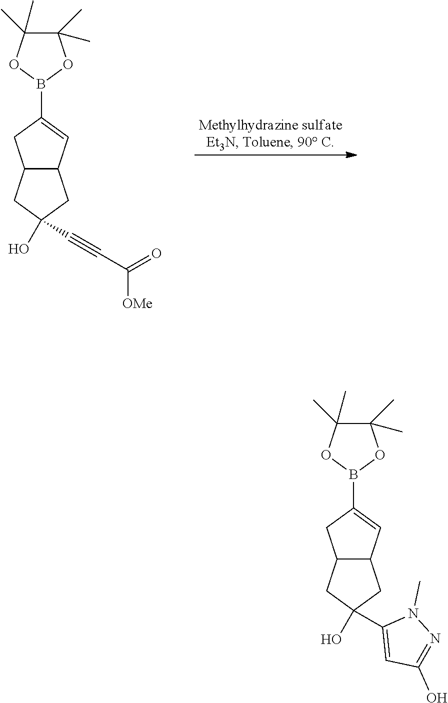 [SMILES: COC(=O)C#C[C@@]1(O)CC2C=C(B3OC(C)(C)C(C)(C)O3)CC2C1 ; Cn1nc(O)cc1C1(O)CC2C=C(B3OC(C)(C)C(C)(C)O3)CC2C1]

Methylhydrazine sulfate is suspended in toluene. To the suspension is added triethylamine and the mixture is stirred for a short period. Compound 3.1 is added. The process temperature is raised to approximately 90° C. and the reaction is stirred at that temperature until completion. The reaction mixture is cooled to ambient and quenched with water. The crude product is collected by filtration. The crude product is then taken up in isopropanol and stirred at ambient. The purified product is collected by filtration.

### Step e) Formation of Compound 3.3

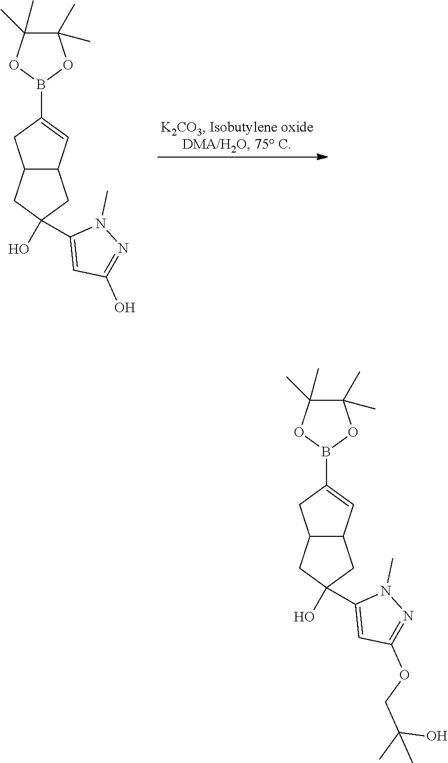 [SMILES: Cn1nc(O)cc1C1(O)CC2C=C(B3OC(C)(C)C(C)(C)O3)CC2C1 ; Cn1nc(OCC(C)(C)O)cc1C1(O)CC2C=C(B3OC(C)(C)C(C)(C)O3)CC2C1]

Compound 3.2 is dissolved in a mixture of dimethylacetamide/water. To the solution is added potassium carbonate, followed by isobutylene oxide. The process temperature is raised to 75° C. and the reaction is stirred at that temperature until it reaches completion. The reaction mixture is filtered. The filtrate is diluted with ethyl acetate and the solution is concentrated to lesser volume. The dilution/concentration sequence is repeated, and the remaining solution is used as is in the next step. Alternatively, the crude product can be purified by chromatography over silica gel.

### Step f) Formation of Compound 3.4

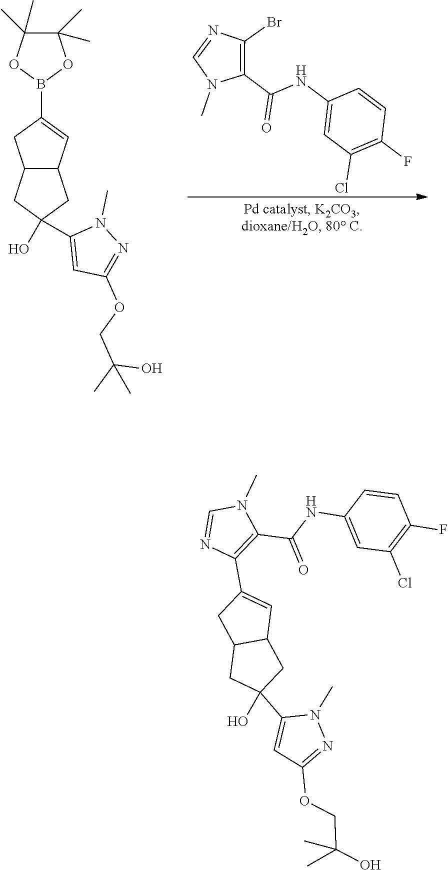 [SMILES: Cn1nc(OCC(C)(C)O)cc1C1(O)CC2C=C(B3OC(C)(C)C(C)(C)O3)CC2C1 ; Cn1cnc(Br)c1C(=O)Nc1ccc(F)c(Cl)c1 ; Cn1cnc(C2=CC3CC(O)(c4cc(OCC(C)(C)O)nn4C)CC3C2)c1C(=O)Nc1ccc(F)c(Cl)c1]

Compound 3.3 solution is diluted with a mixture of dioxane/water. To the vinyl-boronate solution is added potassium carbonate, followed by a palladium catalyst. The process temperature is raised to approximately 80° C. and the mixture is stirred at that temperature until the reaction is complete. The mixture is cooled to ambient and then filtered. The filtrate is concentrated. The crude product is diluted with water and extracted with ethyl acetate. The organic layer is washed with aqueous brine, and then concentrated. The crude product is purified by silica gel chromatography.

### Step g) Formation of Compound I

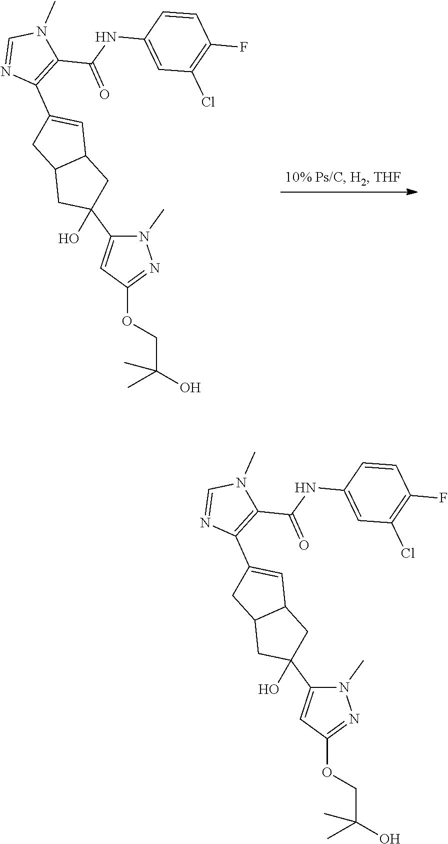 [SMILES: Cn1cnc(C2=CC3CC(O)(c4cc(OCC(C)(C)O)nn4C)CC3C2)c1C(=O)Nc1ccc(F)c(Cl)c1 ; Cn1cnc(C2=CC3CC(O)(c4cc(OCC(C)(C)O)nn4C)CC3C2)c1C(=O)Nc1ccc(F)c(Cl)c1]

Compound 3.4 is dissolved in THF. To the solution is added 10% palladium on carbon. The mixture is exposed to a hydrogen atmosphere. The mixture is stirred at ambient temperature and pressure until the reaction is complete. The mixture is filtered over celite. The filtrate is concentrated to a minimum volume and then diluted with ethyl acetate. The diluted solution is concentrated to a minimum volume and stirred at ambient temperature. The precipitated product (Compound I) is collected by filtration.

### Example 2

An embodiment of the present disclosure provides the following synthesis of Compound I based on the route shown in Scheme 2:

### Step a) Formation of Compound 2

Tetrahydropentalene-2,5(1H,3H)-dione—Compound 1 (1 g, 7.24 mmol) was dissolved in THF (15 ml, 15 Vol) and cooled to −78° C. To the solution was added lithium hexamethyldisilazane (1 M THF, 6.15 mL, 6.15 mmol, 0.85 Eq), followed by trifluoromethanesulfonic anhydride (1.35 mL, 7.96 mmol, 1.1 Eq). The mixture was stirred at −78° C. until the reaction was complete. The mixture was quenched with water (2 ml, 2 Vol). The process temperature was raised to approximately 0° C. and stirred for 30 min. The reaction mixture was poured into a mixture of ethyl acetate (15 ml, 15 Vol) and sat. NaHCO₃ (15 ml, 15 Vol). The layers were separated and the organic layer washed with brine (20 ml, 20 Vol). The organic layer was dried over Na₂SO₄, filtered, and evaporated under reduced pressure to obtain a gum. The crude product was purified via silica gel chromatography (40 g cartridge) using a gradient of cyclohexane/ethyl acetate (I/O to 1/1 over 12 CV). 5-oxo-1,3a,4,5,6,6a-hexahydropentalen-2-yl trifluoromethanesulfonate—Compound 2 was obtained as a light yellow oil (857 mg, 52% yield). ¹H NMR (400 MHz, MeOD, ppm) δ 5.72 (q, J=2.1 Hz, 1H), 3.58-3.49 (m, 1H), 3.18-3.08 (m, 1H), 3.04 (ddt, J=16/8.1/2.6 Hz, 1H), 2.67-2.48 (m, 2H), 2.47-2.39 (m, 1H), 2.30-2.21 (m, 1H), 2.11 (dd, J=19/7.2 Hz, 1H).

### Step b) Formation of Compound 3

Compound 2 (750 mg, 2.78 mmol, 1 Eq) was dissolved in a mixture of dimethoxyethane (7.5 mL, 10 Vol) and bis(pinacolato)diboron (773 mg, 3.04 mmol, 1.1 Eq) at room temperature. To the Compound 2 solution was added potassium carbonate (840 mg, 8.41 mmol, 3 Eq) followed by Pd(dppf)Cl₂ (8.2 mg, 0.011 mmol, 0.004 Eq). The process temperature was increased to approximately 80° C. and stirred at that temperature until the reaction was complete. The process temperature was decreased to room temperature and the mixture was filtered over Celite™. The filtrate was concentrated under reduced pressure to ˜1 Vol, n-heptane (1.5 mL, 2 Vol) was added, and the mixture concentrated to a minimum volume under reduced pressure. The n-heptane addition/concentration was repeated, then the crude product was dissolved in n-heptane (750 mL, 1 Vol) and purified via silica gel chromatography using a gradient of n-heptane/ethyl acetate (I/O to 10/1 over 8 CV). The fractions containing pure product were combined, concentrated to a minimum volume under reduced pressure, and precipitated by stirring in n-heptane (100 mL, 0.2 Vol) at 0-10° C. for 1-2 hours. The purified product was isolated via filtration to give 5-(4,4,5,5-tetramethyl-1,3,2-dioxaborolan-2-yl)-3,3a,4,6a-tetrahydropentalen-2(1H)-one—Compound 3 as an off-white solid (581 mg, 84% yield). ¹H NMR (400 MHz, DMSO) δ 6.27 (q, J=2.1 Hz, 1H), 3.45-3.37 (m, 1H), 2.92 (dqd, J=9.8, 7.5, 2.2 Hz, 1H), 2.64 (ddt, J=16.4, 5.1, 2.7 Hz, 1H), 2.48-2.39 (m, 2H), 2.27-2.10 (m, 2H), 1.81 (ddd, J=19.0, 6.7, 1.9 Hz, 1H), 1.20 (s, 12H).

### Step c) Formation of Compound 3.1

Methyl propiolate (281 μL, 4 Eq, 3.22 mmol) was dissolved in THF (3 ml, 15 Vol) and cooled to −78° C. n-BuLi (2.5 M in hexanes, 1.29 mL, 4 Eq, 3.22 mmol) was added dropwise to the solution and the reaction mixture stirred for 1 hour. To the cooled alkyne solution was added Compound 3 (200 mg, 1 Eq, 806 μmol) in THF (1 ml, 5 Vol). The reaction mixture was stirred at −78° C. until complete. The mixture was quenched with aqueous ammonium chloride (0.6 ml, 3 Vol) and the mixture was extracted with ethyl acetate (2×2 ml, 10 Vol). The organic layer was concentrated to dryness under reduced pressure and the crude product was purified by silica gel chromatography (0-30% ethyl acetate in cyclohexane over 12 CVs, 20 g) to give methyl 3-(2-hydroxy-5-(4,4,5,5-tetramethyl-1,3,2-dioxaborolan-2-yl)-1,2,3,3a,4,6a-hexahydropentalen-2-yl) propiolate—Compound 3.1 (182 mg, 68% yield) as a light yellow oil. ¹H NMR (400 MHz, MeOD) δ ppm: 6.33 (q, J=2.2 Hz, 1H), 3.74 (s, 3H), 3.38-3.31 (m, 1H), 2.93-2.83 (m, 1H), 2.68 (dtt, J=16.8, 9.3, 2.3 Hz, 1H), 2.38-2.24 (m, 3H), 1.77-1.63 (m, 2H), 1.25 (s, 12H).

### Step d) Formation of Compound 3.2

Methylhydrazine sulfate (775 mg, 3 Eq, 5.37 mmol) was suspended in toluene (6 mL, 10 Vol). To the suspension was added triethylamine (749 μL, 3 Eq, 5.37 mmol) and the mixture was stirred for 30-60 minutes at 20-30° C. Compound 3.1 (595 mg, 1 Eq, 1.79 mmol) was added, the process temperature was raised to approximately 90° C., and the reaction was stirred at that temperature until completion. The reaction mixture was cooled to ambient temperature and quenched with water (6 mL, 10 Vol). The crude product was collected by filtration. The crude product was then taken up in isopropanol (1.2 mL, 2 Vol) and stirred at ambient for 10-12 hours. The purified product was collected by filtration to obtain give 5-(2-hydroxy-5-(4,4,5,5-tetramethyl-1,3,2-dioxaborolan-2-yl)-1,2,3,3a,4,6a-hexahydropentalen-2-yl)-1-methyl-1H-pyrazol-3-ol—Compound 3.2 (299 mg, 48% yield) as an off-white solid. ¹H NMR (400 MHz, DMSO) δ 9.32 (s, 1H), 6.29 (d, J=2.2 Hz, 1H), 5.31 (s, 1H), 5.11 (s, 1H), 3.65 (s, 3H), 3.14 (br s, 1H), 2.60-2.52 (m, 2H), 2.34-2.17 (m, 3H), 1.67 (ddd, J=24.7, 13.0, 6.8 Hz, 2H), 1.20 (s, 12H).

### Step e) Formation of Compound 3.3

Compound 3.2 (500 mg, 1 Eq, 1.44 mmol) was dissolved in a mixture of dimethylacetamide/water (10:1 ratio, 5.5 mL, 11 Vol). To the solution was added potassium carbonate (499 mg, 2.5 Eq, 3.61 mmol), followed by isobutylene oxide (521 mg, 5 Eq, 7.22 mmol). The process temperature was raised to 75° C. and the reaction was stirred at that temperature until completion. The reaction mixture was filtered, the filtrate was diluted with ethyl acetate (2.5 mL, 5 Vol), and the solution was concentrated to ˜11 Vol under reduced pressure. The dilution/concentration sequence was repeated, and the resultant Compound 3.3 solution was used as is in the next step (˜0.26 M concentration).

### Step f) Formation of Compound 3.4

Compound 3.3 solution from the previous step (˜5.5 ml, 1 Eq, 1.44 mmol) was diluted with a mixture of dioxane/water (5:1 ratio, 6 mL, 12 Vol). To the solution was added 4-bromo-N-(4-chloro-3-fluorophenyl)-1-methyl-1H imidazole-5-carboxamide—Compound 3b (528 mg, 1.1 Eq, 1.59 mmol), and potassium carbonate (499 mg, 2.5 Eq, 3.61 mmol), followed by the palladium catalyst (cataCXium® A Pd G3, 26 mg, 0.025 Eq, 0.036 mmol). The process temperature was raised to approximately 80° C. and the mixture was stirred at that temperature until the reaction was complete. The mixture was cooled to ambient and then filtered. The filtrate was concentrated to ˜13 Vol under reduced pressure. The crude product was diluted with water (5 mL, 10 Vol) and extracted with ethyl acetate (3×3 mL/6 Vol). The organic layer was washed with aqueous brine (3×5 mL/10 Vol), and then concentrated under reduced pressure to ˜1 Vol. The crude product was purified by silica gel chromatography to give N-(4-chloro-3-fluorophenyl)-4-(5-hydroxy-5-(3-(2-hydroxy-2-methylpropoxy)-1-methyl-1H-pyrazol-5-yl)-1,3a,4,5,6,6a-hexahydropentalen-2-yl)-1-methyl-1H-imidazole-5-carboxamide—Compound 3.4 as a white solid (385 mg, 49% yield over two steps). ¹H NMR (400 MHz, DMSO) δ 10.56 (s, 1H), 8.01 (dd, J=6.8, 2.5 Hz, 1H), 7.70 (s, 1H), 7.62-7.55 (m, 1H), 7.42 (t, J=9.1 Hz, 1H), 6.01 (d, J=2.2 Hz, 1H), 5.56 (s, 1H), 5.26 (s, 1H), 4.52 (s, 1H), 3.73 (s, 2H), 3.69 (s, 3H), 3.65 (s, 3H), 3.19 (d, J=8.2 Hz, 1H), 2.85 (dd, J=16.1, 9.2 Hz, 1H), 2.62 (t, J=8.5 Hz, 1H), 2.44 (br s, 1H), 2.37-2.24 (m, 2H), 1.72 (td, J=13.0, 7.5 Hz, 2H), 1.13 (s, 6H).

### Step g) Formation of Compound I

Compound 3.4 (250 mg, 0.46 mmol, 1 Eq) was dissolved in THF (5 mL, 20 Vol). To this solution was added 10% palladium on carbon (0.1 w/w, 25 mg, 0.01 Eq). The mixture was exposed to a hydrogen atmosphere. The mixture was stirred at ambient temperature and pressure until the reaction was complete. The mixture was filtered over Celite™. The filtrate was concentrated to a minimum volume under reduced pressure and then diluted with ethyl acetate (1.25 mL, 5V). The diluted solution was concentrated to ˜2 Vol under reduced pressure and stirred at ambient temperature for 2-4 hours. The precipitated product was collected by filtration to give Compound I as an off-white solid (213 mg, 85% yield). ¹H NMR (400 MHz, DMSO-d₆): δ 10.22 (s, 1H), 7.96 (dd, J=6.8 Hz, 2.4 Hz, 1H), 7.65 (s, 1H), 7.59-7.52 (m, 1H), 7.40 (t, J=9.6 Hz, 1H), 5.52 (s, 1H), 5.23 (s, 1H), 4.53 (s, 1H), 3.75-3.70 (m, 5H), 3.67 (s, 3H), 3.26-3.20 (m, 1H), 2.50-2.44 (m, 2H), 2.20-2.06 (m, 4H), 1.90-1.80 (m, 4H), 1.13 (s, 6H) ppm. MS calcd. for C₂₇H₃₃ClFN₅O₄: 545.2; Found: 546.3 [M+1]⁺.

### Example 3

The synthesis of Compound I was further carried out according to the route shown in Scheme 3. After isolation and purification of Compound I, the stereochemistry of Compound I was determined by single crystal X-Ray diffraction as Compound I(a):

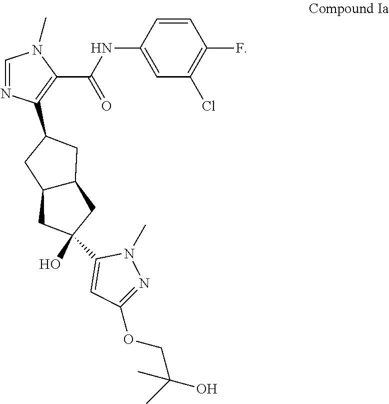 [SMILES: Cn1cnc([C@@H]2C[C@@H]3C[C@@](O)(c4cc(OCC(C)(C)O)nn4C)C[C@@H]3C2)c1C(=O)Nc1ccc(F)c(Cl)c1]

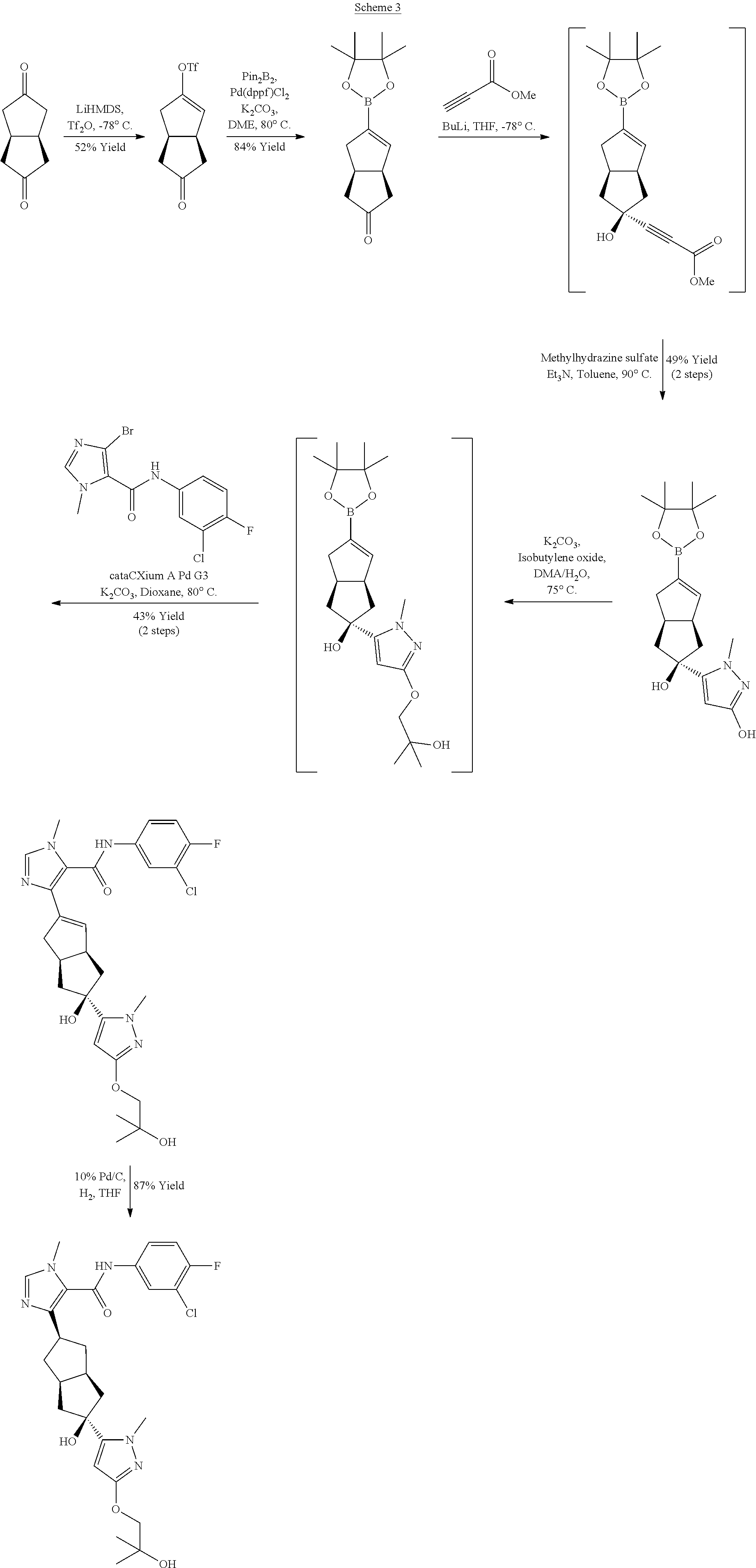

The following modifications were made to the process described in Example 2.

### Example 3A—Telescoped Steps c) and d) to Produce Compound 3.2

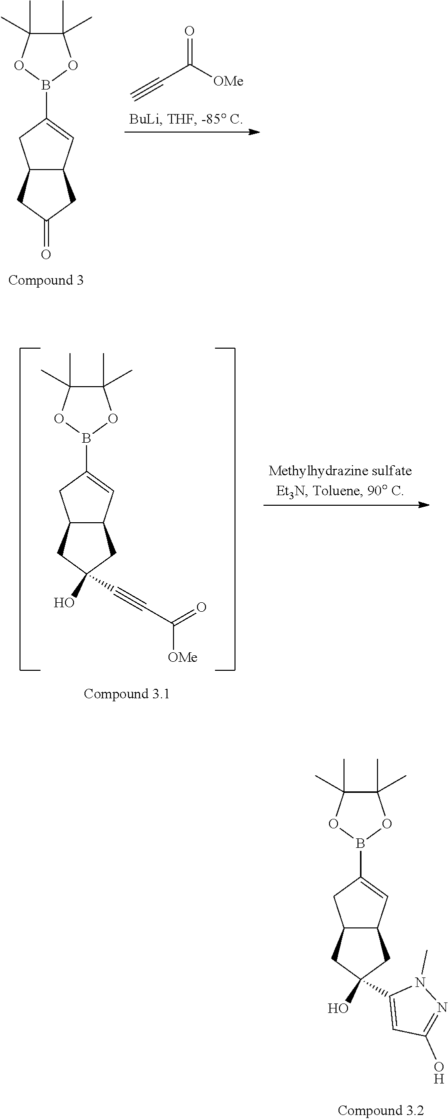

Methyl propiolate (13.55 g, 4 Eq, 0.161 mol) was dissolved in THF (400 ml, 40 Vol) and combined with n-BuLi (1.6 M in THF, 106 mL, 4.2 Eq, 0.169 mol) in a stainless steel flow reactor at −85° C. (13 mLΦ6, Rt=1.0 min). The output of the flow reactor was continuously added to a standard batch reactor containing a solution of Compound 3 (10.0 g, 1 Eq, 0.040 mol) in THF (100 ml, 10 Vol) at −85° C. After addition of the methyl propiolate/n-BuLi solution was complete the reaction mixture was stirred for two hours at −85° C. The reaction mixture was warmed to −60° C. and the pH adjusted to 6-8 via addition of a solution of acetic acid in THF (1:3 v/v acetic acid/THF). The quenched reaction mixture was warmed to ambient temperature, washed with water (50 ml, 5 Vol), the phases were separated, and the organic phase was concentrated under reduced pressure to 1-2 Vol. Toluene was added (50 mL, 5 Vol), the mixture was concentrated to 1-2 Vol under reduced pressure, and the toluene addition/concentration steps were repeated. Toluene was added (150 ml, 15 Vol), the amount of Compound 3.1 assayed via HPLC (87% assay yield), and the crude product in toluene (˜0.23 M) was used directly in the next step.

Methylhydrazine sulfate (15.16 g, 3 Eq, 0.105 mol) and triethylamine (14.66 mL, 3 Eq, 0.105 mol) were added to the solution of Compound 3.1 in toluene (150 mL, 1 Eq, 0.035 mol). The process temperature was raised to approximately 90° C. and the reaction was stirred at that temperature until completion. The reaction mixture was cooled to ambient temperature and quenched with water (116 mL, 10 Vol). The crude product was collected by filtration. The crude product was then taken up in isopropanol (23.3 mL, 2 Vol) and stirred at ambient temperature for 10-12 hours. The purified product was collected by filtration to obtain Compound 3.2 (6.84 g, 49% yield over two steps) as an off-white solid. ¹H NMR (400 MHz, DMSO) δ 9.32 (s, 1H), 6.29 (d, J=2.2 Hz, 1H), 5.31 (s, 1H), 5.11 (s, 1H), 3.65 (s, 3H), 3.14 (br s, 1H), 2.60-2.52 (m, 2H), 2.34-2.17 (m, 3H), 1.67 (ddd, J=24.7, 13.0, 6.8 Hz, 2H), 1.20 (s, 12H).

### Example 3B—Telescoped Steps e) and f) with Compound 3.4 Crystallization Procedure

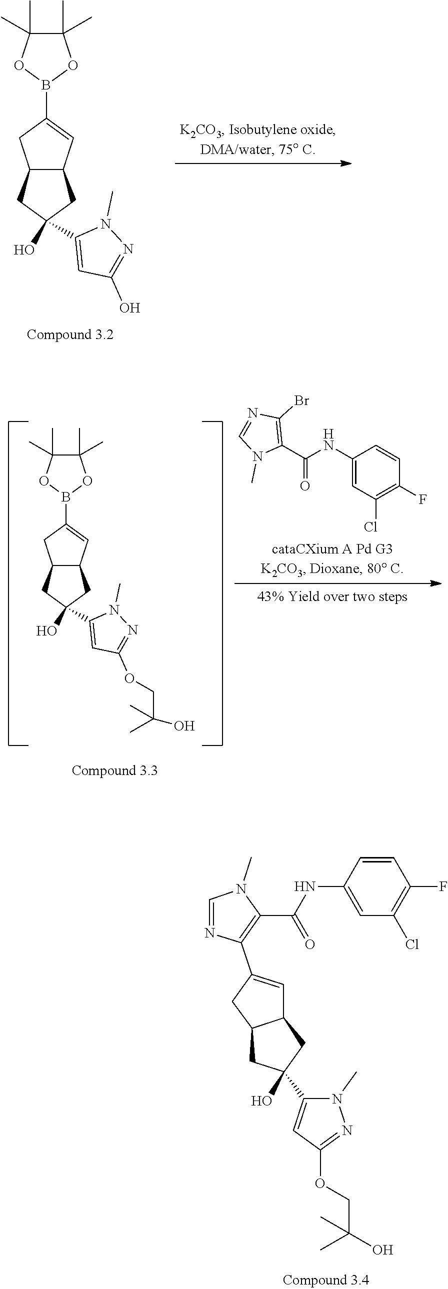

Compound 3.2 (10.0 g, 1 Eq, 28.8 mmol) was dissolved in a mixture of dimethylacetamide/water (10:1 ratio, 110 mL, 11 Vol). To the solution was added potassium carbonate (9.98 g, 2.5 Eq, 72 mmol), followed by isobutylene oxide (10.4 g, 5 Eq, 144 mmol). The process temperature was raised to 75° C. and the reaction was stirred at that temperature until it reached completion. The reaction mixture was filtered, the filtrate was diluted with ethyl acetate (50 mL, 5 Vol), and the solution was concentrated to ˜11 Vol under reduced pressure. The dilution/concentration sequence was repeated, and the obtained solution (˜0.26 M concentration) of Compound 3.3 was used as is in the next step.

The Compound 3.3 solution from the previous step (˜110 ml, 1 Eq, 28.8 mmol) was diluted with a mixture of dioxane/water (5:1 ratio, 120 mL, 12 Vol). To this solution was added 4-bromo-N-(4-chloro-3-fluorophenyl)-1-methyl-1H imidazole-5-carboxamide (10.56 g, 1.1 Eq, 31.8 mmol) and potassium carbonate (9.98 g, 2.5 Eq, 72 mmol), followed by the palladium catalyst (cataCXium® A Pd G3, 516 mg, 0.025 Eq, 0.72 mmol). The process temperature was raised to approximately 80° C. and the mixture was stirred at that temperature until the reaction was complete. The mixture was cooled to ambient temperature and then filtered. The filtrate was concentrated to ˜13 Vol under reduced pressure. The crude product was diluted with water (100 mL, 10 Vol). and extracted with ethyl acetate (3×60 mL/6 Vol). The combined organic layers were washed with aqueous brine (3×60 mL/6 Vol), and then concentrated under reduced pressure to ˜1 Vol. THF (70 mL, 7 Vol) was added to the crude product, the temperature raised to 55° C., and the mixture stirred until complete dissolution occurs. The resulting pale-yellow solution was filtered through a 0.45 μM membrane and concentrated under reduced pressure at 55° C. to ˜3 Vol to give a cloudy solution. EtOH (90 mL, 9 Vol) was added to the mixture over 30 minutes with stirring to give a suspension and the process temperature reduced to 20° C. at a rate of 5° C. per hour. The mixture was stirred at 20° C. for 12 hours and isolated via filtration. The filter cake was washed with EtOH (2×20 mL/2 Vol) and then dried at 50° C. in vacuum oven for 24 hours. Compound 3.4 was obtained as a white to off-white solid (6.74 g, 43% yield over two steps). ¹H NMR (400 MHz, DMSO) δ 10.56 (s, 1H), 8.01 (dd, J=6.8, 2.5 Hz, 1H), 7.70 (s, 1H), 7.62-7.55 (m, 1H), 7.42 (t, J=9.1 Hz, 1H), 6.01 (d, J=2.2 Hz, 1H), 5.56 (s, 1H), 5.26 (s, 1H), 4.52 (s, 1H), 3.73 (s, 2H), 3.69 (s, 3H), 3.65 (s, 3H), 3.19 (d, J=8.2 Hz, 1H), 2.85 (dd, J=16.1, 9.2 Hz, 1H), 2.62 (t, J=8.5 Hz, 1H), 2.44 (br s, 1H), 2.37-2.24 (m, 2H), 1.72 (td, J=13.0, 7.5 Hz, 2H), 1.13 (s, 6H).

### Example 3C—Purification and Recrystallisation of Compound I

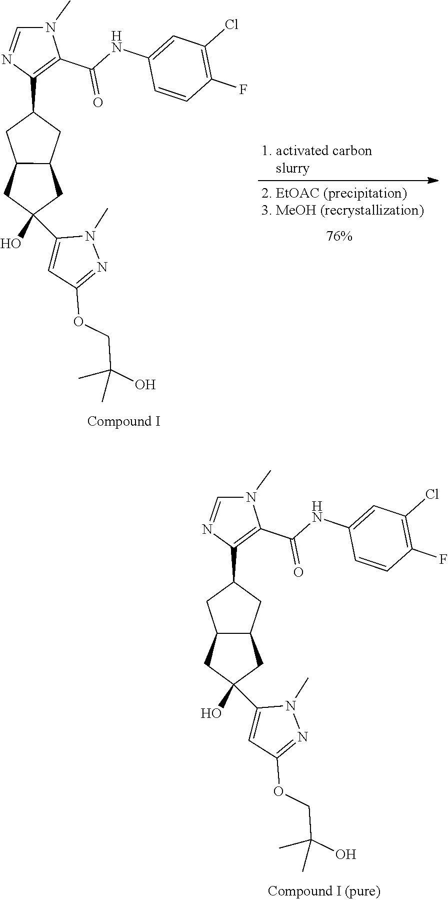 [SMILES: Cn1cnc([C@@H]2C[C@@H]3C[C@@](O)(c4cc(OCC(C)(C)O)nn4C)C[C@@H]3C2)c1C(=O)Nc1ccc(F)c(Cl)c1 ; Cn1cnc([C@@H]2C[C@@H]3C[C@@](O)(c4cc(OCC(C)(C)O)nn4C)C[C@@H]3C2)c1C(=O)Nc1ccc(F)c(Cl)c1]

After completion of the hydrogenation reaction and filtration over Celite, the hydrogenation liquor containing Compound I in THF (˜0.03 M) is combined with activated carbon (ZX-777, 0.5 wt % based on Compound 3.4 input). The mixture is stirred at 45-55° C. for 12-18 hours and then filtered over Celite. The filtrate is concentrated to 2-5 Vol under reduced pressure and EtOAc (8 Vol) is added. The mixture is concentrated to 2-3 Vol under reduced pressure and the EtOAc addition (8 Vol)/concentration steps repeated twice more. After the final concentration step, EtOAc (3 Vol) is added and the mixture stirred at ambient temperature for 1-2 hours. The temperature is reduced to 0-5° C. at a rate of 5-10° C. per hour and stirred at 0-5° C. for 10-24 hours. The precipitated product is collected by filtration to give crude Compound I. The crude Compound I is combined with MeOH (6 Vol based on weight of crude), heated to 50-60° C., and stirred until a homogenous solution is obtained. The solution is filtered and the temperature reduced to 40-50° C. Seed crystal (0.01 wt % based on weight of crude) is charged, stirred for 2-3 hours to give a cloudy solution, and the mixture concentrated to 2-4 Vol under reduced pressure at 40-50° C. The temperature is reduced to 0-10° C. at a rate of 5-10° C. per hour and stirred at 0-10° C. for 12-24 hours. The solid is collected via filtration to give pure Compound I as a white solid (87% yield for purification steps).

### Example 4—Alternative Route to Compound 3.2

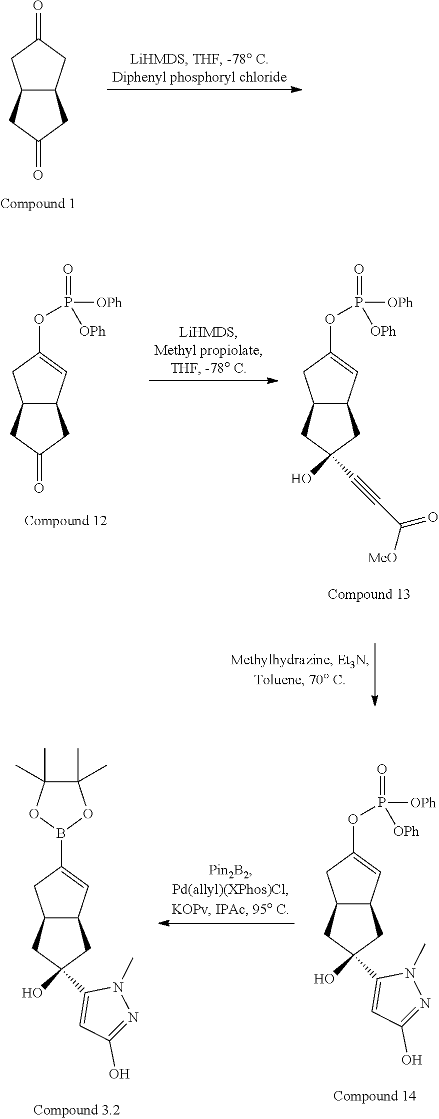

Step a)—Formation of Compound 12

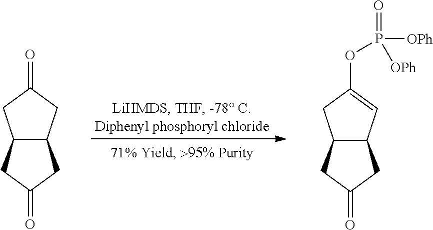 [SMILES: O=C1C[C@H]2CC(=O)C[C@H]2C1 ; O=C1C[C@H]2CC(OP(=O)(Oc3ccccc3)Oc3ccccc3)=C[C@H]2C1]

A solution of tetrahydropentalene-2,5(1H,3H)-dione—Compound 1 (10.0 g, 1 Eq, 72.4 mmol) in dry THF (150 mL, 15 vol) under N₂ was cooled to −78° C. LiHMDS (61.5 mL, 1 molar in THF, 0.85 Eq, 61.5 mmol) was added dropwise over a period of 25 mins and stirred for 20 mins. Diphenyl phosphoryl chloride (16.5 mL, 1.1 Eq, 79.6 mmol) was added dropwise over 15 mins to the solution and the reaction mixture was stirred at −78° C. for 4 hours. 20 mL of water was added and the reaction mixture was stirred for 5 mins. The dry ice bath was removed and the reaction mixture was stirred for 25 mins. The reaction mixture was diluted with ethyl acetate, washed with a saturated NaHCO₃ solution, brine, dried over Na₂SO₄ and concentrated under vacuum. The residue was purified by flash column chromatography (0-50% ethyl acetate in cyclohexane over 10 CVs, 330 g) to afford 5-oxo-1,3a,4,5,6,6a-hexahydropentalen-2-yl diphenyl phosphate—Compound 12 (16.1 g, 43.5 mmol, 71%) as a light yellow oil. ¹H NMR (400 MHz, DMSO) δ 7.52-7.40 (m, 4H), 7.35-7.20 (m, 6H), 5.36 (p, J=1.9 Hz, 1H), 3.39 (dtd, J=9.7, 5.3, 2.8 Hz, 1H), 3.01-2.92 (m, 1H), 2.85 (ddq, J=16.1, 8.1, 2.4 Hz, 1H), 2.59-2.51 (m, 1H), 2.46-2.36 (m, 1H), 2.23 (dt, J=15.9, 2.1 Hz, 1H), 2.12 (dt, J=18.7, 2.4 Hz, 1H), 1.93 (ddd, J=18.9, 6.8, 1.9 Hz, 1H).

Step b)—Formation of Compound 13

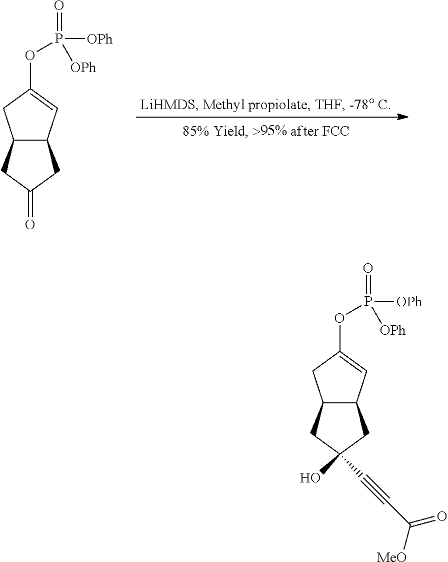 [SMILES: O=C1C[C@H]2CC(OP(=O)(Oc3ccccc3)Oc3ccccc3)=C[C@H]2C1 ; COC(=O)C#C[C@]1(O)C[C@H]2CC(OP(=O)(Oc3ccccc3)Oc3ccccc3)=C[C@H]2C1]

A solution of methyl propiolate (3.43 mL, 2.5 Eq, 39.8 mmol) in dry THF (89 ml, 15 vol) was cooled to −78° C. LiHMDS (31.9 mL, 1 molar in THF, 2 Eq, 31.9 mmol) was added dropwise and the reaction mixture was stirred for 1 hour. A solution of Compound 12 (5.90 g, 1 Eq, 15.9 mmol) in dry THF (30 ml, 5 vol) was added dropwise and the reaction was stirred for 90 minutes. The reaction mixture was quenched with 4 mL of acetic acid and was allowed to reach room temperature. The reaction mixture was diluted with ethyl acetate, washed with a saturated NaHCO₃ solution, brine and dried over Na₂SO₄. The residue was purified by flash column chromatography (0-30% ethyl acetate in cyclohexane, 120 g) to afford methyl 3-(5-((diphenoxyphosphoryl)oxy)-2-hydroxy-1,2,3,3a,4,6a-hexahydropentalen-2-yl)propiolate—Compound 13 (6.13 g, 13.5 mmol, 85%) as a yellow oil, which solidified overtime. ¹H NMR (300 MHz, DMSO) δ 7.51-7.38 (m, 4H), 7.35-7.17 (m, 6H), 5.90 (s, 1H), 5.35 (p, J=1.9 Hz, 1H), 3.71 (s, 3H), 3.19 (s, 1H), 2.86-2.61 (m, 2H), 2.38-2.26 (m, 1H), 2.17 (dt, J=13.0, 8.4 Hz, 2H), 1.75 (ddd, J=15.8, 12.9, 5.6 Hz, 2H).

Step c)—Formation of Compound 14

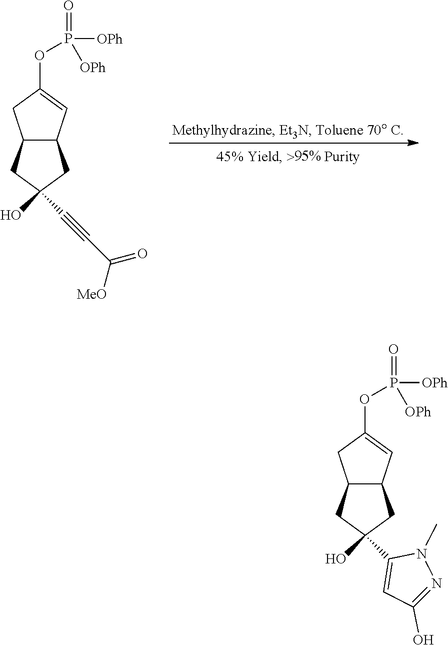 [SMILES: COC(=O)C#C[C@]1(O)C[C@H]2CC(OP(=O)(Oc3ccccc3)Oc3ccccc3)=C[C@H]2C1 ; Cn1nc(O)cc1[C@]1(O)C[C@H]2CC(OP(=O)(Oc3ccccc3)Oc3ccccc3)=C[C@H]2C1]

To a solution of methyl hydrazine sulfate (5.84 g, 3 Eq, 40.5 mmol) in dry toluene (73 mL, 12 vol) was added triethylamine (5.64 mL, 3 Eq, 40.5 mmol) at room temperature. The reaction mixture was stirred for 1 hour under N₂. A solution of Compound 13 (6.13 g, 1 Eq, 13.5 mmol) in dry toluene (30 mL, 5 vol) was added over 15 mins and the reaction mixture was stirred at 70° C. for 72 hours. The reaction mixture was heated at 80° C. and left stirring for 16 hours. The reaction mixture was allowed to reach room temperature and was quenched with acetone (1.98 mL, 2 Eq, 27.0 mmol) and left stirring for 30 mins. The reaction mixture was diluted with DCM (300 mL) and water (200 mL) was added. The reaction mixture was filtered to afford 5-hydroxy-5-(3-hydroxy-1-methyl-1H-pyrazol-5-yl)-1,3a,4,5,6,6a-hexahydropentalen-2-yl diphenyl phosphate—Compound 14 (2.84 g, 6.06 mmol, 45%) as a white solid. ¹H NMR (400 MHz, DMSO) δ 9.35 (s, 1H), 7.50-7.40 (m, 4H), 7.34-7.23 (m, 6H), 5.38 (p, J=1.9 Hz, 1H), 5.31 (s, 1H), 5.27 (s, 1H), 3.68 (s, 3H), 3.11 (d, J=9.1 Hz, 1H), 2.75-2.58 (m, 2H), 2.41-2.31 (m, 1H), 2.24 (td, J=12.9, 8.7 Hz, 2H), 1.78 (ddd, J=21.3, 12.9, 6.0 Hz, 2H).

Step d)—Formation of Compound 3.2

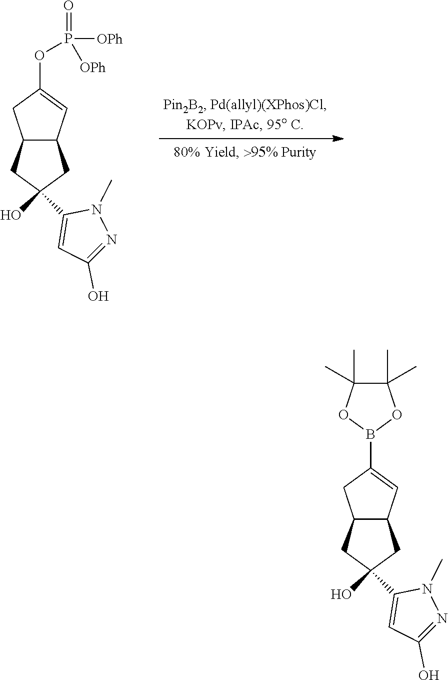

Potassium pivalate (89 mg, 2.2 Eq, 634 μmol), dicyclohexyl(2′,4′,6′-triisopropyl-[1,1′-biphenyl]-2-yl)phosphane (2.75 mg, 0.02 Eq, 5.76 μmol), Compound 14 (135 mg, 1 Eq, 288 μmol) and bis(pinacolato)diboron (88 mg, 1.2 Eq, 346 μmol) were weighed into a flask and evaporated once from toluene (5 ml). After evaporation of the toluene the rotary evaporator was also flushed with nitrogen. The residue was dissolved in dry isopropyl acetate (0.5 mL, 10 vol) and purged with N₂. Pd(XPhos)allylCl (3.80 mg, 0.02 Eq, 5.76 μmol) was added under a stream of nitrogen. The system was again evacuated and backfilled with N₂ and heated to 95° C. for 3 hours. The reaction mixture was allowed to cool to room temperature, was filtered and washed with ethyl acetate. The filtrate was washed with a saturated NaHCO₃ solution and brine, then dried over Na₂SO₄ and concentrated under vacuum. The residue was purified by flash column chromatography (0-100% ethyl acetate in cyclohexane, 12 g) to yield 5-(2-hydroxy-5-(4,4,5,5-tetramethyl-1,3,2-dioxa-borolan-2-yl)-1,2,3,3a,4,6a-hexahydropentalen-2-yl)-1-methyl-1H-pyrazol-3-ol—Compound 3.2 (80 mg, 231 μmol, 80%) as a light-yellow oil. ¹H NMR (400 MHz, DMSO) δ 9.32 (s, 1H), 6.29 (d, J=2.2 Hz, 1H), 5.31 (s, 1H), 5.11 (s, 1H), 3.65 (s, 3H), 3.14 (br s, 1H), 2.60-2.52 (m, 2H), 2.34-2.17 (m, 3H), 1.67 (ddd, J=24.7, 13.0, 6.8 Hz, 2H), 1.20 (s, 12H).

The route from Compound 1 to Compound 3.2 described in Example 4 is four steps with an overall yield of 21.7%. This compares favorably to the four step route from Compound 1 to Compound 3.2 described in Example 2, which had an overall yield of 14.3%. In particular, the desymmetrization of the diketone (Compound 1) to the vinyl diphenyl phosphate (Compound 12) proceeds in much higher yield (71%) than the corresponding conversion of the diketone to the vinyl triflate (Compound 2; 52%); and the amount of the methyl propiolate organometallic species required was reduced to 2 equivalents for the formation of Compound 13, compared to 4 equivalents used for the formation of Compound 3.1.

### Example 5—Formation of Compound 12.1

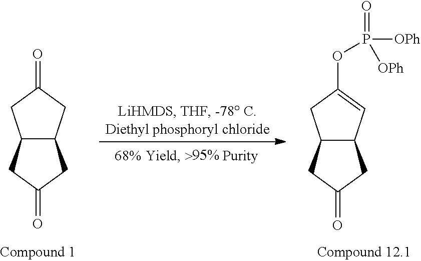 [SMILES: O=C1C[C@H]2CC(=O)C[C@H]2C1 ; O=C1C[C@H]2CC(OP(=O)(Oc3ccccc3)Oc3ccccc3)=C[C@H]2C1]

A solution of tetrahydropentalene-2,5(1H,3H)-dione—Compound 1 (500 mg, 1 Eq, 3.62 mmol) in dry THF (7.5 mL, 15 vol) under N₂ was cooled to −78° C. LiHMDS (3.08 mL, 1 molar in THF, 0.85 Eq, 3.08 mmol) was added dropwise over a period of 4 mins and stirred for 20 mins. Diethyl phosphoryl chloride (576 μL, 1.1 Eq, 3.98 mmol) was added dropwise over a period of 7 mins to the solution and the reaction mixture was stirred at −78° C. for 4 hours. 1 mL of water was added and the reaction mixture was stirred for 5 mins. The dry ice bath was removed and the reaction mixture was stirred for 25 mins. The reaction mixture was diluted with ethyl acetate and washed with a saturated NaHCO₃ solution. The aqueous layer was extracted with CH₂Cl₂. The combined organic layers were dried over Na₂SO₄ and concentrated under vacuum. The residue was purified by flash column chromatography (0-75% ethyl acetate in cyclohexane, 40 g) to yield diethyl (5-oxo-1,3a,4,5,6,6a-hexahydropentalen-2-yl) phosphate—Compound 12.1 (570 mg, 2.08 mmol, 68%) as a light yellow oil. ¹H NMR (400 MHz, DMSO) δ 5.17 (p, J=1.9 Hz, 1H), 4.08 (ddt, J=8.5, 7.8, 6.8 Hz, 4H), 3.36 (dtd, J=9.7, 5.1, 2.5 Hz, 1H), 2.99-2.88 (m, 1H), 2.81 (ddq, J=16.1, 8.1, 2.3 Hz, 1H), 2.59-2.52 (m, 1H), 2.44 (ddd, J=18.6, 9.4, 1.8 Hz, 1H), 2.21 (dt, J=16.1, 2.1 Hz, 1H), 2.10 (dt, J=18.5, 2.5 Hz, 1H), 1.95 (ddd, J=18.9, 6.5, 1.6 Hz, 1H), 1.25 (tt, J=7.1, 1.0 Hz, 6H).

### Example 6—Synthesis of Compound 3.4 from Compound 14

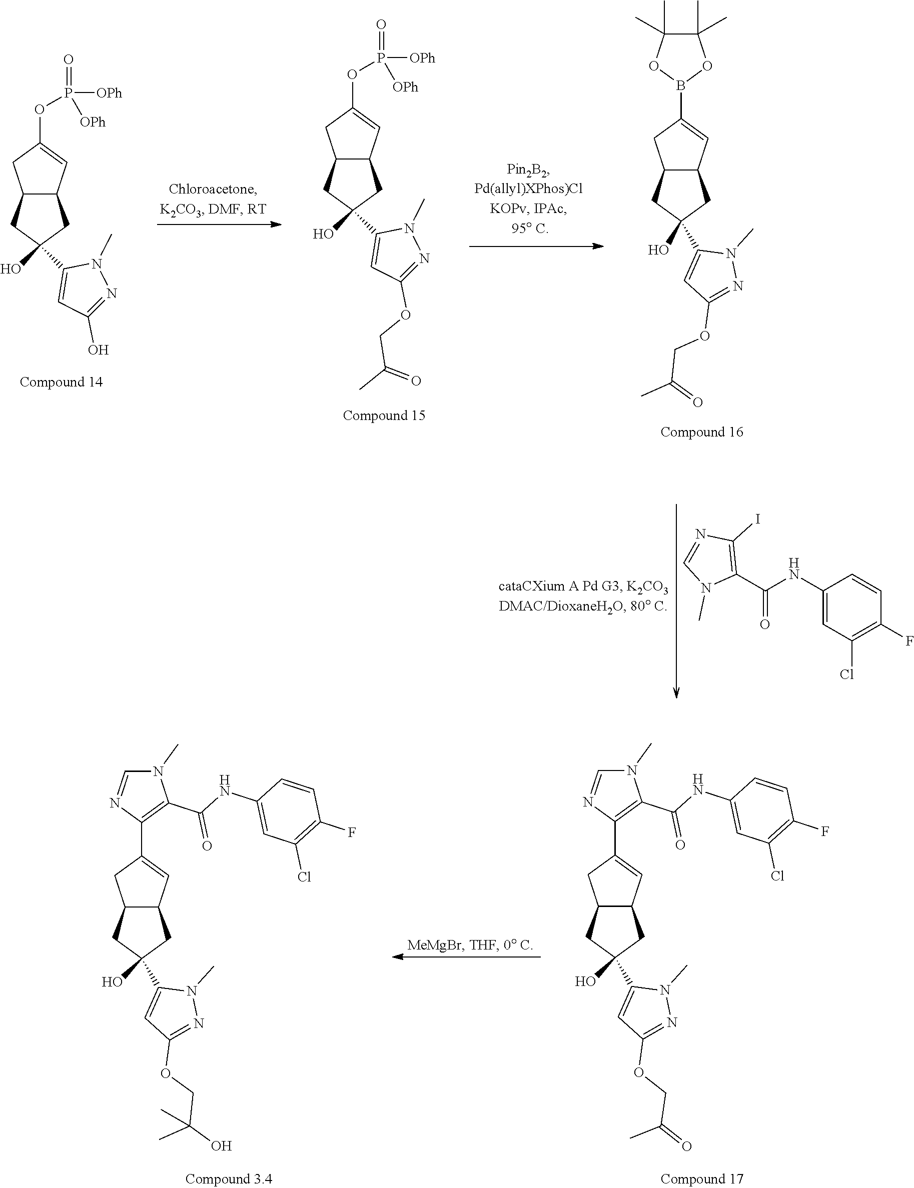

Step a)—Formation of Compound 15

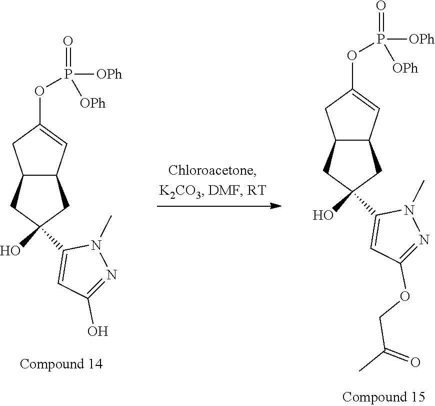 [SMILES: Cn1nc(O)cc1[C@]1(O)C[C@H]2CC(OP(=O)(Oc3ccccc3)Oc3ccccc3)=C[C@H]2C1 ; CC(=O)COc1cc([C@]2(O)C[C@H]3CC(OP(=O)(Oc4ccccc4)Oc4ccccc4)=C[C@H]3C2)n(C)n1]

Compound 14 (1.00 g, 1 Eq, 2.13 mmol), chloroacetone (341 μL, 2 Eq, 4.27 mmol), and K₂CO₃ (354 mg, 1.2 Eq, 2.56 mmol) were dissolved in DMF (10.7 mL, 11 vol) and stirred at room temperature for 16 hours. The reaction mixture was diluted with ethyl acetate (100 mL) and washed with water (3×50 mL). The organic layer was dried over Na₂SO₄ and concentrated under vacuum. The residue was purified by flash column chromatography (0-100% ethyl acetate in cyclohexane, 10 CVs, then 5 CVs at 20% MeOH in DCM, 40 g) to afford 5-hydroxy-5-(1-methyl-3-(2-oxopropoxy)-1H-pyrazol-5-yl)-1,3a,4,5,6,6a-hexahydropentalen-2-yl diphenyl phosphate—Compound 15 (1.00 g, 1.7 mmol, 78%, 87% purity) as a light yellow oil. ¹H NMR (400 MHz, DMSO) δ 7.50-7.41 (m, 4H), 7.35-7.21 (m, 6H), 5.59 (s, 1H), 5.38 (m, 2H), 4.68 (s, 2H), 3.72 (s, 3H), 3.10 (br s, 1H), 2.72-2.61 (m, 2H), 2.39-2.21 (m, 3H), 2.09 (s, 3H), 1.80 (ddd, J=19.7, 12.9, 6.1 Hz, 3H).

Step b)—Formation of Compound 16

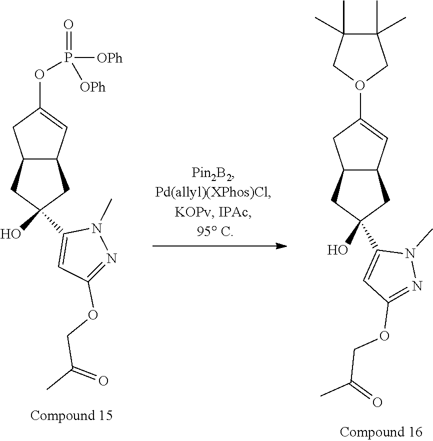

Potassium pivalate (374 mg, 2.2 Eq, 2.66 mmol), dicyclohexyl(2′,4′,6′-triisopropyl-[1,1′-biphenyl]-2-yl)phosphane (11.5 mg, 0.02 Eq, 24.2 μmol), Compound 15 (730 mg, 87% Wt, 1 Eq, 1.21 mmol) and bis(pinacolato)diboron (369 mg, 1.2 Eq, 1.45 mmol) were weighed into a flask and evaporated once from toluene (10 ml). After evaporation of the toluene, the rotary evaporator was also flushed with nitrogen. The residue was dissolved in dry isopropyl acetate (7.3 mL, 10 vol) and purged with N₂. Pd(XPhos)allylCl (16.0 mg, 0.02 Eq, 24.2 μmol) was added under a stream of nitrogen. Microwave vial was purged again with N₂ and heated to 95° C. for 2.5 hours. The reaction mixture was allowed to cool to room temperature, was filtered and washed with ethyl acetate. The filtrate was washed with a saturated NaHCO₃ solution and brine, then dried over Na₂SO₄ and concentrated under vacuum. The residue was dissolved in ethyl acetate, filtrate on a pad of silica and washed with ethyl acetate. The filtrate was concentrated under vacuum to afford 1-((5-(2-hydroxy-5-(4,4,5,5-tetramethyl-1,3,2-dioxaborolan-2-yl)-1,2,3,3a,4,6a-hexahydropenta-len-2-yl)-1-methyl-1H-pyrazol-3-yl)oxy)propan-2-one—Compound 16 (580 mg, 0.97 mmol, 80%, 67% Purity) as an oil. ¹H NMR (400 MHz, MeOD) δ 6.39 (m, 1H), 5.65 (s, 1H), 4.69 (s, 2H), 3.80 (s, 3H), 3.28-3.22 (m, 1H), 2.73-2.62 (m, 2H), 2.50-2.30 (m, 3H), 2.17 (s, 3H), 1.86-1.71 (m, 2H), 1.26 (s, 12H).

Step c)—Formation of Compound 17

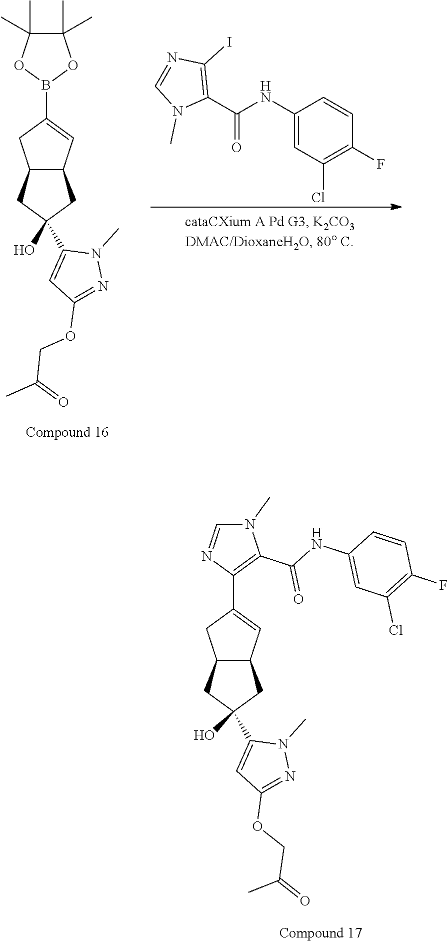

Compound 16 (290 mg, 67% Wt, 1 Eq, 483 μmol) was dissolved in a mixture of DMAc (2 mL, 10 vol), 1,4-Dioxane (2 mL, 10 vol) and water (0.8 mL, 4 vol). N-(4-chloro-3-fluorophenyl)-4-iodo-1-methyl-1H-imidazole-5-carboxamide (192 mg, 1.05 Eq, 507 μmol), potassium carbonate (167 mg, 2.5 Eq, 1.21 mmol) and cataCXium® A Pd G3 (8.79 mg, 0.025 Eq, 12.1 μmol) were added and the reaction mixture was degassed for 5 mins while sonicating. The reaction mixture was placed in a preheated heating block and left stirring at 80° C. for 40 hours. The reaction was quenched with a saturated NH₄Cl solution, extracted twice with ethyl acetate, dried over Na₂SO₄ and concentrated under vacuum. The residue was purified by flash column chromatography (0-10% MeOH in CH₂Cl₂) to afford N-(4-chloro-3-fluorophenyl)-4-(5-hydroxy-5-(1-methyl-3-(2-oxopropoxy)-1H-pyrazol-5-yl)-1,3a,4,5,6,6a-hexahydropentalen-2-yl)-1-methyl-1H-imidazole-5-carboxamide—Compound 17 (77 mg, 0.15 mmol, 30%) as a white solid. ¹H NMR (400 MHz, DMSO) δ 10.56 (s, 1H), 8.01 (m, 1H), 7.70 (s, 1H), 7.58 (ddd, J=9.1, 4.4, 2.4 Hz, 1H), 7.42 (t, J=9.1 Hz, 1H), 6.01 (d, J=2.2 Hz, 1H), 5.61 (s, 1H), 5.31 (s, 1H), 4.67 (s, 2H), 3.67 (s, 3H), 3.66 (s, 3H), 3.123-3.13 (m, 1H), 2.85 (dd, J=16.2, 9.1 Hz, 1H), 2.64-2.57 (m, 1H), 2.47-2.43 (m, 1H), 2.37-2.24 (m, 2H), 2.08 (s, 3H), 1.72 (td, J=12.9, 7.6 Hz, 2H).

Step d)—Formation of Compound 3.4

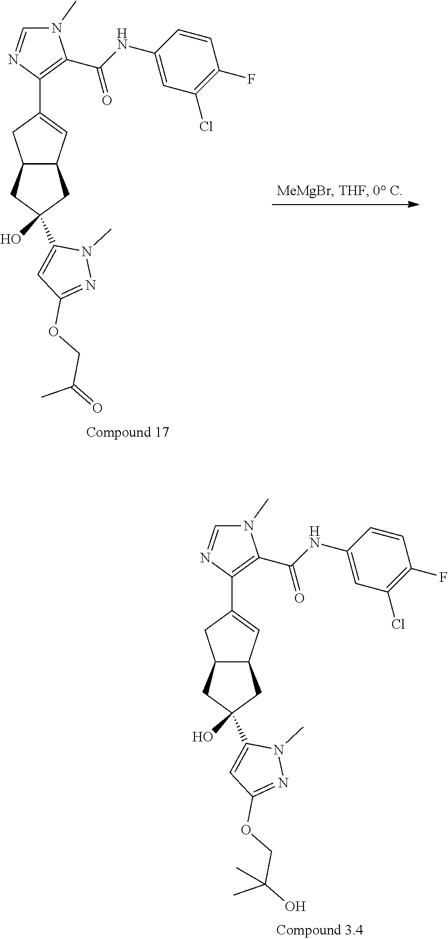 [SMILES: CC(=O)COc1cc([C@]2(O)C[C@H]3CC(c4ncn(C)c4C(=O)Nc4ccc(F)c(Cl)c4)=C[C@H]3C2)n(C)n1 ; Cn1cnc(C2=C[C@H]3C[C@](O)(c4cc(OCC(C)(C)O)nn4C)C[C@H]3C2)c1C(=O)Nc1ccc(F)c(Cl)c1]

A solution of 3 M methyl magnesium bromide in diethyl ether (212 μL, 4 Eq, 636 μmol) was added to a solution of Compound 17 (84.0 mg, 1 Eq, 159 μmol) in dry THF (2 mL) at 0° C. The reaction mixture was stirred for 1 hour, then was quenched with a saturated NH₄Cl solution, extracted twice with ethyl acetate, dried over Na₂SO₄ and concentrated under vacuum to afford N-(4-chloro-3-fluorophenyl)-4-(5-hydroxy-5-(3-(2-hydroxy-2-methylpropoxy)-1-methyl-1H-pyrazol-5-yl)-1,3a,4,5,6,6a-hexahydropentalen-2-yl)-1-methyl-1H-imidazole-5-carboxamide—Compound 3.4 (85 mg, 0.16 mmol, 98%) as a white solid. ¹H NMR (400 MHz, DMSO) δ 10.56 (s, 1H), 8.01 (dd, J=6.8, 2.5 Hz, 1H), 7.70 (s, 1H), 7.62-7.55 (m, 1H), 7.42 (t, J=9.1 Hz, 1H), 6.01 (d, J=2.2 Hz, 1H), 5.56 (s, 1H), 5.26 (s, 1H), 4.52 (s, 1H), 3.73 (s, 2H), 3.69 (s, 3H), 3.65 (s, 3H), 3.19 (d, J=8.2 Hz, 1H), 2.85 (dd, J=16.1, 9.2 Hz, 1H), 2.62 (t, J=8.5 Hz, 1H), 2.44 (br s, 1H), 2.37-2.24 (m, 2H), 1.72 (td, J=13.0, 7.5 Hz, 2H), 1.13 (s, 6H).

All publications, patents and patent applications cited in this specification are incorporated herein by reference for the teaching to which such citation is used.

Test compounds for the experiments described herein were employed in free or salt form.

The specific responses observed may vary according to and depending on the particular active compound selected or whether there are present carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1. A method of synthesizing Compound I, or a salt thereof, comprising a step converting Compound 3.4 to Compound I, or a salt thereof:

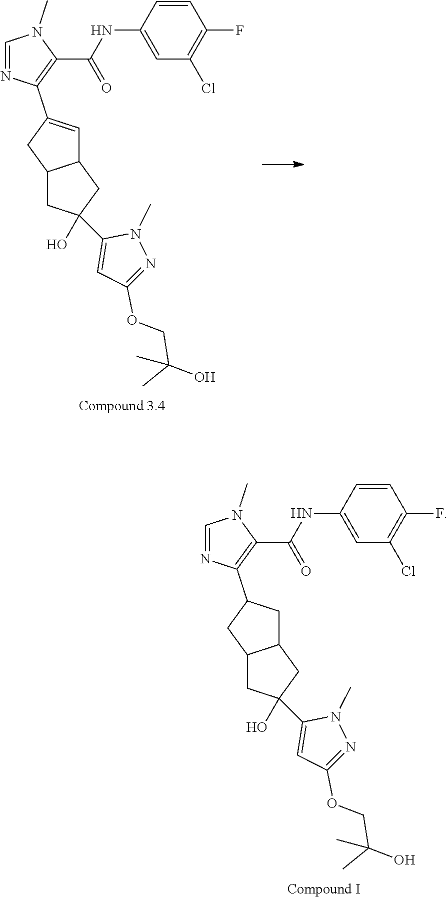 [SMILES: Cn1cnc(C2=CC3CC(O)(c4cc(OCC(C)(C)O)nn4C)CC3C2)c1C(=O)Nc1ccc(F)c(Cl)c1 ; Cn1cnc(C2CC3CC(O)(c4cc(OCC(C)(C)O)nn4C)CC3C2)c1C(=O)Nc1ccc(F)c(Cl)c1]

2. The method of claim 1, further comprising a step converting Compound 3.3 to Compound 3.4:

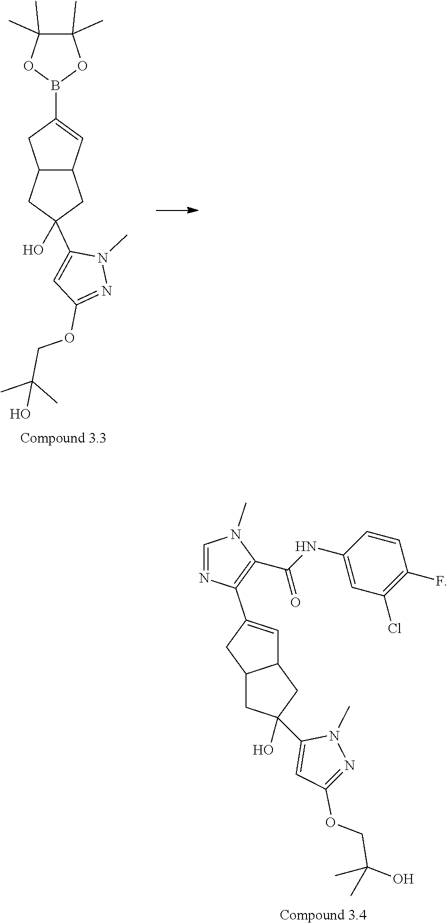 [SMILES: Cn1nc(OCC(C)(C)O)cc1C1(O)CC2C=C(B3OC(C)(C)C(C)(C)O3)CC2C1 ; Cn1cnc(C2=CC3CC(O)(c4cc(OCC(C)(C)O)nn4C)CC3C2)c1C(=O)Nc1ccc(F)c(Cl)c1]

3. The method of claim 2, further comprising a step converting Compound 3.2 to Compound 3.3:

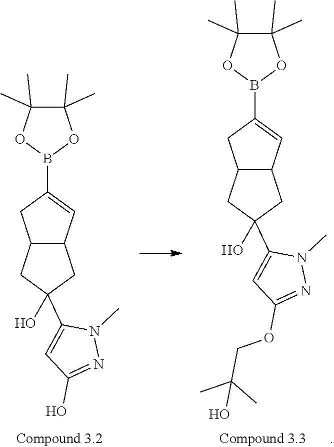

4. The method of claim 3, further comprising a step converting Compound 3.1 to Compound 3.2:

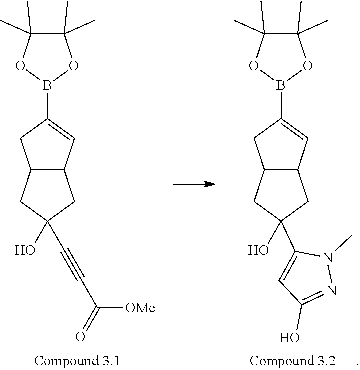

5. The method of claim 4, further comprising a step converting Compound 3 to Compound 3.1:

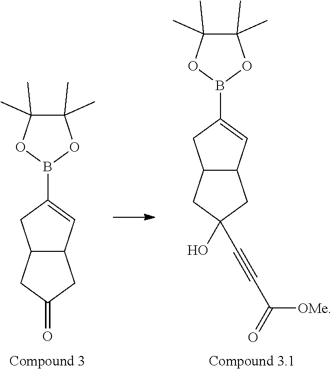

6. The method of claim 5, further comprising a step converting Compound 2 to Compound 3:

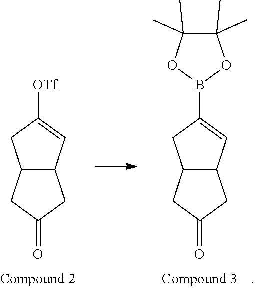

7. The method of claim 6, further comprising a step converting Compound 1 to Compound 2:

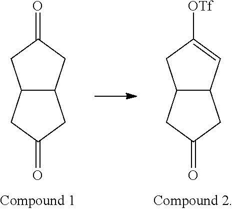 [SMILES: O=C1CC2CC(=O)CC2C1 ; O=C1CC2C=C(OS(=O)(=O)C(F)(F)F)CC2C1]

8. A method of synthesizing Compound Ia, or a salt thereof, comprising a step of converting Compound 3.4a to Compound Ia, or a salt thereof:

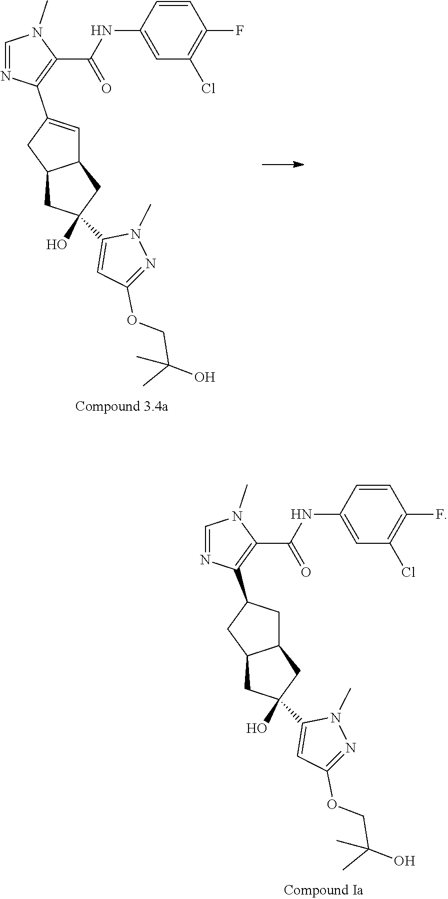 [SMILES: Cn1cnc(C2=C[C@H]3C[C@](O)(c4cc(OCC(C)(C)O)nn4C)C[C@H]3C2)c1C(=O)Nc1ccc(F)c(Cl)c1 ; Cn1cnc([C@@H]2C[C@@H]3C[C@@](O)(c4cc(OCC(C)(C)O)nn4C)C[C@@H]3C2)c1C(=O)Nc1ccc(F)c(Cl)c1]

9. The method of claim 8, further comprising a step converting Compound 3.3a to Compound 3.4a:

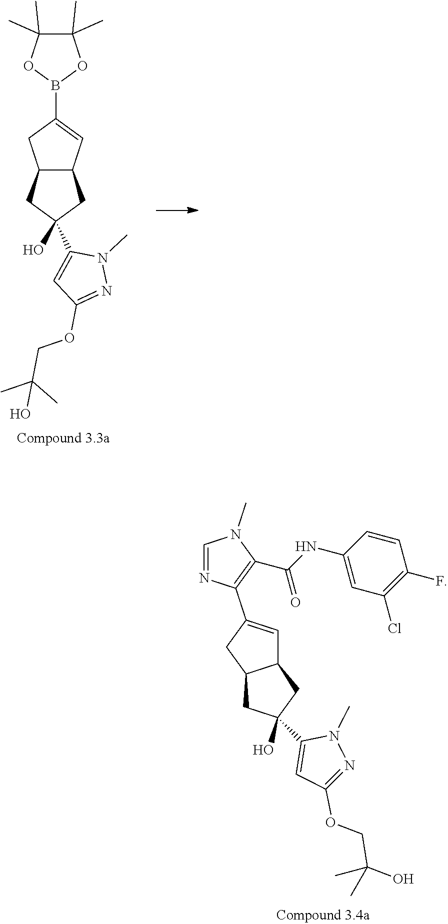 [SMILES: Cn1nc(OCC(C)(C)O)cc1[C@]1(O)C[C@H]2CC(B3OC(C)(C)C(C)(C)O3)=C[C@H]2C1 ; Cn1cnc(C2=C[C@H]3C[C@](O)(c4cc(OCC(C)(C)O)nn4C)C[C@H]3C2)c1C(=O)Nc1ccc(F)c(Cl)c1]

10. The method of claim 9, further comprising a step converting Compound 3.2a to Compound 3.3a:

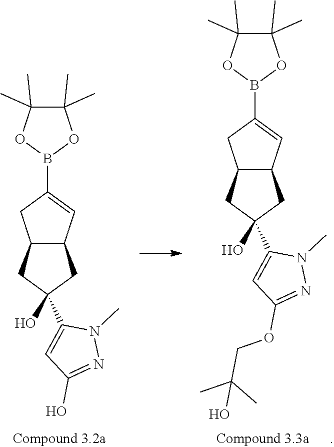

11. The method of claim 10, further comprising a step converting Compound 3.1a to Compound 3.2a:

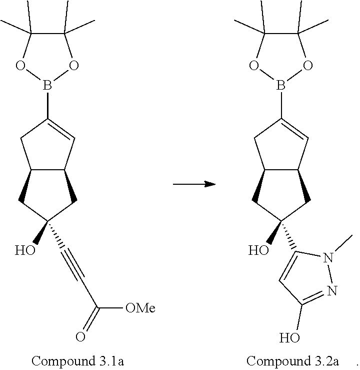

12. The method of claim 11, further comprising a step converting Compound 3a to Compound 3.1a:

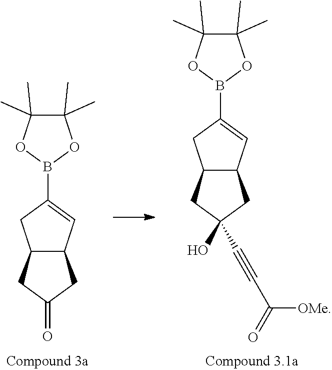

13. The method of claim 12, further comprising a step converting Compound 2a to Compound 3a:

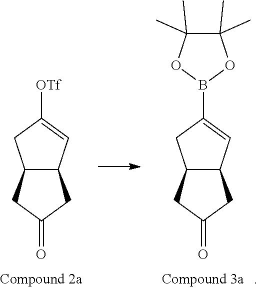

14. The method of claim 13, further comprising a step converting Compound 1a to Compound 2a:

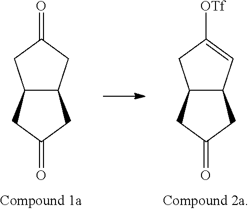 [SMILES: O=C1C[C@H]2CC(=O)C[C@H]2C1 ; O=C1C[C@H]2CC(OS(=O)(=O)C(F)(F)F)=C[C@H]2C1]

15. Compound I, or a salt thereof, obtained by, or obtainable by, a method according to claim 1.

16. Compound I, or a salt thereof, according to claim 15, wherein Compound I is substantially Compound I(a).

17. Compound I, or a salt thereof, according to claim 16, wherein Compound I comprises Compound I(a) and less than 10% by HPLC area of the other stereoisomers.

18. Compound Ia, or a salt thereof, obtained by, or obtainable by, a method according to claim 8.

19. Compound Ia, or a salt thereof, according to claim 18, wherein Compound Ia is stereochemically pure.

20. Compound 3.4, Compound 3.4a, or a salt thereof:

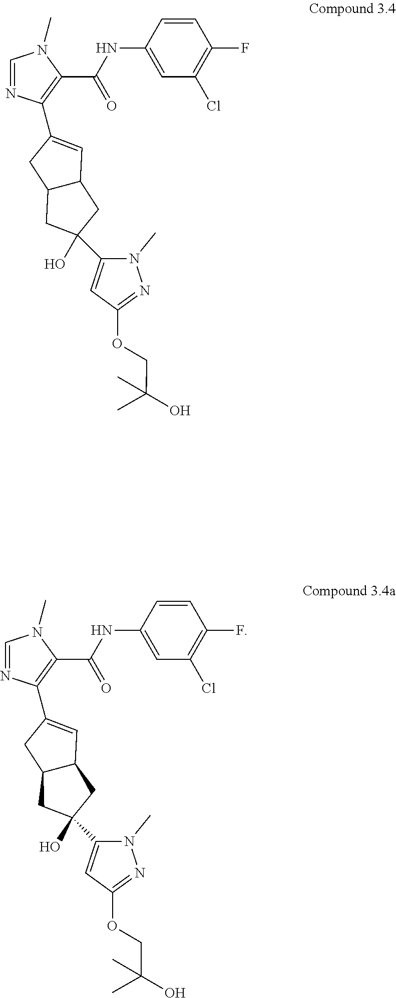 [SMILES: Cn1cnc(C2=CC3CC(O)(c4cc(OCC(C)(C)O)nn4C)CC3C2)c1C(=O)Nc1ccc(F)c(Cl)c1 ; Cn1cnc(C2=C[C@H]3C[C@](O)(c4cc(OCC(C)(C)O)nn4C)C[C@H]3C2)c1C(=O)Nc1ccc(F)c(Cl)c1]

21. (canceled)

22. A compound selected from one of the following compounds, or a salt thereof: 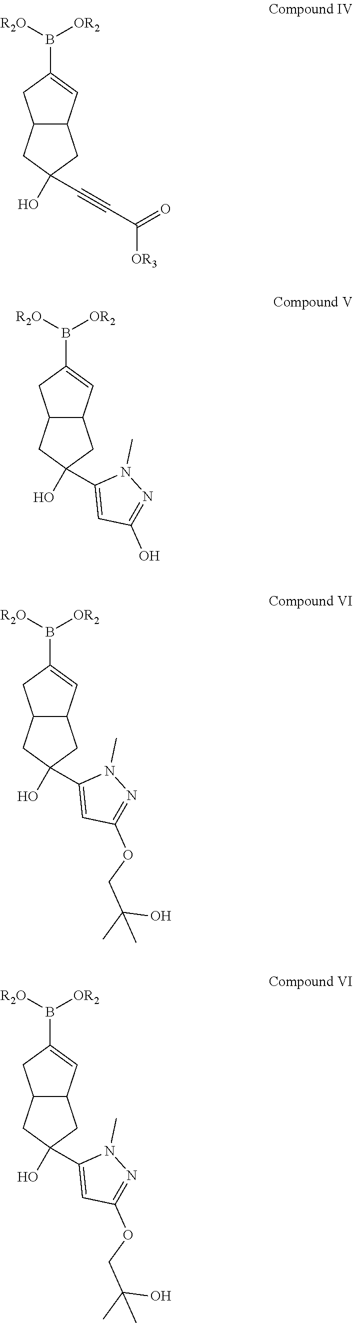 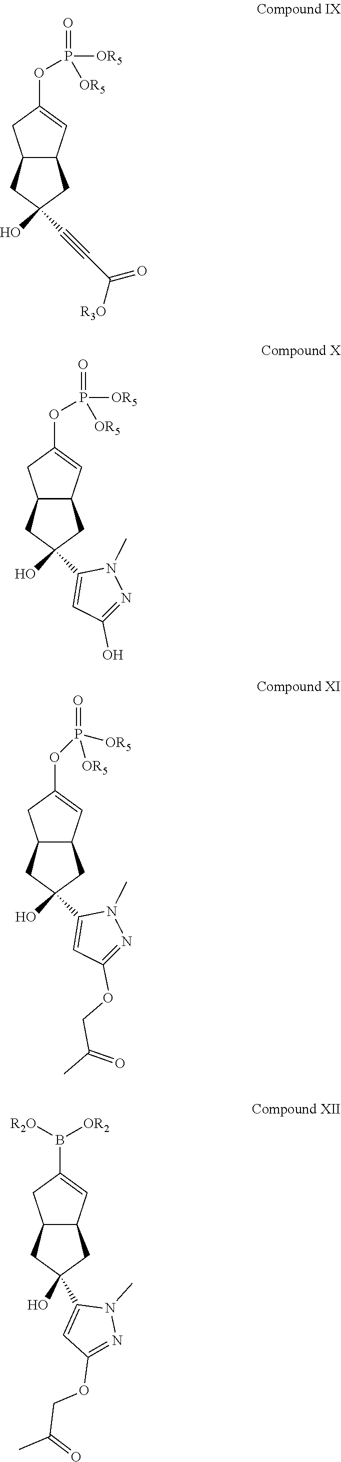 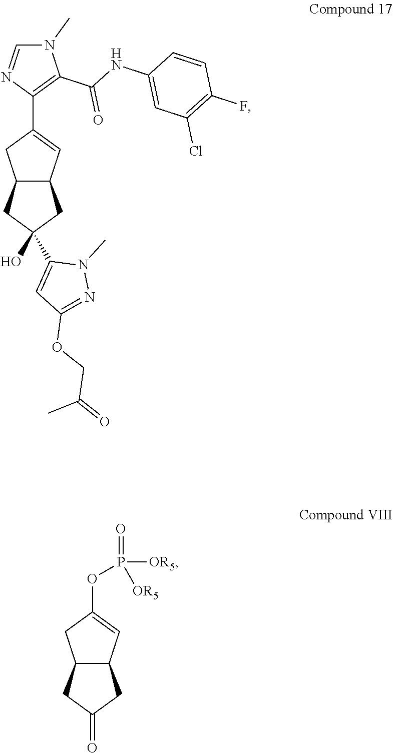
wherein each R₂ is independently hydrogen, alkyl or phenyl, or both R₂ are joined to form a cyclic boronic ester; R₃ is C₁₋₄alkyl; and R₅ is C₁₋₄alkyl or phenyl.

23. A compound according to claim 22, wherein both R₂ are joined to form a pinacol boronic ester;
R₃ is methyl or ethyl; and/or
R₅ is phenyl.

24. (canceled)

25. (canceled)

26. (canceled)
